(19) United States
(12) Patent Application Publication
(10) Pub. No.: US 2022/0226338 A1
(43) Pub. Date: Jul. 21, 2022

(54) Methods of Treating Cancer Using CHK1 Inhibitors

(71) Applicant: Sierra Oncology, Inc., San Mateo, CA (US)

(72) Inventors: Christian Andrew Hassig, San Mateo, CA (US); Bryan William Strouse, Plymouth, MI (US); Ryan James Hansen, San Mateo, CA (US); Kenna Lynn Anderes, San Mateo, CA (US); Snezana Milutinovic, San Mateo, CA (US); Angie J. You, San Mateo, CA (US); Barbara Jane Klencke, San Mateo, CA (US); Mark Kowalski, San Mateo, CA (US)

(73) Assignee: Sierra Oncology, Inc., San Mateo, CA (US)

(21) Appl. No.: 17/610,198

(22) PCT Filed: May 13, 2020
(86) PCT No.: PCT/US2020/032722

§ 371 Date: Nov. 10, 2021

Related U.S. Application Data
(60) Provisional application No. 62/855,910, filed on May 31, 2019.
(60) Provisional application No. 62/847,810, filed on May 14, 2019.

(51) Int. Cl. A61K 31/5377; A61K 31/7068; A61K 39/395; A61P 35/00; C12Q 1/6886

(52) U.S. Cl. CPC A61K 31/5377; A61K 31/7068; A61K 39/3955; C12Q 2600/156; C12Q 1/6886; C12Q 2600/106; A61P 35/00

(57) ABSTRACT

This disclosure provides methods of using a checkpoint kinase 1 (Chk1) inhibitor in the treatment of cancer in a subject having at least an intermediate tumor mutational burden (TMB), or a genetic abnormality in one or more particular genes associated with replicative stress. Accordingly, methods of treating cancer in a subject having at least an intermediate tumor mutational burden (TMB-I) are provided. Also provided are methods of treating cancer in a subject having a genetic abnormality in one or more particular genes selected from cell cycle regulation genes, replication stress genes, oncogenic driver mutations and DNA damage response and repair network genes. Methods of selecting subjects for Chk1 inhibition therapy are provided. The methods can include administering to the subject an effective amount of a SRA737 compound, in some cases in combination with low dose gemcitabine.

### CROSS-REFERENCE TO RELATED APPLICATIONS

This application claims priority to U.S. Provisional Application No. 62/847,810, filed on May 14, 2019; and U.S. Provisional Application No. 62/855,910, filed on May 31, 2019, both of which are hereby incorporated by reference in their entirety.

### INTRODUCTION

Cells activate a signal transduction pathway when DNA is damaged. Signals activate the cell-cycle machinery to induce DNA repair and/or cell death to mitigate propagation. Checkpoint kinase 1 (Chk1) is an important bridge in cells when sensing DNA damage. See Cancer Biology & Therapy (2004) 3:3, 305-313, incorporated herein by reference. Chk1 plays a role in regulating numerous and wide-ranging cellular functions including: immune and inflammation responses, spindle formation, DNA damage signal transduction and generally, cellular apoptosis. Chk1 inhibitors abrogate DNA damage-induced cell cycle arrest in S and/or G 2/M phases. Currently, there are no Chk1 inhibitors that are approved therapies for inhibition of tumor growth.

### SUMMARY

This disclosure provides methods of using a checkpoint kinase 1 (Chk1) inhibitor in the treatment of cancer in a subject having at least an intermediate tumor mutational burden (TMB), or a genetic abnormality in one or more particular genes associated with replicative stress. Accordingly, methods of treating cancer in a subject having at least an intermediate tumor mutational burden (TMB-I) are provided. Also provided are methods of treating cancer in a subject having a genetic abnormality in one or more particular genes selected from cell cycle regulation genes, replication stress genes, oncogenic driver mutations and DNA damage response and repair network genes. Methods of selecting subjects for Chk1 inhibition therapy are provided. The methods can include administering to the subject an effective amount of a SRA737 compound, in some cases in combination with low dose gemcitabine.

### BRIEF DESCRIPTION OF THE DRAWINGS

These and other features, aspects, and advantages of the present disclosure will become better understood with regard to the following description, and accompanying drawings.

FIG. 1 shows the effect of SRA737 on gemcitabine induced CHK1 S296 autophosphorylation in HT29 human tumor xenografts in mice as measured by western blotting.

FIG. 2 shows SRA737 dose proportional plasma concentrations and tumor concentrations in a HT29 xenograft model. For each dose, columns from left to right are concentrations for plasma at 6 hours, plasma at 24 hours, tumor at 6 hours, and tumor at 24 hours, respectively.

FIG. 3A-3B show details of the dosage used in study of SRA737 therapy in combination with low dose gemcitabine. FIG. 3A shows doses used for the cohorts, where MED is the minimum effective dose of SRA737 modeled from preclinical studies. FIG. 3B shows a graph of gemcitabine dose (mg/m²) which illustrates that relative to standard-of-care, gemcitabine doses tested in SRA737-02 were approximately 10-25% of a standard cytotoxic dose, e.g., a sub-therapeutic dose.

FIG. 4 shows immunohistochemistry (IHC) analysis of formalin fixed paraffin embedded (FFPE) tissue samples from HT-29 tumor bearing mice. IHC analysis shows phosphorylated Chk1 (p-Chk1) on Serine 296 (FIG. 4A), Serine 317 (FIG. 4B), and Serine 345 (FIG. 4C) and phosphorylated H2AX (γ-H2A.X) (FIG. 4D).

FIG. 5A-5B shows synergy of SRA737 with gemcitabine in mice bearing subcutaneous colorectal HT-29 tumors treated with SRA737 and 20 mg/kg gemcitabine (FIG. 5A), or SRA737 and 100 mg/kg gemcitabine (FIG. 5B).

FIG. 6 shows a summary of the baseline characteristics and demographics of subjects in the SRA737 monotherapy study (SRA737-01).

FIG. 7 illustrates the serum concentrations achieved using different doses of SRA737 monotherapy (SRA737-01).

FIG. 8 shows a plot indicating duration of treatment of SRA737 monotherapy (SRA737-01) for all subjects (N=107).

FIG. 9 shows directionally positive HGSOC cohort responses. The waterfall plot shows the best % tumor change from baseline among evaluable HGSOC subjects. Gene alterations across the FA/BRCA, PI3K and CCNE gene networks are noted below the graph for each individual subject. V is adjudicated VUS, as described in the experimental section.

FIG. 10 shows a summary of disease control rates (DCR) which vary across defined gene networks for SRA737 monotherapy (SRA737-01). This table summarizes the DCR across 11 gene networks within the three functional gene categories: Cell Cycle Dysregulation, Oncogenic Drivers and the DNA Damage Response and Repair Network. Individual genes of interest within gene networks are indicated. ‡ Rate for RAS wild-type subjects. * includes FANC A, C, and G. ** includes RAD51 and RAD51C.

FIG. 11 illustrates the frequency of gene network alterations across indications for SRA737 monotherapy (SRA737-01). This heatmap displays the frequency of observed gene network alterations across the treatment cohorts, represented by percent of subjects with gene alterations.

FIG. 12 illustrates that RAS gene network alterations may be antagonistic to SRA737 monotherapy sensitivity. The waterfall plot shows the best % tumor change from baseline among evaluable subjects having various cancers and harboring RAS gene network alterations. Genetic alterations in specific RAS genes and cancers for each subject are noted below the waterfall. CRC is colorectal cancer, HGSOC is high grade serous ovarian cancer, and NSCLC is non-small cell lung cancer. SD is stable disease and PD is progressive disease.

FIG. 13 shows a waterfall plot indicating PI3K gene network alterations may enhance SRA737 monotherapy sensitivity. Data shown represent the best % tumor change from baseline among evaluable subjects harboring PI3K/AKT subclass (e.g., AKT1, AKT2, AKT3, PIK3CA and/or PTEN variant) gene network alterations. Specific PI3K network gene alterations for each subject are noted below the waterfall. mCRPC is metastatic castration-resistant prostate cancer.

FIG. 14 illustrates FA/BRCA replication fork gene network alterations associated with SRA737 monotherapy activity. The waterfall plot shows the best % tumor change from baseline among evaluable subjects harboring FA/BRCA subclass gene network alterations. Specific FA/BRCA network gene alterations (e.g., AKT, PIK3CA and/or PTEN) for each subject are noted below the waterfall. mCRPC is metastatic castration-resistant prostate cancer.

FIG. 15 shows a summary of subjects with notable responses and their respective gene network alterations. Subjects who achieved SD of ≥4 cycles and/or best % tumor decrease of >10% are correlated with their respective gene network alterations.

FIG. 16 shows a summary of the baseline characteristics and demographics of subjects in the SRA737+LDG combination study (SRA737-02).

FIG. 17 shows data from human subjects demonstrating efficacy of SRA737 therapy in combination with low dose gemcitabine across multiple indications. Data shown represents the best % tumor change from baseline among evaluable subjects within the four tumor types (anogenital, cervical, rectal, high grade serous ovarian cancer (HCSOC). The magnitude of target tumor decrease in anogenital tumors was notable; two subjects with ongoing decreases of −66%* and −51% respectively, and a third subject with decrease of −41%* were observed as of the data cutoff. Data cut off: 3 May 2019. * confirmed Partial Response (PR).

FIG. 18 shows a summary of response and disease control rates (DCR) which vary across gene networks surveyed. This table summarizes the DCR and response rates across 11 gene networks within the three functional gene categories: Cell Cycle Dysregulation, Oncogenic Drivers and the DNA Damage Response and Repair Network. Individual genes within gene networks are indicated. ‡ Rate for RAS wild-type subjects. * includes FANC A, C, E, I and M. ** includes RAD51.

FIG. 19 illustrates PI3K gene network alterations may enhance sensitivity to SRA737+LDG therapy. The waterfall plot shows the best % tumor change from baseline among evaluable subjects harboring PI3K gene network alterations. Individual genes altered in each subject's tumor are noted below the waterfall.

FIG. 20 illustrates the FA/BRCA replication fork gene network associated with SRA737+LDG activity. The waterfall plot shows the best % tumor change from baseline in sum of target tumor diameters among evaluable subjects harboring FA/BRCA gene network alterations. Individual genes altered in each subject's tumor are noted below the waterfall. V refers to adjudicated VUS.

FIG. 21 is a schematic showing tumor reductions and clinical responses to SRA737+LDG correlate with mutations in FA/BRCA gene network. FA/BRCA gene network encodes for proteins that respond to RS by stabilizing and repairing stalled replication forks and HRR in conjunction with Chk1 and other DDR checkpoint kinases. Genetic alterations in these complexes contribute directly to RS, increasing genomic instability that manifests as increased tumor mutational burden (TMB) in certain contexts.

FIG. 22 shows a summary of the frequency of gene network alterations assessed for individual subjects undergoing SRA737+LDG combination therapy (SRA737-02) across indications of interest. This heatmap displays the frequency of observed gene network alterations across the treatment cohorts, represented by percent of subjects with gene alterations.

FIG. 23 illustrates SRA737+LDG therapy provides responses in squamous anogenital and cervical cancer. The waterfall plot shows the best % tumor change from baseline in sum of target tumor diameter among evaluable subjects with squamous anogenital and squamous cervical cancer. Data shown represent Gene network alterations in FA/BRCA or PI3K, HPV status and TMB status are noted in heatmap below for each subject. Subjects of interest can have HPV positive cancer and/or cancer with at least an intermediate TMB (TMB-I/H).

FIG. 24 shows responses of subjects having anogenital cancer to SRA737+LDG therapy. The waterfall plot shows the best % tumor change from baseline among evaluable subjects with anogenital cancer from FIG. 23.

FIG. 25, panels A-B shows images of a 70 year old male with anal cancer and extensive liver metastasis. Prior therapy: radiation and 1 line of systemic therapy. Genetic Profile: FA/BRCA, PI3K and TMB-I. Panel A shows baseline image. Panel B shows image after SRA737+LDG therapy. Best tumor response: −41%. Duration on treatment: 11 cycles (response ongoing at discontinuation; patient decision).

FIG. 26, panels A-B, show images of a 59 year old female with anal cancer and mediastinal mass compression & pleural effusion. Panel A shows baseline image. Panel B shows image after cycle 2 of SRA737+LDG therapy. Prior therapy: 3 lines of systemic therapy. Genetic Profile: FA/BRCA and TMB-I. Best tumor response: −26%+resolution of pleural effusion. Duration on treatment: 7 cycles; ongoing (as of data cut off: 3 May 2019).

FIG. 27A-27B show data from individual human subjects treated with SRA737 monotherapy (SRA737-01) (FIG. 27B, NSCLC subjects), or SRA737 in combination with low dose gemcitabine (LDG) (SRA737-02) (FIG. 27A and FIG. 27B, Anal and rectal cancer subjects). The data indicates subjects having various cancers and an intermediate TMB (TMB-I) or higher (TMB-H) can be responsive to treatment with SRA737 in combination with LDG.

### DETAILED DESCRIPTION

This disclosure provides methods of treating cancer using a checkpoint kinase 1 (Chk1) inhibitor in a subject having at least an intermediate tumor mutational burden (TMB), or a genetic abnormality in one or more particular genes associated with replicative stress. The genetic abnormalities may be found in one or more genes selected from the classes of cell cycle regulation genes, replication stress genes, oncogenic driver mutations and/or DNA damage response and repair network genes. Methods of selecting subjects for Chk1 inhibition therapy are also provided. The methods can include administering to the subject an effective amount of a SRA737 compound, in some cases, in combination with low dose gemcitabine.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that, as used in the specification and the appended claims, the singular forms “a,” “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods of Treating Cancer

Aspects of this disclosure include methods of treating cancer using a Chk1 inhibitor in a subject identified as having at least an intermediate TMB, or a genetic abnormality in one or more particular genes associated with replicative stress, which biomarkers can be indicative of sensitivity to Chk1 inhibition therapy. The subject methods can include administration of a Chk1 inhibitor, such as SRA737, optionally in a combination therapy with low dose gemcitabine (LDG). Also provided are methods of selecting a subject who would benefit from Chk1 inhibition (Chk1i) therapy.

The present disclosure provides results from first-in-human clinical studies of Chk1 inhibitor therapies: SRA737 monotherapy (study SRA737-01), and SRA737 combination therapy with low dose gemcitabine (study SRA737-02; SRA737+LDG). Multiple solid tumor indications were evaluated on the basis of Chk1i sensitivity and/or prevalence of replicative stress (RS)-associated tumor genomics. Subjects having tumors harboring RS-associated genetic alterations and/or other backgrounds implicated in Chk1i sensitivity were evaluated. The clinical study response data and tumor genomics of individual patients were analyzed to identify indication-specific genomic signatures indicative of enriched response to a SRA737 therapy. Accordingly, the present disclosure provides methods of treating cancer in subjects having a particular genetic abnormality with Chk1 inhibition therapy, such as SRA737 in combination with low dose gemcitabine.

### Replication Stress Driver Genes

This disclosure provides genetic abnormalities in replicative stress (RS) driver genes which can be indicative of responsiveness to Chk1 inhibition therapy. Aspects of the present disclosure include methods of treating cancer with a Chk1 inhibitor in a subject identified as having one or more such genetic abnormalities in a gene indicative of sensitivity to Chk1 inhibition (e.g., as described herein).

Chk1 is a master regulator of replication stress (RS). Chk1 is a serine/threonine protein kinase in the DNA Damage Response (DDR) network that can reduce elevated replication stress in certain tumor cells. Replication stress (RS) is manifested by the slowing and stalling of replication forks which results in fragile, exposed single-stranded DNA that is prone to damage. Increased RS results in genomic instability, which affords certain growth and survival advantages to tumor cells, however, if not properly managed, can result in extensive DNA damage and cell death. Consequently, tumor cells increase reliance on Chk1 to manage elevated intrinsic RS. Cancer cells with higher RS may have increased sensitivity to Chk1 inhibitor therapy.

Drivers of RS which can find use in the selection of subjects for treatment according to the subject methods include genetic abnormalities in tumor suppressors, oncogenic drivers, and/or DNA damage response and repair network genes. Tumors harboring defects in these functional gene networks can have higher levels of intrinsic RS due to dysregulated cell cycle control, increased proliferation demands and/or increased genomic instability. These RS driver genes can be divided into several functional categories including G1/S tumor suppressors, oncogenic drivers and defective DNA damage response and repair genes.

The present disclosure provides RS driver genes that can lead to an increased reliance on Chk1 in tumor cells of interest. Subjects identified as having such tumor cells can be treated with Chk1 therapy according to the methods described herein. In some cases, the Chk1 therapy utilized in the subject methods is a combination therapy of a Chk1 inhibitor with an extrinsic RS inducer that depletes replication building blocks, such as low dose gemcitabine.

Accordingly, a patient selected for treatment according to the subject methods can have one or more genetic abnormalities in one or more of the RS inducer genes described herein.

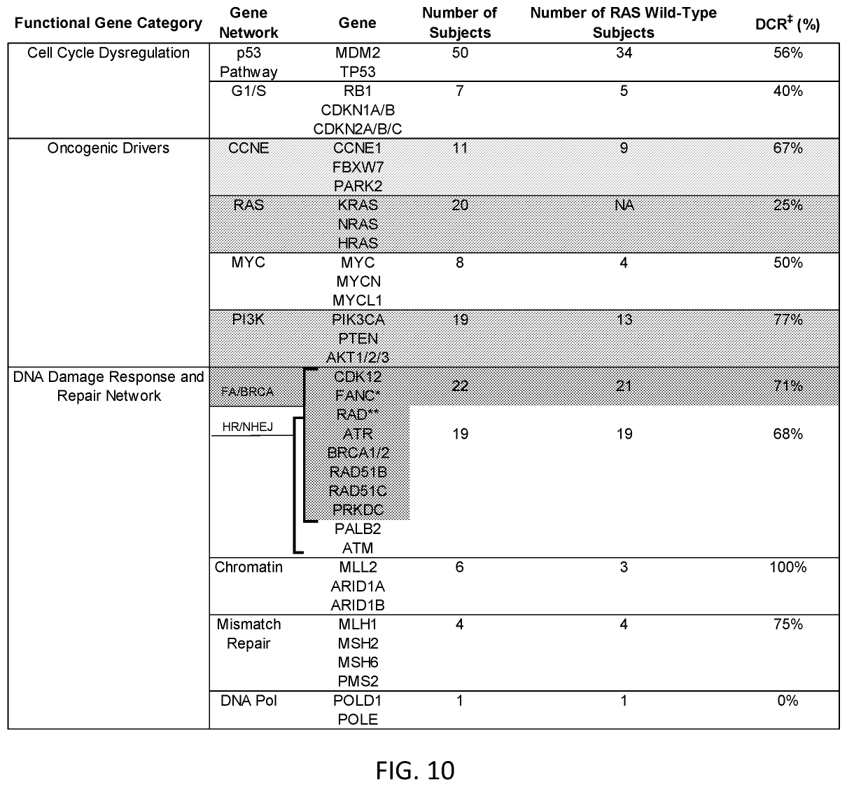

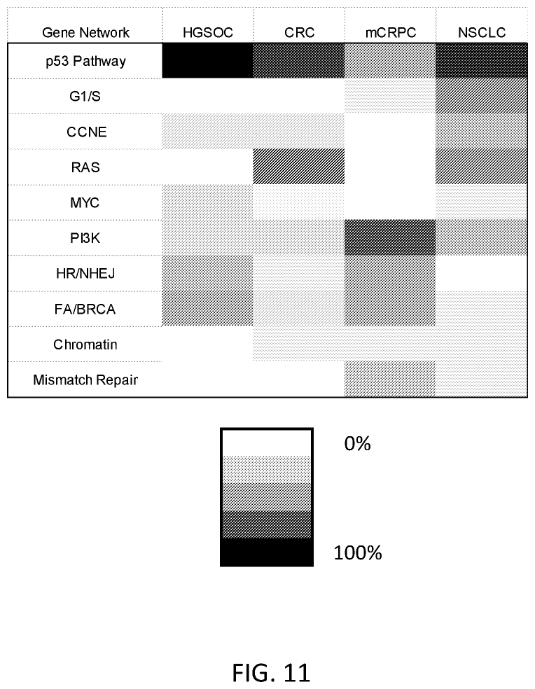

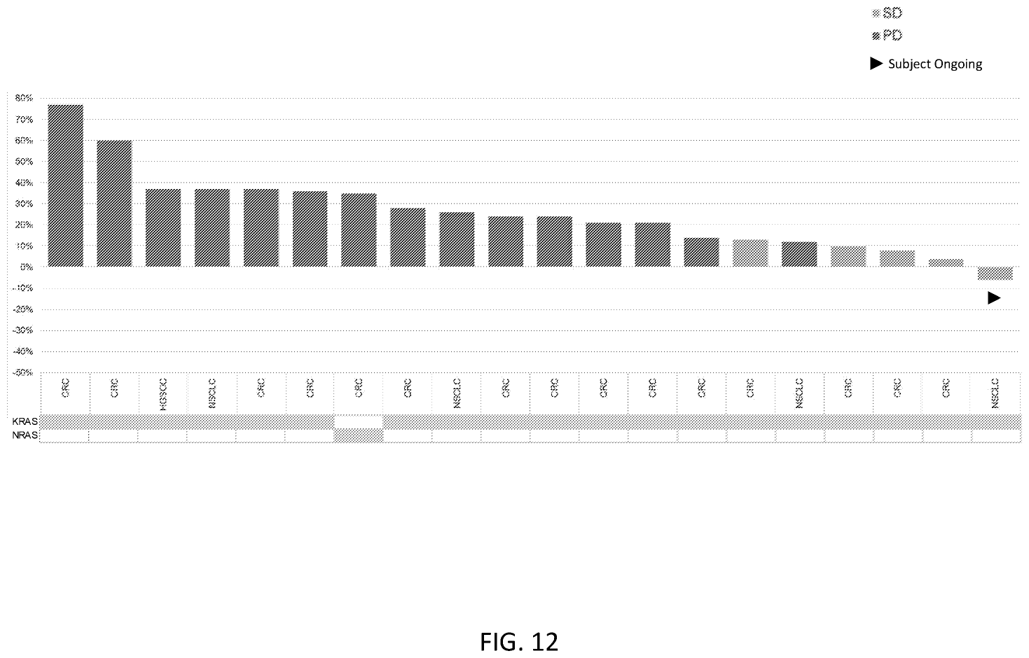

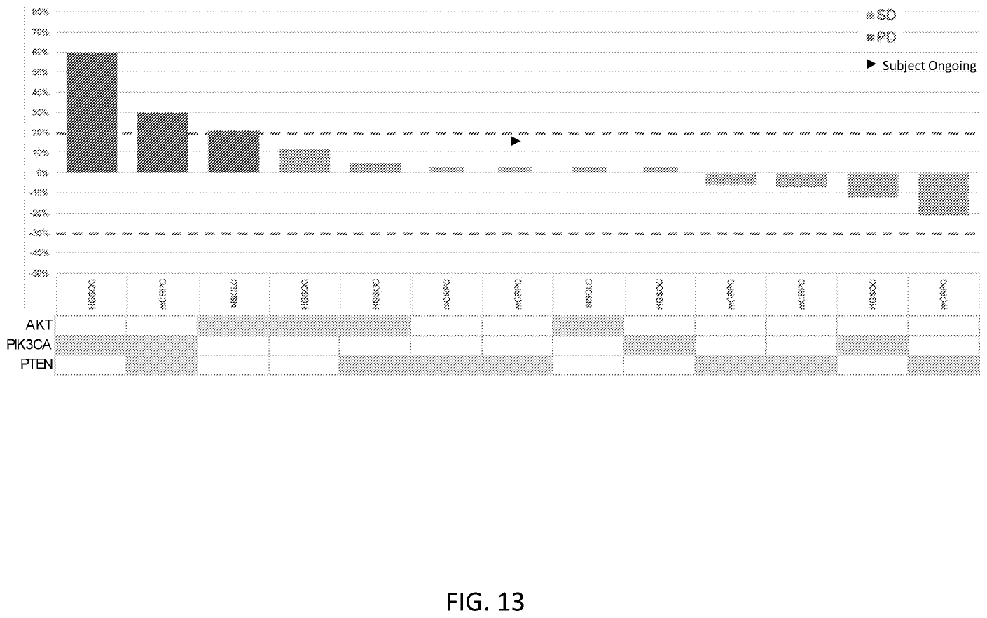

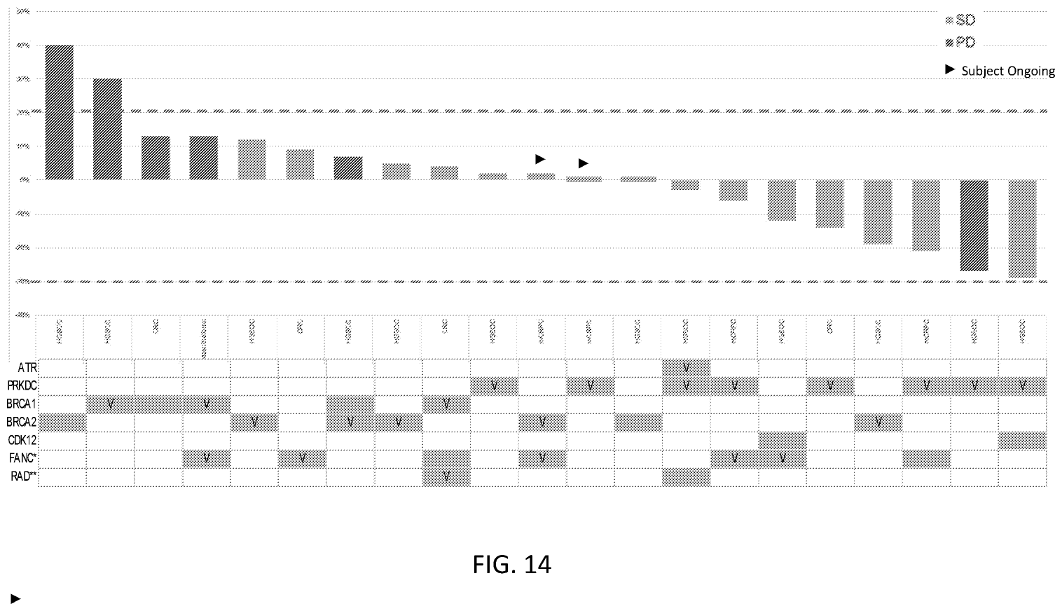

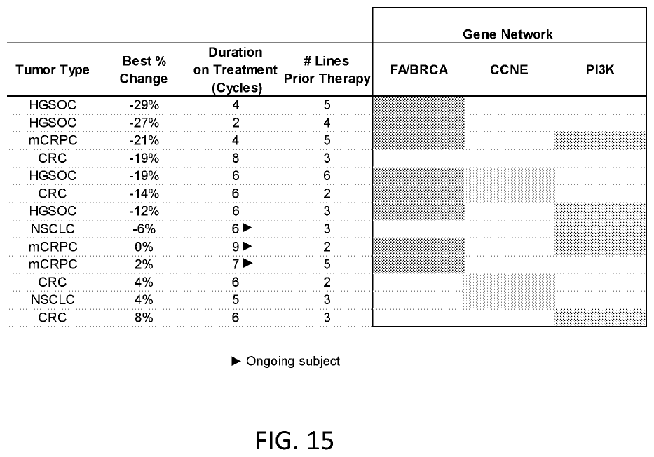

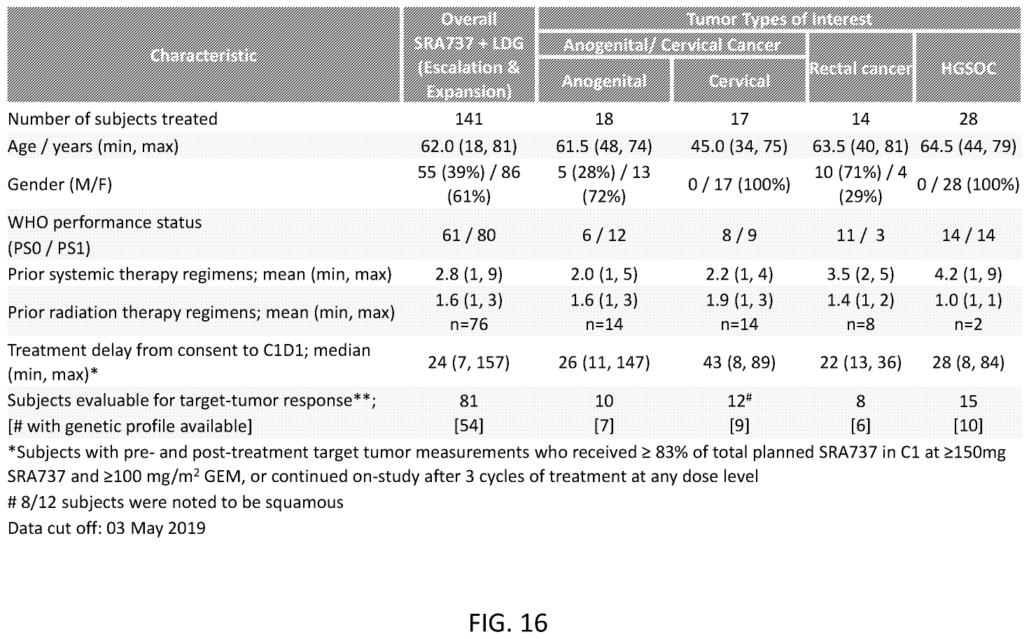

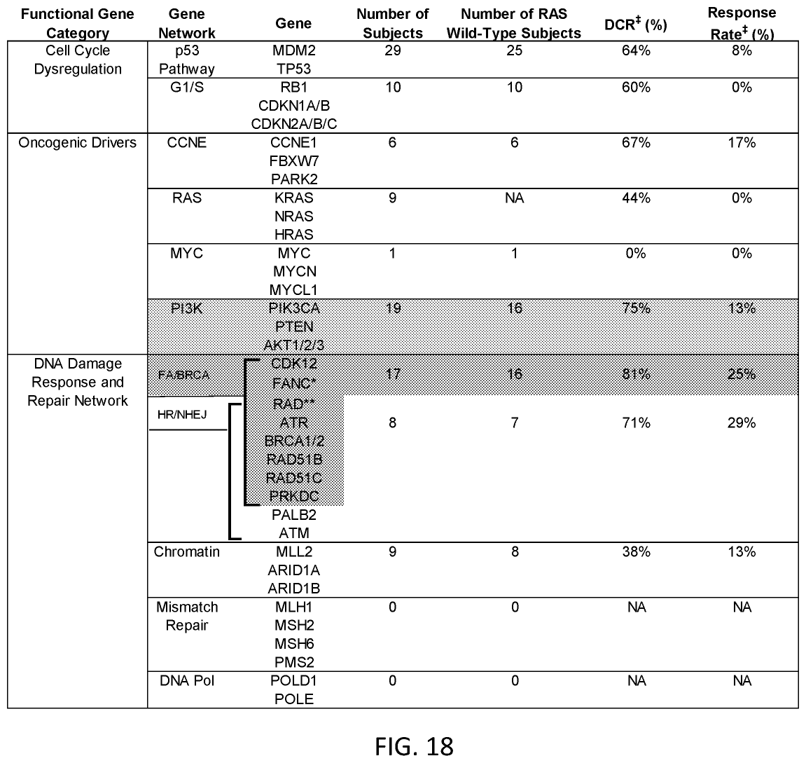

Classes of intrinsic RS inducer genes of interest include, but are not limited to, cell cycle dysregulation mutations (e.g., of the p53 pathway subclass or G1/S subclass), oncogenic driver mutations (e.g., of the CCNE, MYC, and/or PI3K/AKT subclasses) and DNA damage response and repair network mutations (e.g., of the HR/NHEJ, FANC/BRCA replication fork, chromatin, and/or mismatch repair subclasses). See e.g., FIG. 10 and FIG. 18.

RS inducer genes include the class of cell cycle dysregulation. Cell cycle dysregulation genes of interest include, but are not limited to, the p53 pathway subclass, and the G1/S subclass.

The p53 tumor suppressor protein can function as a transcription factor to transactivate or repress a variety of target genes. The downstream targets of p53 can regulate the pathways of cell cycle arrest, apoptosis, and DNA repair to maintain a dynamic equilibrium between cell growth and arrest in response to factors including DNA damage, hypoxia (oxygen deprivation), and a deficiency of growth factors or nutrient. Particular genes of interest of the p53 cell cycle dysregulation subclass in which mutations can increase tumor cell reliance on Chk1 include, but are not limited to, MDM2 (MDM2 proto-oncogene) and TP53 (tumor protein p53).

The G1/S transition is a stage in the cell cycle between the G1 growth phase and the S phase of DNA replication. It is governed by cell cycle checkpoints to ensure cell cycle integrity and the subsequent S phase can pause in response to improperly or partially replicated DNA. Particular genes of interest of the G1/S cell cycle dysregulation subclass in which mutations can increase tumor cell reliance on Chk1 include, but are not limited to, RB1 (RB transcriptional corepressor 1), CDKN1A/B (cyclin dependent kinase inhibitor 1A/1B) and CDKN2A/B/C (cyclin dependent kinase inhibitor 2A/2B/2C).

RS inducer genes include the class of oncogenic drivers. Oncogenic driver genes of interest include, but are not limited to, CCNE (cyclin E), MYC, and/or PI3K/AKT subclasses. Cyclin E forms a complex with cyclin-dependent kinase (CDK2). Cyclin E/CDK2 regulates multiple cellular processes by phosphorylating numerous downstream proteins, and plays a role in the G1 phase and in the G1-S phase transition. Over-expression of cyclin E (CCNE) can correlate with tumorigenesis. Myc (or MYC) is a family of regulator genes and proto-oncogenes that code for transcription factors and includes three related human genes: c-myc, 1-myc, and n-myc.

Protein kinase B, also known as PKB or AKT is a serine/threonine-specific protein kinase that plays a key role in multiple cellular processes such as glucose metabolism, apoptosis, cell proliferation, transcription and cell migration. The AKT signaling cascade is activated by receptor tyrosine kinases, integrins, B and T cell receptors, cytokine receptors, G-protein-coupled receptors and other stimuli that induce production of phosphatidylinositol (3,4,5) trisphosphates (PIP3) by phosphoinositide 3-kinase (PI3K). Phosphoinositide 3-kinases (PI3K) are a family of related intracellular signal transducer enzymes. Dysregulation of the PI3K/AKT pathway is implicated in a number of human diseases including cancer.

Intrinsic RS inducer genes include the class of DNA damage response and repair network mutations. DNA damage response and repair network genes of interest include, but are not limited to, the HIR/NHEJ, FA/BRCA (Fanconi anemia/Breast cancer susceptibility protein) replication fork, chromatin, and mismatch repair subclasses. It is understood that, in some cases, particular genes of interest may be considered part of two subclasses (e.g., the HR/NHEJ and FA/BRCA subclass as shown in FIG. 18) where there can be crossover of two gene networks.

The two major pathways for repair of DNA double-strand breaks are homologous recombination (HR) and nonhomologous end joining (NHEJ). Several genes are present in higher eukaryotes to regulate both pathways. HR/NHEJ subclass mutations of interest include, but are not limited to, PALB2, ATM, BRCA1/2, RAD51B, RAD51C and PRKDC, and ATR.

FA/BRCA (Fanconi anemia/Breast cancer susceptibility protein) replication fork subclass of mutations of interest include, but are not limited to, PRKDC, ATR, BRCA1/2, CDK12, FANC genes include FANC A, D2, E, G, I, or M and RAD genes including RAD52, RAD50, RAD51B, RAD51C and RAD54L.

Chromatin subclass of genes of interest includes, but are not limited to, MLL2, ARID1A and ARID1B. Mismatch repair subclass of genes of interest includes, but are not limited to, MLH1, MSH2, MSH6 and PMS2. DNA polymerase (DNA pol) subclass of genes of interest includes, but are not limited to, POLD1 and POLE.

Any convenient genetic abnormality in any of the target genes disclosed herein can be observed and considered a desirable marker of sensitivity. The genetic abnormality of interest can be an alteration, amplification, overexpression, or underexpression of the target gene. A variety of genetic abnormalities in RS inducer genes can be targeted. In some cases, the genetic abnormality is a gene alteration, e.g., a mutation.

In some embodiments of the method, the one or more genes is selected from cell cycle regulation genes associated with the G1/S checkpoint and/or the p53 pathway. In certain instances, the one or more genes is selected from MDM2, TP53, RB1, CDKN1A/B and CDKN2A/B/C.

In some embodiments of the method, the one or more genes is selected from replication stress genes implicated in Chk1 pathway sensitivity. In certain instances, the one or more genes is selected from Chk1 and ATR.

In some embodiments of the method, the one or more genes is selected from DNA damage response and repair genes associated with homologous recombination (HR), nonhomologous end joining (NHEJ), Fanconi anemia (FA), or mismatch repair, and a gene encoding chromatin or DNA polymerase. In certain instances, the one or more genes is selected from PALB2, ATM, BRCA1/A2, RAD51B, RAD51C, PRKDC, CDK12, FANCA, FANCD2, FANCE, FANCG, FANCI, FANCM, RAD52, RAD50, RAD51C, RAD54L, MLL2, ARID1A, ARID1B, MLH1, MSH2, MSH6, PMS2, POLD1, and POLE.

In some embodiments of the method, the one or more genes are oncogenic driver genes of the following subclasses: CCNE, MYC and PI3K/AKT. In certain instances, the one or more genes are of the CCNE subclass and selected from CCNE1, FBXW7, and PARK2.

In some embodiments of the method, the one or more genes are of the PI3K/AKT subclass and selected from PIK3CA, PTEN, AKT1, AKT2, and AKT3.

In some embodiments of the method, the one or more genes are of the MYC subclass and selected from MYC, MYCN, and MYCL1.

In some embodiments of the method, the subject is identified as having cancer cells with a genetic abnormality in one or more genes (e.g., 1, 2 or more genes) selected from DNA damage response and repair genes of the FA/BRCA replication fork subclass. FIG. 10 reveals that subjects undergoing SRA737 monotherapy whose tumors harbored FA/BRCA network mutations displayed favorable outcomes (DCR=71%; DOS=3.8 cycles). In certain instances, the one or more genes includes ATR or PRKDC.

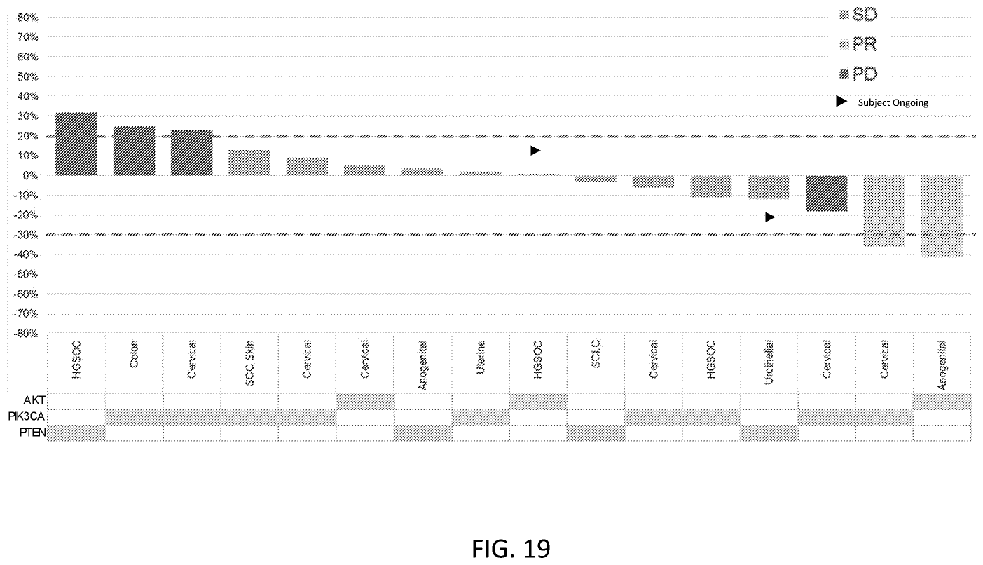

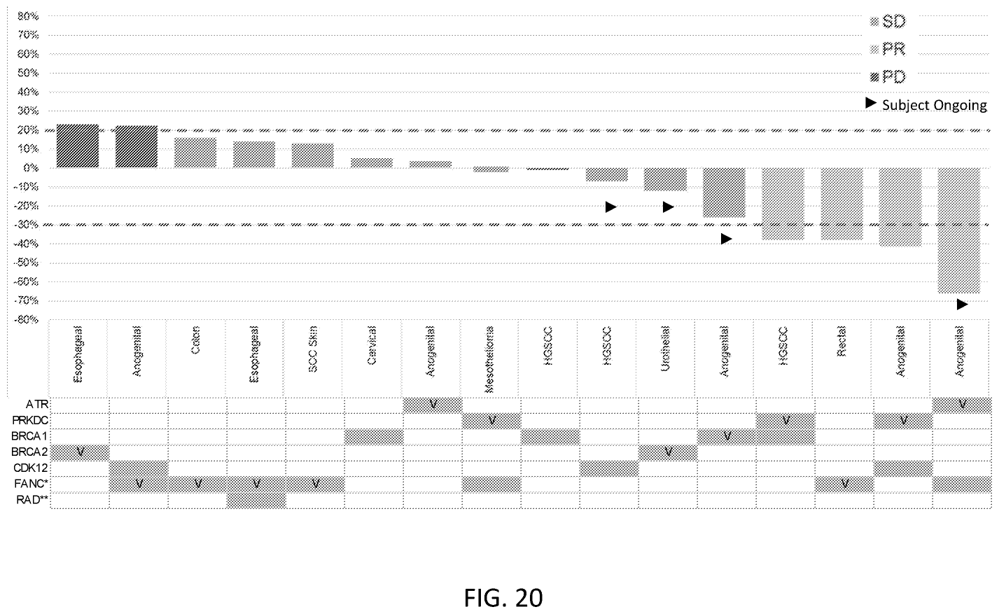

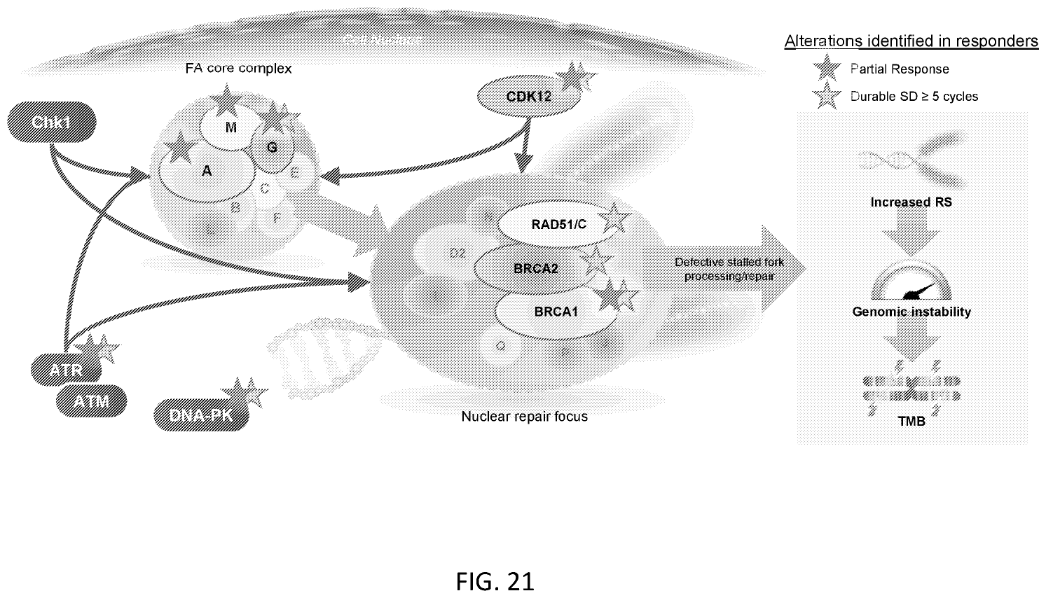

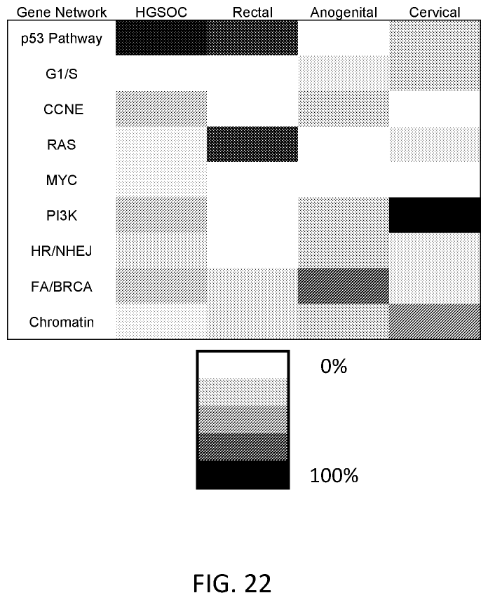

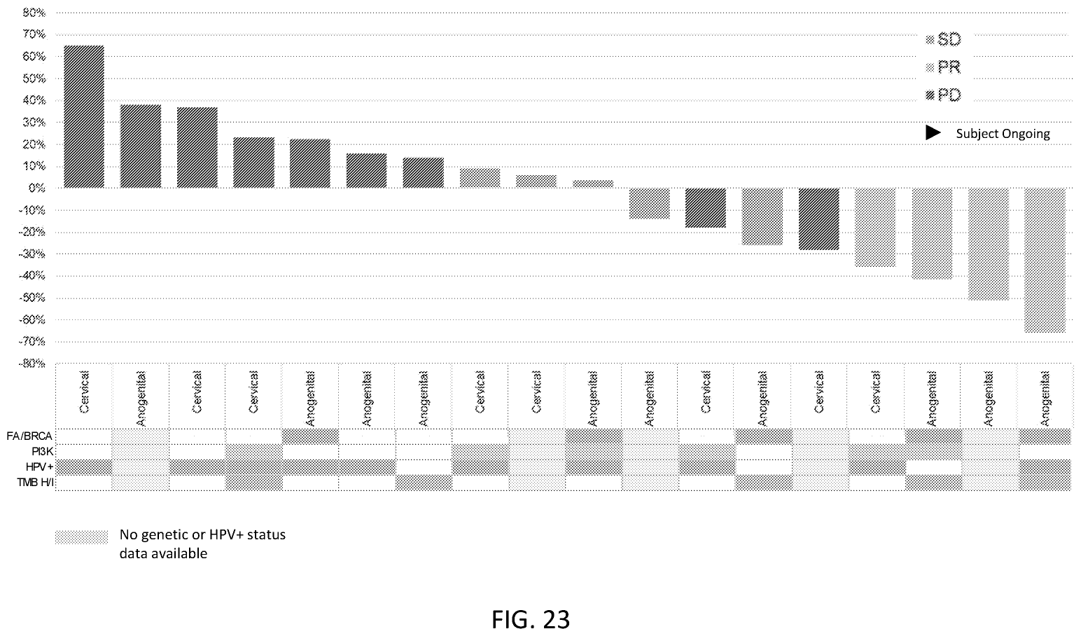

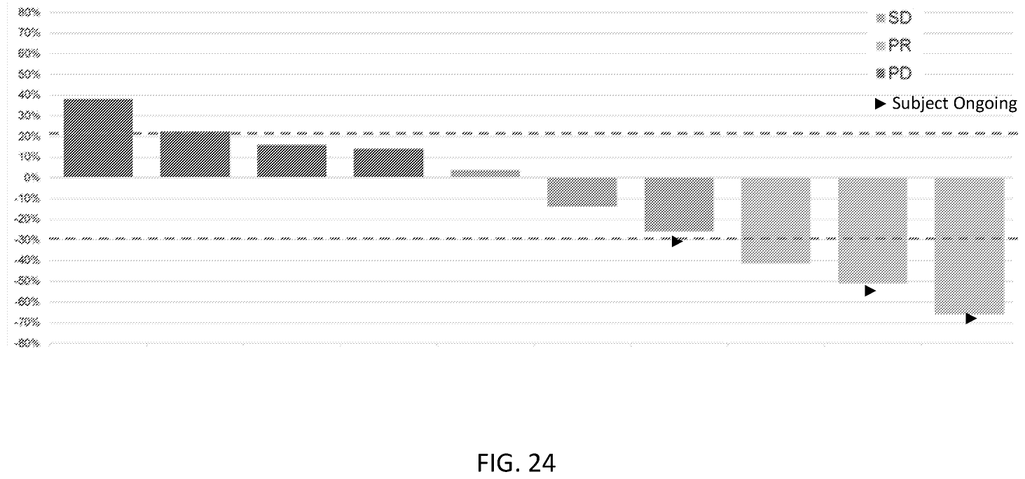

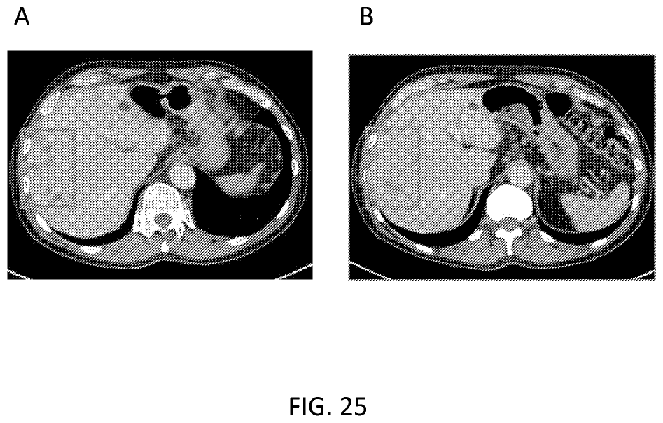

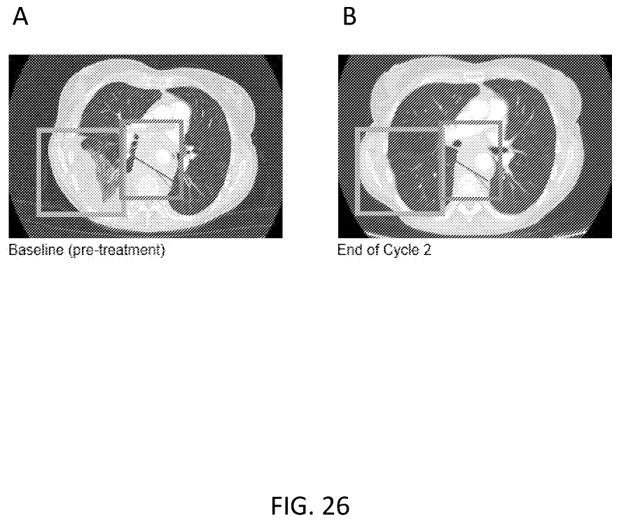

In some embodiments of the method, the subject is identified as having cancer cells with a genetic abnormality in two or more genes selected from oncogenic driver genes of the PI3K/AKT subclass and DNA damage response and repair genes of the FA/BRCA replication fork subclass. FIG. 18 shows a summary of response and disease control rates (DCR) which vary across gene networks surveyed. This table indicates the DCR and response rates to SRA737+LDG therapy for the subjects having a PI3K/AKT subclass genetic abnormality was 75% and 13%, respectively, while subjects having a FA/BRCA replication fork subclass gene abnormality was 81% and 25%, respectively. See also, FIG. 19-20 which show waterfall plots for SRA737+LDG therapy of various subjects having such genetic abnormalities. In certain embodiments of the method, the two or more genes are selected from AKT, PIK3CA, PTEN, ATR, PRKDC, BRCA1, BRCA2, CDK12, FANCA, FANCD2, FANCE, FANCG, FANCI, FANCM, RAD52, RAD50, RAD51C, and RAD54L.

In some embodiments of the method, the subject is further identified as having cancer positive for human papillomavirus (HPV). In some embodiments of the method, the subject is further identified as having at least an intermediate tumor mutational burden (TMB) (e.g., as described herein).

Aspects of the disclosure include determining the presence or absence of a genetic abnormality in a RAS gene (e.g., KRAS) in a sample obtained from the subject. Subjects having wild type RAS can be selected for treatment according to the methods of this disclosure. In some cases, a subject having cancer with a genetic abnormality (e.g., mutation) in a RAS gene is excluded from treatment. In some embodiments, the tumor cells of a subject treated according to the subject methods are identified as having wild type RAS. Accordingly, a subject selected for treatment can be a subject having tumor cells lacking any mutations in KRAS, NRAS and/or HRAS genes. In some cases, the KRAS mutation of interest is G12, G13, G34, G35, G37, G38, Q61, K117 or A146. In certain cases, the KRAS mutation of interest is G12C, G12R, G12S, G12A, G12D, G12V, G13C, G13R, G13S, G13A, G13D, G13V, G34T, G34A, G34C, G35T, G35C, G35A, G37T, G37C, G37A, G48T, G38A, G38A, Q61K, Q61L, Q61R, Q61H, K117N, A146P, A146T or A146V.

Genetic abnormalities can be assessed in a sample of the subject using any convenient methods. A variety of assays can be adapted for use in determining whether an alteration, amplification, overexpression, or underexpression of the one or more genes in present in the cancer of the subject. For example, the detection of substitutions, insertion-deletions (indels), and copy-number alterations (CNAs) of genes listed in Table 1 can achieved using the FoundationOne CDx assay. Methods of interest include those described in US2019/0085403, the disclosure of which is herein incorporated by reference.

In some instances, a sample is obtained from the subject for assessing or determining a genetic abnormality in the one or more genes of interest. The sample can be selected from tissue sample, whole blood sample, plasma sample, and serum sample. In some cases, the sample comprises tissue obtained from the subject. In certain instances, the sample comprises tumor cells. In some cases, the sample obtained from the subject contains at least 20% tumor cells.

### Intermediate Tumor Mutational Burden

A genetic abnormality biomarker that can be indicative of responsiveness to Chk1 inhibition therapy is tumor mutational burden (TMB). The TMB of a subject's cancer is based on a number of somatic mutations identified within the cancer genome. TMB values vary across a population of cancer subjects, but can be characterized according to the categories of low, intermediate, and high TMB levels. Based upon the results of the clinical studies described herein, it was determined that subjects having intermediate or higher levels of TMB have better clinical outcomes when treated the Chk1 inhibition therapy than subjects having low TMB levels.

Any convenient methods of assessing TMB can be utilized in conjunction with the subject methods. Methods of interest include those described in US2019/0085403, the disclosure of which is herein incorporated by reference. It is understood that the absolute value of TMB may vary depending on the method used to assess TMB in cancer cells of individual cancer subjects. In some cases, a whole cancer genome can be sequenced to identify a total number of somatic mutations. In certain cases, a subset of genes of interest within the cancer genome are targeted for assessment of somatic mutations. For example, described in the experimental section below is the FoundationOne CDx™ (F1CDx) assay, a next generation sequencing based in vitro diagnostic for detection of substitutions, insertion and deletion alterations (indels), and copy number alterations (CNAs) in up to 324 or more genes, as well as genomic signatures including tumor mutational burden (TMB) using DNA isolated from formalin-fixed paraffin embedded (FFPE) tumor tissue specimens.

Chalmers et al. (“Analysis of 100,000 human cancer genomes reveals the landscape of tumor mutational burden Genome Medicine, 2017, 9:34), the disclosure of which is herein incorporated by reference, compares TMB values determined in a targeted comprehensive genomic profiling assay (FoundationOne assay) to TMB values measured by whole exome sequencing methods (WES). Chalmers indicates that a CGP assay targeting ˜1.1 Mb of coding genome provides an accurate assessment TMB comparable with whole exome sequencing.

It is determined retrospectively that subjects having an intermediate level of TMB, as well as those subjects having a high level of TMB, can have better clinical outcomes when treated the Chk1 inhibition therapies described herein. Accordingly, in some embodiments, the subject treated according to the methods of this disclosure is one identified as having an intermediate level of tumor mutational burden (TMB-I) or higher, e.g., an intermediate or high TMB (TMB-I/H). As such, TMB-I can be a level of somatic mutation in a cancer cell of a subject indicative of responsiveness to Chk1 inhibitor therapy, e.g., as demonstrated herein.

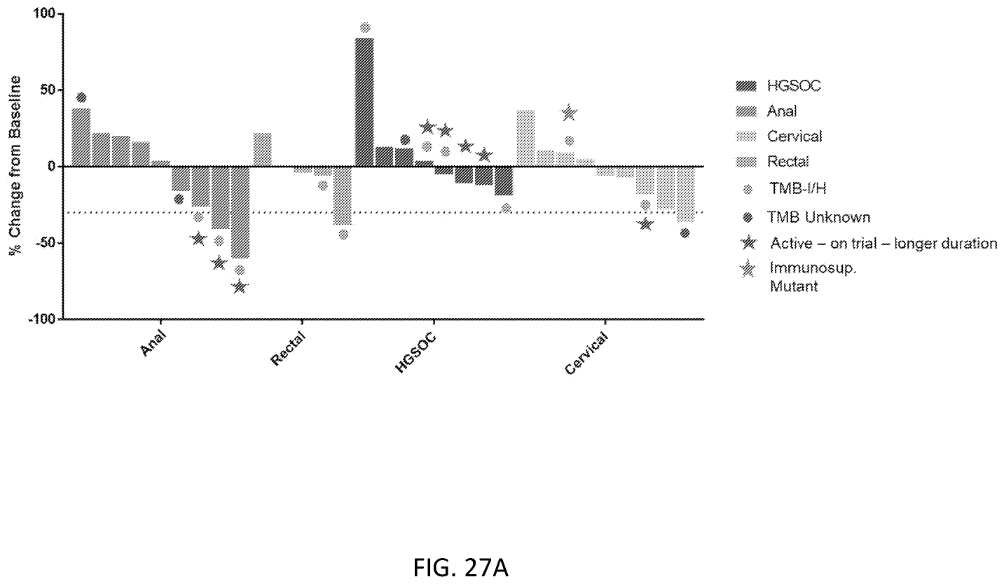

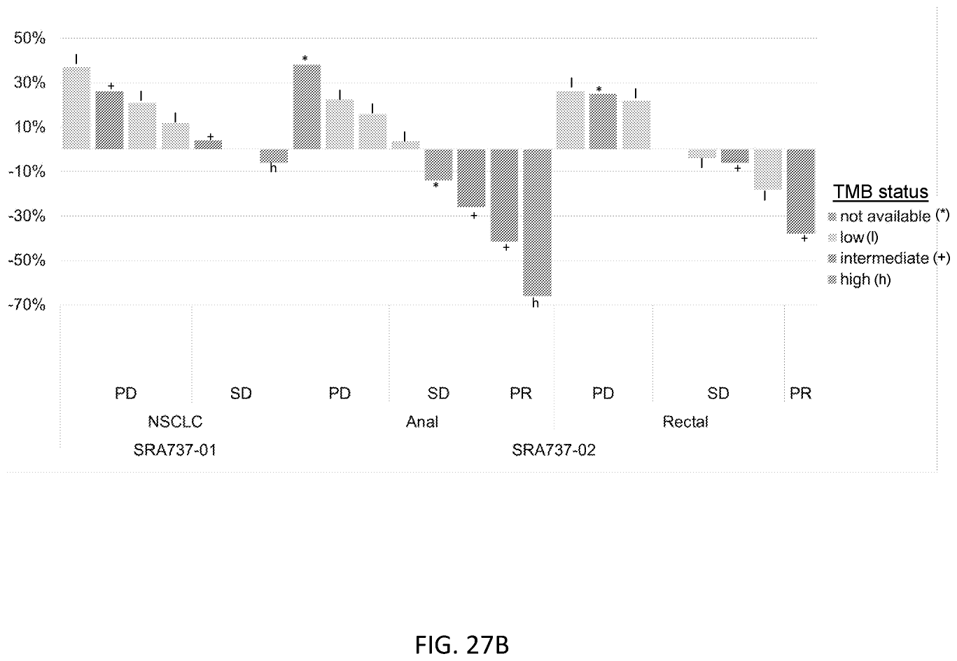

FIG. 27A-27B show waterfall plots of % change in tumor diameter from baseline during treatment of individual patients having various cancers with SRA737 in combination with low dose gemcitabine (LDG) (FIG. 27A and FIG. 27B, SRA737-02). In the plots, the TMB status of individual patients is indicated as determined using FoundationOne CDx™ (F1CDx) testing of TMB in patient samples, e.g., as described in the experimental section. For example, anal cancer subjects having intermediate or high TMB levels demonstrated enhanced responsiveness to treatment than subjects having a low TMB. This data indicates subjects having various cancers and an intermediate TMB (TMB-I) or higher (TMB-H) can be responsive to treatment with SRA737 in combination with LDG. In some cases, TMB-High corresponds to about 20 or more somatic mutations per megabase (Muts/Mb). In some cases, TMB-Intermediate corresponds to about 6 to about 19 Muts/Mb. In certain instances, TMB-Low corresponds to about 5 or less Muts/Mb.

Determining whether a subject will benefit from Chk1 inhibitor therapy can be achieved via comparison of the subject's TMB value to a reference TMB value, e.g., a cut-off value representative of an intermediate TMB level versus a low TMB level. The reference TMB value can be based on the cut-off value that separates a first subset of subjects in a reference population from a second subset of subjects in the reference population based on a significant difference in a subject's responsiveness to treatment with a Chk1 inhibitor (e.g., as described herein). The first subset of subjects can be characterized as having a low TMB and being non-responsive to treatment. The second subset of subjects can be characterized as having an intermediate or high TMB and being responsive to treatment. The reference TMB value represents the cut-off TMB value for subjects having an intermediate TMB rather than low TMB, which distinguishes the first and second subsets of subjects in the reference population. It is understood that the reference TMB value may vary depending on the method used to measure TMB in cancer cells of the subject.

In some cases, the reference TMB value is about 6 somatic mutations per megabase (Muts/Mb), as determined using a FoundationOne assay (e.g., as described herein).

In certain embodiments, the method further includes obtaining, e.g., directly or indirectly, a sample (e.g., a tumor sample or a sample derived from a tumor) from the subject and evaluating the sample for the mutation load or TMB, as described herein. In some cases, the TMB is based on somatic alterations in a predetermined set of genes.

In certain embodiments, the determination of the level of a somatic alteration in the predetermined set of genes set forth in Table 1 comprises a determination of the level of a somatic alteration in about 25 or more, e.g., about 50 or more, about 100 or more, about 150 or more, about 200 or more, about 250 or more, about 260 or more, about 270 or more, about 280 or more, about 290 or more, about 300 or more, about 310 or more, or all genes set forth in Table 1. In certain embodiments, the predetermined set of genes assessed (e.g., in the FoundationOne assay) is 500 or less, such as 450 or less, 400 or less or 350 or less.

In some embodiments, the determination of the level of a somatic alteration in the predetermined set of genes set forth in Table 1 includes a determination of the number of a somatic alteration per a preselected unit, e.g., per megabase in the coding regions of the predetermined set of genes, e.g., in the coding regions of the predetermined set of genes sequenced.

TABLE 1
Exemplary Genes for targeted TMB assessment.
See e.g., FoundationOne
CDx ™ assay described in the experimental section.

| ABL1 | FGF10 | NFKBIA |
|---|---|---|
| ABL2 | FGF14 | NKX2-1 |
| ACVR1B | FGF19 | NOTCH1 |
| AKT1 | FGF23 | NOTCH2 |
| AKT2 | FGF3 | NOTCH3 |
| AKT3 | FGF4 | NPM1 |
| ALK | FGF6 | NRAS |
| ALOX12B | FGFR1 | NSD1 |
| AMER1(FAM123B) | FGFR2 | NTRK1 |
| APC | FGFR3 | NTRK2 |
| AR | FGFR4 | NTRK3 |
| ARAF | FH | NUP93 |
| ARFRP1 | FLCN | PAK3 |
| ARID1A | FLT1 | PALB2 |
| ARID1B | FLT3 | PARK2 |
| ARID2 | FLT4 | PAX5 |
| ASXL1 | FOXL2 | PBRM1 |
| ATM | FOXP1 | PDCD1LG2 |
| ATR | FRS2 | PDGFRA |
| ATRX | FUBP1 | PDGFRB |
| AURKA | GABRA6 | PDK1 |
| AURKB | GATA1 | PIK3C2B |
| AXIN1 | GATA2 | PIK3CA |
| AXL | GATA3 | PIK3CB |
| BAP1 | GATA4 | PIK3CG |
| BARD1 | GATA6 | PIK3R1 |
| BCL2 | GID4 | PIK3R2 |
| BCL2L1 | (C17orf39) | PLCG2 |
| BCL2L2 | GLI1 | PMS2 |
| BCL6 | GNA11 | POLD1 |
| BCOR | GNA13 | POLE |
| BCORL1 | GNAQ | PPP2R1A |
| BLM | GNAS | PRDM1 |
| BRAF | GPR124 | PREX2 |
| BRCA1 | GRIN2A | PRKAR1A |
| BRCA2 | GRM3 | PRKCI |
| BRD4 | GSK3B | PRKDC |
| BRIP1 | H3F3A | PRSS8 |
| BTG1 | HGF | PTCH1 |
| BTG2 | HNF1A | PTEN |
| BTK | HRAS | PTPN11 |
| C11orf30 | HSD3B1 | QKI |
| (EMSY) | HSP90AA1 | RAC1 |
| CARD11 | IDH1 | RAD50 |
| CBFB | IDH2 | RAD51 |
| CBL | IGF1R | RAF1 |
| CCND1 | IGF2 | RANBP2 |
| CCND2 | IKBKE | RARA |
| CCND3 | IKZF1 | RB1 |
| CCNE1 | IL7R | RBM10 |
| CD274 | INHBA | RET |
| CD79A | INPP4B | RICTOR |
| CD79B | IRF2 | RNF43 |
| CDC73 | IRF4 | ROS1 |
| CDH1 | IRS2 | RPTOR |
| CDK12 | JAK1 | RUNX1 |
| CDK4 | JAK2 | RUNX1T1 |
| CDK6 | JAK3 | SDHA |
| CDK8 | JUN | SDHB |
| CDKN1A | KAT6A | SDHC |
| CDKN1B | (MYST3) | SDHD |
| CDKN2A | KDM5A | SETD2 |
| CDKN2B | KDM5C | SF3B1 |
| CDKN2C | KDM6A | SLIT2 |
| CEBPA | KDR | SMAD2 |
| CHD2 | KEAP1 | SMAD3 |
| CHD4 | KEL | SMAD4 |
| CHEK1 | KIT | SMARCA4 |
| CHEK2 | KLHL6 | SMARCB1 |
| CIC | KMT2A | SMO |
| CREBBP | (MLL) | SNCAIP |
| CRKL | KMT2C | SOCS1 |
| CRLF2 | (MLL3) | SOX10 |
| CSF1R | KMT2D | SOX2 |
| CTCF | (MLL2) | SOX9 |
| CTNNA1 | KRAS | SPEN |
| CTNNB1 | LMO1 | SPOP |
| CUL3 | LRP1B | SPTA1 |
| CYLD | LYN | SRC |
| DAXX | LZTR1 | STAG2 |
| DDR2 | MAGI2 | STAT3 |
| DICER1 | MAP2K1 | STAT4 |
| DNMT3A | MAP2K2 | STK11 |
| DOT1L | MAP2K4 | SUFU |
| EGFR | MAP3K1 | SYK |
| EP300 | MCL1 | TAF1 |
| EPHA3 | MDM2 | TBX3 |
| EPHA5 | MDM4 | TERC |
| EPHA7 | MED12 | TERT (promoter only) |
| EPHB1 | MEF2B | TET2 |
| ERBB2 | MEN1 | TGFBR2 |
| ERBB3 | MET | TNFAIP3 |
| ERBB4 | MITF | TNFRSF14 |
| ERG | MLH1 | TOP1 |
| ERRFI1 | MPL | TOP2A |
| ESR1 | MRE11A | TP53 |
| EZH2 | MSH2 | TSC1 |
| FAM46C | MSH6 | TSC2 |
| FANCA | MTOR | TSHR |
| FANCC | MUTYH | U2AF1 |
| FANCD2 | MYC | VEGFA |
| FANCE | MYCL | VHL |
| FANCF | (MYCL1) | WISP3 |
| FANCG | MYCN | WT1 |
| FANCL | MYD88 | XPO1 |
| FAS | NF1 | ZBTB2 |
| FAT1 | NF2 | ZNF217 |
| FBXW7 | NFE2L2 | ZNF703 |
| Select Rearrangements* | | |
| ALK | ETV4 | MYC |
| BCL2 | ETV5 | NOTCH2 |
| BCR | ETV6 | NTRK1 |
| BRAF | FGFR1 | NTRK2 |
| BRCA1 | FGFR2 | PDGFRA |
| BRCA2 | FGFR3 | RAF1 |
| BRD4 | KIT | RARA |
| EGFR | MSH2 | RET |
| ETV1 | MYB | ROS1 |
| | | TMPRSS2 |

*Select rearrangements refers to genes with select intronic regions for the detection of gene rearrangements, one gene with a promoter region and one non-coding RNA gene.

In certain embodiments, a determination that the number of a somatic alteration in the predetermined set of genes is about 5 or less (e.g., 4.5 or less, 4 or less, 3.5 or less, 3 or less) somatic alterations per megabase in the coding regions of the predetermined set of genes set forth in Table 1 indicates that the subject is, or is likely to be, a partial responder or non-responder to the therapy.

In certain embodiments, a determination that the number of somatic alterations in the predetermined set of genes set forth in Table 1 is between about 6 and about 19, e.g., between about 7 and about 19, between about 8 and about 19, or between about 10 and about 19 somatic alterations per megabase in the coding regions of a predetermined set of genes selected from those genes set forth in Table 1, indicates that the subject is, or is likely to be, a partial responder (or will partially respond, or will likely partially respond) to the therapy.

In some embodiments of the method, an intermediate TMB is determined by comparing a TMB value determined from a sample from the subject to a reference TMB value indicative of responsiveness to Chk1 inhibitor therapy. In some cases, the reference TMB value is about 5 or more (e.g., about 5.5 or more, about 6 or more, about 6.5 or more, about 7 or more, about 8 or more, about 9 or more, about 10 or more, about 15 or more, about 20 or more, about 25 or more, about 30 or more, about 35 or more, about 40 or more, about 45 or more, or about 50 or more) somatic alterations per megabase (Mb) in the coding regions of the predetermined set of genes. In certain instances, the reference TMB value is about 6 somatic alterations per megabase (Mb) of coding sequence.

In some embodiments of the method, the reference TMB value is a reference range of TMB values representative of tumor cells from a plurality of subjects having cancer; and the subject is categorized as one who will benefit from Chk1 inhibitor therapy when the determined TMB value from the sample is in the 35th percentile or greater (e.g., 40^{th} percentile or greater, 45^{th} percentile or greater, 50^{th} percentile or greater) of the reference range of TMB values. In certain instances, the three categories of low, intermediate and high TMB are determined based on a distribution of TMB values across a population of subjects of interest. In some cases, the cutoff between low and intermediate TMB is determined to be at about the 33^{rd} percentile of the distribution of TMB values. In some cases, the cutoff between intermediate and TMB is determined to be at about the 66^{th} percentile of the distribution of TMB values. In certain instances, a subject having an intermediate TMB value that is at or below the median or mean TMB of the distribution is selected for treatment according to the subject methods. Accordingly, the method can further include determining a reference range or distribution of TMB values from a plurality of tumor cells samples obtained from a plurality of subjects having cancer.

Methods

Disclosed herein are methods of inhibiting tumor growth in a subject, e.g., a human, by administration of the Chk1 inhibitor SRA737. A detailed description of the compounds, kits comprising the compounds, and methods of use thereof are found below.

Tumor Inhibition

The present disclosure is directed to methods using an effective amount of the compound SRA737 to inhibit the progression of, reduce the size in aggregation of, reduce the volume of, and/or otherwise inhibit the growth of a tumor. Also provided herein are methods of treating the underlying disease, e.g., cancer, and extending the survival of the subject.

In some aspects, provided for is a method of inhibiting the growth of a tumor in a subject in need thereof, the method comprising administering to the subject an effective amount of SRA737. In some aspects, the disclosure provides for a method of administering to the subject an effective amount of SRA737 to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by tumor volume. In some aspects, the disclosure provides for a method of administering to the subject an effective amount of SRA737 to inhibit growth of a tumor, wherein tumor growth is reduced by 1%, 2%, 3%, 4%, 5%, 6%, 7%, 8%, 9%, 1%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the absolute size of the tumor. In some aspects, the disclosure provides for a method of administering to the subject an effective amount of SRA737 to inhibit the growth of a tumor, wherein tumor growth is reduced by 1%, 2%, 3%, 4%, 5%, 6%, 7%, 8%, 9%, 10%, %12%, 14%, 16%, 18%, 20%, 22%, 24%, 26%, 28%, 30%3, 32%, 34%, 36%3, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the expression levels of tumor markers for that type of tumor.

In some aspects, provided for is a method of treating a cancer, comprising administering to a subject with the cancer an effective amount of a SRA737 compound. In some aspects, provided for is a method of treating a cancer, comprising administering to a subject with the cancer an effective amount of a SRA737 compound, wherein the method results in a regression of a tumor. The regression, in general, is determined relative to a baseline measurement. The regression can be a partial regression or a complete regression. The regression can, in general, be measured by any assay useful for quantitating size, volume, and/or growth of a tumor, e.g., medical imaging techniques known in the art. The regression can be a 1%, 2%, 3%, 4%, 5%, 6%, 7%, 8%, 9%, 1%, 12%, 14, 16%, %18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by tumor volume. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the absolute size of the tumor. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the expression levels of tumor markers for that type of tumor. The regression can be a 30% regression. The regression can be a 30% regression as measured by any assay useful for quantitating size, volume, and/or growth of a tumor, e.g., medical imaging techniques known in the art.

The present disclosure is also directed to methods using an effective amount of the compound SRA737 and a second effective amount of a further treatment to inhibit the progression of, reduce the size in aggregation of, reduce the volume of, and/or otherwise inhibit the growth of a tumor. Also provided herein are methods of treating the underlying disease, e.g., cancer, and extending the survival of the subject. In some aspects, provided for is a method of inhibiting the growth of a tumor in a subject in need thereof, the method comprising administering to the subject an effective amount of SRA737 and a second effective amount of a further treatment. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by tumor volume. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the absolute size of the tumor. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the expression levels of tumor markers for that type of tumor.

In some aspects, provided for is a method of treating a cancer, comprising administering to a subject with the cancer an effective amount of a SRA737 compound and a second effective amount of a further treatment. In some aspects, provided for is a method of treating a cancer, comprising administering to a subject with the cancer an effective amount of a SRA737 compound and a second effective amount of a further treatment, wherein the method results in a regression of a tumor. The regression, in general, is determined relative to a baseline measurement. The regression can be a partial regression or a complete regression. The regression can, in general, be measured by any assay useful for quantitating size, volume, and/or growth of a tumor, e.g., medical imaging techniques known in the art. The regression can be a 1%, 2%, 3%, 4%, 5%, 6%, 7%, 8%, 9%, 1%, 2%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by tumor volume. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the absolute size of the tumor. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the expression levels of tumor markers for that type of tumor. The regression can be a 30% regression. The regression can be a 30% regression as measured by any assay useful for quantitating size, volume, and/or growth of a tumor, e.g., medical imaging techniques known in the art.

Types of Tumors

In some aspects, the present disclosure provides for methods of inhibiting the growth of a tumor wherein the tumor is from a cancer that is colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

Accordingly, the present disclosure also provides for methods of treating a cancer in a subject in need thereof, the method comprising administering an effective amount of SRA737 to the subject. In some aspects, methods are disclosed for the treatment of cancer wherein the cancer is colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

In certain embodiments of the subject method, the cancer is selected from colon, colorectal, endometrial, esophageal, lung, mesothelioma, and prostate.

In certain embodiments of the subject method, the cancer is a squamous cell carcinoma. In certain embodiments of the subject method, the cancer is selected from advanced/metastatic squamous cell carcinoma of the anus, penis, vagina, and vulva. In certain embodiments of the subject method, the cancer is selected from anogenital, rectal, ovarian, and cervical.

In some embodiments of the subject method, the cancer is anogenital cancer. In certain instances, the cancer is anal. In some cases, the cancer is rectal. In some cases, the cancer is cervical. In some cases, the cancer is squamous cervical.

In some embodiments of the subject method, the cancer is ovarian. In some cases, the ovarian cancer is high-grade serous ovarian cancer (HGSOC).

In certain instances of any one of the cancers described above, the cancer is positive for human papillomavirus (HPV).

Clinical Endpoints

Provided herein are methods for inhibiting the growth of a tumor in a subject and/or cell, wherein the conditions of said methods are such that the method results in a clinically relevant endpoint.

Tumor growth occurs when one or more biological cells grow and divide much more rapidly resulting in an increase in the number of cells in comparison to the normal and healthy process of cells division. This phenomenon is an indication that the cells are in a disease state such as cancer or pre-cancer. Moreover, tumor growth oftentimes comes about in discrete stages prior to the agglomerated cells forming a tumor.

There are several methods the skilled artisan can use to measure cell replication rates. The overall metabolic activity inside a cell can be measured via a labeled biological product. For example, there are several commercially available dyes (e.g. MTT) that can penetrate the cell and interact with certain enzymes and other factors to produce a detectable product. Also, cellular biomarkers can be measured in a cell. For example, a BrdU assay can incorporate a thymidine derivative into cellular DNA and be detected with an antibody. Proliferating cell nuclear antigen (PCNA) is another such biomarker for detection. Besides tagging techniques, the skilled artisan can also use for example, microscopy or flow cytometry to allow for cell counts.

In one aspect, cellular replication is measured by a clinical endpoint that includes: a quality of life (QOL) score, duration of response (DOR, clinical benefit rate (CBR), patient reported outcomes (PRO), an objective response rate (ORR) score, a disease-free survival (DFS) or progression-free survival (PFS), a time to progression (TTP), an Overall Survival (OS), a time-to-treatment failure (TTF), RECIST criteria, and/or a Complete Response. the clinical endpoints can be determined using methods well known to one of skill in the art.

In some aspects, the present disclosure provides methods wherein the growth of the tumor is reduced no more than 5, 10, 20, 40, 50, 60, 80, 90, 95, 97, 99, or 99.9% after administration of the effective amount of SRA737.

In some aspects, the present disclosure provides methods wherein the % reduction is calculated based on measurement(s) of one or more clinical endpoints.

In some aspects, the present disclosure provides methods wherein the growth of the tumor is reduced as measured by an increase or a decrease in total cell count in a MTT assay, or by change in genetic profile as measured by a ctDNA assay, by no more than or at least 5, 10, 20, 30, 40, 50, 60, 70, 80, 90, 95, 97, 99, or 99.9% after administration of the effective amount of SRA737.

In some aspects, the present disclosure provides methods wherein the growth of the tumor is reduced at least 5, 10, 20, 30, 40, 50, 60, 70, 80, 90, 95, 97, 99, or 99.9% after administration of the effective amount of SRA737. In some aspects, the present disclosure provides methods wherein the growth of the tumor is reduced as measured by an increase or a decrease in total cell count in a MTT assay, or by change in genetic profile as measured by a ctDNA assay, by at least 5, 10, 20, 30, 40, 50, 60, 70, 80, 90, 95, 97, 99, or 99.9% after administration of the effective amount of SRA737.

In some aspects, the present disclosure provides methods wherein administration results in an IC₅₀ value below 10 μM and/or a GI₅₀ value below 1 μM. In some aspects, the present disclosure provides methods wherein administration results in an IC₅₀ value below 10 μM and/or a GI₅₀ value below 1 μM at twenty-four (24) hours after administration. In some aspects, the present disclosure provides methods wherein administration results in an IC₅₀ value below 10 μM and/or a GI₅₀ value below 1 μM at forty-eight (48) hours after administration.

In some aspects, the present disclosure provides methods wherein the administration results in an AUC of at least 1, 10, 25, 50, 100, 200, 400, 600, 800, or 1000.

In some aspects, the present disclosure provides methods wherein the administration results in an IC₅₀ value of no more than 0.001, 0.005, 0.01, 0.05, 0.1, 1, 3, 5, 10, 20, 40, 50, 60, 80, 90, 100, 200, 250, 300, 350, or 400 μM.

In some aspects, the present disclosure provides methods wherein the administration results in an EC₅₀ value of at least 0.01, 0.1, 1, 3, 5, 10, 20, 40, 50, 60, 80, 90, 100, 200, 250, 300, 350, or 400 μM.

In some aspects, the present disclosure provides methods wherein the administration results in an therapeutic index (TI) value ranging from about 1.001:1 to about 50:1, about 1.1:1 to about 15:1, about 1.2:1 to about 12:1, about 1.2:1 to about 10:1, about 1.2:1 to about 5:1, or about 1.2:1 to about 3:1.

In some aspects, the present disclosure provides methods wherein the administration results in an GI₅₀ value of at least 0.1 μM, 0.3 μM, 0.5 μM, 0.7 μM, 1 μM, 1.5 μM, 2 μM, 2.5 μM, 3 μM, 4 μM, 5 μM, or 10 μM.

In some aspects, the present disclosure provides methods wherein the administration results in a Maximum Response Observed (Max Response) value of no more than 0.1, 0.5, 1, 2 μM, 2.5 μM, 3 μM, 4 μM, 5 μM, or 10 μM.

Tumor growth can be expressed in terms of total tumor volume or total tumor size. There exist formulas, both generally speaking and specific to certain tumor models, that the skilled artisan can use to calculate tumor volume based upon the assumption that solid tumors are more or less spherical. In this regard, the skilled artisan can use experimental tools such as: ultrasound imaging, manual or digital calipers, ultrasonography, computed tomographic (CT), microCT, ¹⁸F-FDG-microPET, or magnetic resonance imaging (MRI) to measure tumor volume. See for example Monga S P, Wadleigh R, Sharma A, et al. Intratumoral therapy of cisplatin/epinephrine injectable gel for palliation in patients with obstructive esophageal cancer. Am. J. Clin. Oncol. 2000; 23(4):386-392; Mary M. Tomayko C., Patrick Reynolds, 1989. Determination of subcutaneous tumor size in athymic (nude) mice. Cancer Chemotherapy and Pharmacology, Volume 24, Issue 3, pp 148-154; E Richtig, G Langmann, K Müllner, G Richtig and J Smolle, 2004. Calculated tumour volume as a prognostic parameter for survival in choroidal melanomas. Eye (2004) 18, 619-623; Jensen et al. BMC Medical Imaging 2008. 8:16; Tomayko et al. Cancer Chemotherapy and Pharmacology September 1989, Volume 24, Issue 3, pp 148-154; and Faustino-Rocha et al. Lab Anim (NY). 2013 June; 42(6):217-24, each of which are hereby incorporated by reference in their entirety. In an illustrative example, tumor growth and/or size can be measured as a sum of the diameters (longest for non-nodal lesions, short axis for nodal lesions) for all target lesions and can be, in general, calculated and reported as the baseline sum diameters. The baseline sum diameters can be, in general, used as reference to further characterize any objective tumor regression in a measurable dimension of the disease.

In some aspects, the present disclosure provides methods wherein administration results in a reduction in tumor size, of at least 5, 10, 20, 30, 40, 50, 60, 70, 80, 90, 95, 97, 99 or 99.9% after administration of the effective amount of SRA737. In some aspects, the present disclosure provides methods wherein administration results in a reduction in tumor size of at least 30% after administration of the effective amount of SRA737. In some aspects, the present disclosure provides methods wherein administration results in a reduction in tumor volume of at least 5, 10, 20, 30, 40, 50, 60, 70, 80, 90, 95, 97, 99 or 99.9% after administration of the effective amount of SRA737. In some aspects, the present disclosure provides methods wherein administration results in a reduction in tumor volume of at least 30% after administration of the effective amount of SRA737. In some aspects, the present disclosure provides methods wherein administration results in a reduction in tumor volume or tumor size after one (1), two (2), three (3), four (4), six (6), eight (8), twelve (12), sixteen (16), twenty (20), twenty four (24), thirty six (36), or fifty two (52) weeks. In some aspects, the present disclosure provides methods wherein administration results in a reduction in tumor volume or tumor size of at least 30% after one (1), two (2), three (3), four (4), six (6), eight (8), twelve (12), sixteen (16), twenty (20), twenty four (24), thirty six (36), or fifty two (52) weeks. Reductions in tumor volume or tumor size can be measured by medical imaging techniques. Reductions in tumor volume or tumor size are, in general, determined relative to a baseline measurement.

Subjects

The present disclosure provides for administering an effective amount of SRA737 to a subject that is in need thereof, including subjects identified as having a genetic abnormality or biomarker of interest (e.g., as described herein). The present disclosure provides for administering an effective amount of SRA737 in a combination therapy with a further treatment to a subject that is in need thereof. In some aspects, the tumor from a subject is screened with genetic testing and/sequencing prior to administration. In some aspects, the tumor from a subject is screened with genetic testing and/sequencing after administration. In some aspects, the tumor from a subject is screened both after and before administration. In some aspects, healthy cells from the subject are screened with genetic testing and/sequencing prior to administration, after administration, or both. In some aspects, the tumor from a subject is screened with other biological tests or assays to determine the level of expression of certain biomarkers. In some aspects, the tumor from a subject is screened with both genetic testing and/sequencing and other biomarker tests or assays.

In some aspects, the present disclosure provides for methods wherein the subject is a mammal. In some aspects, the present disclosure provides for methods wherein the subject is a primate.

In some aspects, the present disclosure provides for methods wherein the subject is a mouse.

In some aspects, the present disclosure provides for methods wherein the subject is a human.

In some aspects, the present disclosure provides for methods wherein the subject is a human that has a tumor having a genetic mutation in one or more of the following genes: a tumor suppressor gene, a DNA damage repair gene, a replication stress gene, or an oncogenic driver gene. In some aspects, the present disclosure provides for methods wherein the subject is suffering from cancer in which the cancer cells have a genetic mutation in one or more of the following genes: a tumor suppressor gene, a DNA damage repair gene, a replication stress gene, or an oncogenic driver gene.

In some aspects, the present disclosure provides for methods wherein the tumor is in a human suffering from cancer that is selected from the group consisting of: colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer. In certain embodiments, the tumor is in a human suffering from cancer that is selected from colon, colorectal, endometrial, esophageal, lung, mesothelioma, and prostate.

In certain embodiments, the tumor is in a human suffering from cancer that is a squamous cell carcinoma. In certain embodiments, the tumor is in a human suffering from cancer that is selected from advanced/metastatic squamous cell carcinoma of the anus, penis, vagina, and vulva. In certain embodiments, the tumor is in a human suffering from cancer that is selected from anogenital, rectal, ovarian, and cervical. In some embodiments, the tumor is in a human suffering from anogenital cancer. In certain instances, the cancer is anal. In some cases, the cancer is rectal. In some cases, the cancer is cervical. In some cases, the cancer is squamous cervical. In some embodiments, the tumor is in a human suffering from cancer that is ovarian. In some cases, the ovarian cancer is high-grade serous ovarian cancer (HGSOC).

In some aspects, subjects have:
a. Histologically or cytologically proven advanced malignancy of the following types, for which no other conventional therapy is considered appropriate:
   i. High-grade serous ovarian cancer (HGSOC)
      1. Histologically confirmed high-grade serous ovarian, fallopian tube, or primary peritoneal cancer
      2. Platinum-resistant or refractory disease, or if the subject is intolerant to platinum therapy
   ii. Small cell lung cancer
      1. Subjects, in general, have received at least 1 but no more than 3 prior regimens for advanced disease, unless otherwise approved by sponsor
   iii. Soft tissue sarcoma
      1. Including undifferentiated pleiomorphic sarcoma/malignant fibrous histiocytoma (MFH) (including high-grade spindle cell sarcoma/pleomorphic liposarcomas), leiomyosarcoma, and dedifferentiated liposarcomas. Other types of STS may be eligible with sponsor's approval
      2. Subjects, in general, have received at least 1 but no more than 3 prior regimens for advanced disease, unless otherwise approved by sponsor
   iv. Cervical/anogenital cancer
      1. Including all cervical carcinoma and advanced/metastatic squamous cell carcinoma of the anus, penis, vagina, and vulva
      2. Subjects, in general, have received at least 1 but no more than 3 prior regimens for advanced disease, unless otherwise approved by sponsor
   v. Urothelial Carcinoma
      1. Histologically confirmed locally advanced and unresectable or metastatic urothelial carcinoma of the bladder, upper urinary tract or urethra
      2. Subjects, in general, have received at least 1 but no more than 3 prior regimens for advanced disease.
b. Measurable disease per RECIST v1.1 (see below)
c. Subjects have predicted sensitivity to Chk1 inhibition based on factors including: genetic profiling of tumor tissue or ctDNA, HPV status, and germline BRCA1 and BRCA2 gene status. All subjects have genetic profiling from tumor tissue or ctDNA; profiling is performed prospectively if required to evaluate Chk1 sensitivity or otherwise performed retrospectively
   i. For subjects with HGSOC, documented somatic or germline BRCA1 and BRCA2 wild-type status confer eligibility without requirement for prospective genetic profiling. If documented BRCA status is not available, genetic profiling may be performed prospectively to determine eligibility.
   ii. Subjects with SCLC are eligible without requirement for prospective genetic profiling on the basis of very high prevalence of cancer related alterations in the tumor suppressor genes (eg, TP53 and RB1) in this population.
   iii. For subjects with STS, and any others for whom genetic profiling is performed prospectively, eligibility is determined by the sponsor's review of genetic abnormalities detected in genes in the following categories:
      Key tumor suppressor genes regulating G1 cell cycle progression/arrest such as RB1, TP53, etc. For relevant cancers, positive human papilloma virus (HPV) status is also considered for eligibility.
         a. The DNA damage response pathway including ATM, BRCA1, BRCA2, mismatch repair genetic alterations and/or high microsatellite instability.
         b. Genetic indicators of replicative stress such as gain of function/amplification of Chk1 or ATR or other related gene.
         c. Oncogenic drivers such as MYC, CCNE1, etc.
   iv. For subjects with anogenital cancer, known HPV positive status will confer eligibility without requirement for prospective genetic profiling. If HPV status is not known or not positive, genetic profiling (or HPV testing where appropriate) may be performed prospectively to determine eligibility. Subjects with cervical cancer or squamous cell carcinoma of the anus are eligible without requirement for prospective genetic profiling based on the very high prevalence of HPV positivity in these populations.

In some aspects, subjects have one of the histologically or cytologically proven advanced malignancies described above and tumor tissue or ctDNA evidence that their tumor harbors one or more mutations that are expected to confer sensitivity to Chk1 inhibition. Eligibility can be determined by the sponsor's review of genetic abnormalities detected in genes in the following categories:
a. Key tumor suppressor genes regulating G1 cell cycle progression/arrest such as RB1, TP53, etc. For relevant cancers, positive human papilloma virus (HPV) status is also considered for eligibility.
b. The DNA damage response pathway including ATM, BRCA1, BRCA2, mismatch repair genetic alterations and/or high microsatellite instability.
c. Genetic indicators of replicative stress such as gain of function/amplification of Chk1 or ATR or other related gene.
d. Oncogenic drivers such as MYC, KRAS, etc.

In some aspects, subjects are excluded based on the following criteria:
a. Received the following prior or current anticancer therapy in the timeframes noted prior to receiving SRA737 and have recovered from toxicity:
   i. Radiotherapy, chemotherapy, PARP inhibitors, other targeted therapies, or other IMPs within 2 weeks
   ii. Nitrosoureas or Mitomycin C within 6 weeks
   iii. Any prior treatment with a Chk1 inhibitor at any point or prior treatment with an ATR inhibitor within 6 months
b. No more than 3 previous treatment regimens for advanced disease (not applicable to HGSOC expansion cohort)
c. Other malignancy within the past 2 years, except for adequately treated tumors
d. If, in the opinion of the Investigator, the subject is highly likely to experience clinically significant myelosuppression
e. Ongoing toxic manifestations of previous treatments greater than NCI-CTCAE Grade 1
f. History of allergy to gemcitabine
g. New or progressing brain metastases. Subjects with brain metastases that have been asymptomatic and radiologically stable over an 8-week period and have not been treated with steroids during that time may be included with approval from the sponsor.
h. High medical risk because of nonmalignant systemic disease
i. Serologically positive for hepatitis B, hepatitis C or HIV
j. Serious cardiac condition, left ventricular ejection fraction <45% at baseline, history of cardiac ischemia within the past 6 months, or prior history of cardiac arrhythmia requiring treatment, unless approved by the sponsor.
k. Prior bone marrow transplant or extensive radiotherapy to greater than 25% of bone marrow within the previous 8 weeks
l. Peanut allergy
m. QTcF>450 msec in adult makes and >470 msec in adult females
n. Impairment of gastrointestinal (GI) function or GI disease that may significantly alter the absorption of SRA737
o. Inability to swallow capsules without chewing or crushing
p. Is a participant or plans to participate in another interventional clinical trial
q. Any other condition which in the Investigator's opinion would not make the subject a good candidate

Administration

As disclosed herein, the methods of the invention include administration of the effective amount of SRA737. In an embodiment, the effective amount of SRA737 is administered as a monotherapy.

Also disclosed herein, the methods of the invention include a combination therapy administering an effective amount of SRA737 and coadministering a second effective amount of a further treatment. Further treatments include, but are not limited to, administering a chemotherapeutic agent, administering an antibody or antibody fragment (such as an immune checkpoint inhibitors), administering a radiation treatment, administering an external inducer of replication stress, and administering a combination thereof. Further treatments also include, but are not limited to, administering any one of gemcitabine, olaparib, niraparib, rucaparib, talazoparib, cisplatin, a ribonucleotide reductase inhibitor, etoposide, SN-38/CPT-11, mitomycin C, and combinations thereof. Coadministered encompasses methods where SRA737 and the further treatment are given simultaneously, where SRA737 and the further treatment are given sequentially, and where either one of, or both of, SRA737 and the further treatment are given intermittently or continuously, or any combination of: simultaneously, sequentially, intermittently and/or continuously. The skilled artisan will recognize that intermittent administration is not necessarily the same as sequential because intermittent also includes a first administration of an agent and then another administration later in time of that very same agent. Moreover, the skilled artisan understands that intermittent administration also encompasses sequential administration in some aspects because intermittent administration does include interruption of the first administration of an agent with an administration of a different agent before the first agent is administered again. Further, the skilled artisan will also know that continuous administration can be accomplished by a number of routes including i.v. drip or feeding tubes, etc.

Furthermore, and in a more general way, the term “coadministered” encompasses any and all methods where the individual administration of SRA737 and the individual administration of the further treatment to a subject overlap during any timeframe.

In one aspect, the frequency of administration of SRA737 or the further treatment to a subject includes, but is not limited to, Q1d, Q2d, Q3d, Q4d, Q5d, Q6d, Q7d, Q8d, Q9d, Q10d, Q14d, Q21d, Q28d, Q30d, Q90d, Q120d, Q240d, or Q365d. The term “QnD or qnd” refers to drug administration once every “n” days. For example, QD (or qd) refers to once every day or once daily dosing, Q2D (or q2d) refers to a dosing once every two days, Q7D refers to a dosing once every 7 days or once a week, Q5D refers to dosing once every 5 days, and so on. In one aspect, SRA737 and the further treatment are administered on different schedules.

In another aspect, the frequency of administration of SRA737 or the further treatment to a subject includes, but is not limited to: 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; twice daily dosing; or dosing on days 2 and 3 of a weekly cycle. In one aspect, SRA737 and the further treatment are administered on different schedules.

In one aspect, the present disclosure provides for methods where either one of or both of or any combination thereof SRA737 and/or the further treatment are administered intermittently. In one aspect, the present disclosure provides for methods comprising administering either one of, or both of, or any combinations thereof, SRA737 or the further treatment, to a subject with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations. In such aspects, the administration with a delay follows a pattern where one of or both of or any combination thereof SRA737 and/or the further treatment are administered continuously for a given period of time from about ten (10) minutes to about three hundred and sixty five (365) days and then is not administered for a given period of time from about ten (10) minutes to about thirty (30) days. In one aspect, the present disclosure provides for methods where either one of or any combination of SRA737 and/or the further treatment are administered intermittently while the other is given continuously.

In one aspect, the present disclosure provides for methods where the combination of the effective amount of SRA737 is administered sequentially with the second effective amount of a further treatment.

In one aspect, the present disclosure provides for methods where SRA737 and the further treatment are administered simultaneously. In one aspect, the present disclosure provides for methods where the combination of the effective amount of SRA737 is administered sequentially with the second effective amount of a further treatment. In such aspects, the combination is also said to be “coadministered” since the term includes any and all methods where the subject is exposed to both components in the combination. However, such aspects are not limited to the combination being given just in one formulation or composition. In some cases, certain concentrations of SRA737 and the further treatment are more advantageous to deliver at certain intervals and as such, the effective amount of SRA737 and the second effective amount of the further treatment may change according to the formulation being administered.

In some aspects, the present disclosure provides for methods wherein SRA737 and the further treatment are administered simultaneously or sequentially. In some aspects, the present disclosure provides for methods where the effective amount of SRA737 is administered sequentially after the second effective amount of the further treatment. In some aspects, the present disclosure provides for methods where the second effective amount of the further treatment is administered sequentially after the effective amount of SRA737.

In some aspects, the present disclosure provides for methods where the combination is administered in one formulation. In some aspects, the present disclosure provides for methods where the combination is administered in two (2) compositions where the effective amount of SRA737 is administered in a separate formulation from the formulation of the second effective amount of the further treatment.

In some aspects, the present disclosure provides for methods where the effective amount of SRA737 is administered sequentially after the second effective amount of the further treatment. In some aspects, the present disclosure provides for methods where the second effective amount of the further treatment is administered sequentially after the effective amount of SRA737. In some aspects, the SRA737 and the further treatment are administered; and subsequently both SRA737 and the further treatment are administered intermittently for at least twenty-four (24) hours. In some aspects, SRA737 and the further treatment are administered on a non-overlapping every other day schedule. In some aspects, the further treatment is administered on day 1, and SRA737 is administered on days 2 and 3 of a weekly schedule.

In some aspects, the present disclosure provides for methods where the effective amount of SRA737 is administered no less than four (4) hours after the second effective amount of the further treatment. In one aspect, the present disclosure provides for methods where the effective amount of SRA737 is administered no less than ten (10) minutes, no less than fifteen (15) minutes, no less than twenty (20) minutes, no less than thirty (30) minutes, no less than forty (40) minutes, no less than sixty (60) minutes, no less than one (1) hour, no less than two (2) hours, no less than four (4) hours, no less than six (6) hours, no less than eight (8) hours, no less than ten (10) hours, no less than twelve (12) hours, no less than twenty four (24) hours, no less than two (2) days, no less than four (4) days, no less than six (6) days, no less than eight (8) days, no less than ten (10) days, no less than twelve (12) days, no less than fourteen (14) days, no less than twenty one (21) days, or no less than thirty (30) days after the second effective amount of the further treatment. In one aspect, the present disclosure provides for methods where the second effective amount of the further treatment is administered no less than ten (10) minutes, no less than fifteen (15) minutes, no less than twenty (20) minutes, no less than thirty (30) minutes, no less than forty (40) minutes, no less than sixty (60) minutes, no less than one (1) hour, no less than two (2) hours, no less than four (4) hours, no less than six (6) hours, no less than eight (8) hours, no less than ten (10) hours, no less than twelve (12) hours, no less than twenty four (24) hours, no less than two (2) days, no less than four (4) days, no less than six (6) days, no less than eight (8) days, no less than ten (10) days, no less than twelve (12) days, no less than fourteen (14) days, no less than twenty one (21) days, or no less than thirty (30) days after the effective amount of a SRA737.

In some aspects, the present disclosure provides for methods where either one of, or both of, or any combination thereof, SRA737 and/or a further treatment are administered by a route selected from the group consisting of: intravenous, subcutaneous, cutaneous, oral, intramuscular, and intraperitoneal. In some aspects, the present disclosure provides for methods where either one of, or both of, or any combination thereof, SRA737 and/or a further treatment are administered intravenously. In some aspects, the present disclosure provides for methods where either one of, or both of, or any combination thereof, SRA737 and/or a further treatment are administered orally.

It is understood by the skilled artisan that the unit dose forms of the present disclosure may be administered in the same or different physicals forms, i.e. orally via capsules or tablets and/or by liquid via i.v. infusion, and so on. Moreover, the unit dose forms for each administration may differ by the particular route of administration. Several various dosage forms may exist for either one of, or both of, SRA737 and a further treatment. Because different medical conditions can warrant different routes of administration, the same components of a combination of SRA737 and a further treatment described herein may be exactly alike in composition and physical form and yet may need to be given in differing ways and perhaps at differing times to alleviate the condition. For example, a condition such as persistent nausea, especially with vomiting, can make it difficult to use an oral dosage form, and in such a case, it may be necessary to administer another unit dose form, perhaps even one identical to other dosage forms used previously or afterward, with an inhalation, buccal, sublingual, or suppository route instead or as well. The specific dosage form may be a requirement for certain combinations of SRA737 and a further treatment, as there may be issues with various factors like chemical stability or pharmacokinetics.

Therapeutically Effective Amount and Unit Dose Form

The present disclosure provides for a method of treatment wherein the effective amount of SRA737 is administered to a subject. The term “effective amount” or “therapeutically effective amount” refers to an amount that is effective to ameliorate a symptom of a disease, e.g. an amount that is effective to inhibit the growth of a tumor. In some aspects, the effective amount of SRA737 is less than or equal to the maximum tolerated dose (MTD), less than or equal to the highest non-severely toxic dose (HNSTD), or less than or equal to the No-observed-adverse-effect-level (NOAEL). In some aspects, the effective amount of SRA737 is less than 2000 mg/day orally. In some aspects, the effective amount of SRA737 is less than 1500 mg/day orally. In some aspects, the effective amount of SRA737 is less than 1300 mg/day orally. In some aspects, the effective amount of SRA737 is greater than 600 mg/day orally. In some aspects, the effective amount of SRA737 is between 600-2000 mg/day orally. In some aspects, the effective amount of SRA737 is between 600-1500 mg/day orally. In some aspects, the effective amount of SRA737 is between 600-1300 mg/day orally. In some aspects, the effective amount of SRA737 is between 600-1000 mg/day orally. In some aspects, the effective amount of SRA737 is 600 mg/day, 700 mg/day, 800 mg/day, 900 mg/day, 1000 mg/day, 1100 mg/day, 1200 mg/day, 1300 mg/day, 1500 mg/day, or 2000 mg/day orally.

In specific embodiments of the invention, the effective amount of SRA737 is administered to a subject as a monotherapy. In some aspects, the effective amount of the SRA737 monotherapy is less than or equal to the maximum tolerated dose (MTD), less than or equal to the highest non-severely toxic dose (HNSTD), or less than or equal to the No-observed-adverse-effect-level (NOAEL). In some aspects, the effective amount of the SRA737 monotherapy is less than 2000 mg/day orally. In some aspects, the effective amount of the SRA737 monotherapy is less than 1500 mg/day orally. In some aspects, the effective amount of the SRA737 monotherapy is less than 1300 mg/day orally. In some aspects, the effective amount of the SRA737 monotherapy is greater than 600 mg/day orally. In some aspects, the effective amount of the SRA737 monotherapy is between 600-2000 mg/day orally. In some aspects, the effective amount of the SRA737 monotherapy is between 600-1500 mg/day orally. In some aspects, the effective amount of the SRA737 monotherapy is between 600-1300 mg/day orally. In some aspects, the effective amount of the SRA737 monotherapy is between 600-1000 mg/day orally. In some aspects, the effective amount of the SRA737 monotherapy is 600 mg/day, 700 mg/day, 800 mg/day, 900 mg/day, 1000 mg/day, 1100 mg/day, 1200 mg/day, 1300 mg/day, 1500 mg/day, or 2000 mg/day orally. In some aspects, the effective amount of the SRA737 monotherapy is 600 mg/day. In some aspects, the effective amount of the SRA737 monotherapy is 700 mg/day. In some aspects, the effective amount of the SRA737 monotherapy is 800 mg/day. In some aspects, the effective amount of the SRA737 monotherapy is 900 mg/day. In some aspects, the effective amount of the SRA737 monotherapy is 1000 mg/day. In some aspects, the effective amount of the SRA737 monotherapy is 1100 mg/day. In some aspects, the effective amount of the SRA737 monotherapy is 1200 mg/day. In some aspects, the effective amount of the SRA737 monotherapy is 1300 mg/day. In some aspects, the effective amount of the SRA737 monotherapy is 1500 mg/day. In some aspects, the effective amount of the SRA737 monotherapy is or 2000 mg/day.

In specific embodiments of the invention, the effective amount of SRA737 is administered to a subject as a combination therapy. In some aspects, the effective amount of the SRA737 combination therapy is less than or equal to the maximum tolerated dose (MTD), less than or equal to the highest non-severely toxic dose (HNSTD), or less than or equal to the No-observed-adverse-effect-level (NOAEL). In some aspects, the effective amount of the SRA737 combination therapy is less than the effective amount of the SRA737 monotherapy. In some aspects, the effective amount of the SRA737 combination therapy is less than 2000 mg/day orally. In some aspects, the effective amount of the SRA737 combination therapy is less than 1500 mg/day orally. In some aspects, the effective amount of the SRA737 combination therapy is less than 1300 mg/day orally. In some aspects, the effective amount of the SRA737 combination therapy is 600 mg/day or less orally. In some aspects, the effective amount of the SRA737 combination therapy is at least 300 mg/day orally. In some aspects, the effective amount of the SRA737 combination therapy is at least 100 mg/day orally. In some aspects, the effective amount of the SRA737 combination therapy is at least 600 mg/day orally. In some aspects, the effective amount of the SRA737 combination therapy is between 100-2000 mg/day orally. In some aspects, the effective amount of the SRA737 combination therapy is between 300-2000 mg/day orally. In some aspects, the effective amount of the SRA737 combination therapy is between 600-2000 mg/day orally. In some aspects, the effective amount of the SRA737 combination therapy is between 300-1500 mg/day orally. In some aspects, the effective amount of the SRA737 combination therapy is between 300-1300 mg/day orally. In some aspects, the effective amount of the SRA737 combination therapy is between 300-1000 mg/day orally. In some aspects, the effective amount of the SRA737 combination therapy is 100 mg/day, 150 mg/day, 200 mg/day, 300 mg/day, 600 mg/day, 700 mg/day, 800 mg/day, 900 mg/day, 1000 mg/day, 1100 mg/day, 1200 mg/day, 1300 mg/day, 1500 mg/day, or 2000 mg/day orally. In some aspects, the effective amount of the SRA737 combination therapy is 300 mg/day, 400 mg/day, 500 mg/day, 600 mg/day, 700 mg/day, 800 mg/day, 900 mg/day, 1000 mg/day, 1100 mg/day, 1200 mg/day, 1300 mg/day, 1500 mg/day, or 2000 mg/day orally.

In some aspects, the effective amount of the SRA737 combination therapy is 300 mg/day. In some aspects, the effective amount of the SRA737 combination therapy is 400 mg/day. In some aspects, the effective amount of the SRA737 combination therapy is 500 mg/day. In some aspects, the effective amount of the SRA737 combination therapy is 600 mg/day. In some aspects, the effective amount of the SRA737 combination therapy is 700 mg/day. In some aspects, the effective amount of the SRA737 combination therapy is 800 mg/day. In some aspects, the effective amount of the SRA737 combination therapy is 900 mg/day. In some aspects, the effective amount of the SRA737 combination therapy is 1000 mg/day. In some aspects, the effective amount of the SRA737 combination therapy is 1100 mg/day. In some aspects, the effective amount of the SRA737 combination therapy is 1200 mg/day. In some aspects, the effective amount of the SRA737 combination therapy is at least 300 mg/day. In some aspects, the effective amount of the SRA737 combination therapy is at least 400 mg/day. In some aspects, the effective amount of the SRA737 combination therapy is at least 500 mg/day. In some aspects, the effective amount of the SRA737 combination therapy is at least 600 mg/day. In some aspects, the effective amount of the SRA737 combination therapy is at least 700 mg/day. In some aspects, the effective amount of the SRA737 combination therapy is at least 800 mg/day. In some aspects, the effective amount of the SRA737 combination therapy is at least 900 mg/day. In some aspects, the effective amount of the SRA737 combination therapy is at least 1000 mg/day. In some aspects, the effective amount of the SRA737 combination therapy is at least 1100 mg/day. In some aspects, the effective amount of the SRA737 combination therapy is at least 1200 mg/day. In some aspects, the effective amount of the SRA737 combination therapy is 300 mg/day or less. In some aspects, the effective amount of the SRA737 combination therapy is 400 mg/day or less. In some aspects, the effective amount of the SRA737 combination therapy is 500 mg/day or less. In some aspects, the effective amount of the SRA737 combination therapy is 600 mg/day or less. In some aspects, the effective amount of the SRA737 combination therapy is 700 mg/day or less. In some aspects, the effective amount of the SRA737 combination therapy is 800 mg/day or less. In some aspects, the effective amount of the SRA737 combination therapy is 900 mg/day or less. In some aspects, the effective amount of the SRA737 combination therapy is 1000 mg/day or less. In some aspects, the effective amount of the SRA737 combination therapy is 1100 mg/day or less. In some aspects, the effective amount of the SRA737 combination therapy is 1200 mg/day or less.

In some aspects, the effective amount of the SRA737 combination therapy is 350 mg/day. In some aspects, the effective amount of the SRA737 combination therapy is 450 mg/day. In some aspects, the effective amount of the SRA737 combination therapy is 550 mg/day. In some aspects, the effective amount of the SRA737 combination therapy is 650 mg/day. In some aspects, the effective amount of the SRA737 combination therapy is 750 mg/day. In some aspects, the effective amount of the SRA737 combination therapy is 850 mg/day. In some aspects, the effective amount of the SRA737 combination therapy is 950 mg/day. In some aspects, the effective amount of the SRA737 combination therapy is 1050 mg/day. In some aspects, the effective amount of the SRA737 combination therapy is 1150 mg/day. In some aspects, the effective amount of the SRA737 combination therapy is 1250 mg/day. In some aspects, the effective amount of the SRA737 combination therapy is at least 350 mg/day. In some aspects, the effective amount of the SRA737 combination therapy is at least 450 mg/day. In some aspects, the effective amount of the SRA737 combination therapy is at least 550 mg/day. In some aspects, the effective amount of the SRA737 combination therapy is at least 650 mg/day. In some aspects, the effective amount of the SRA737 combination therapy is at least 750 mg/day. In some aspects, the effective amount of the SRA737 combination therapy is at least 850 mg/day. In some aspects, the effective amount of the SRA737 combination therapy is at least 950 mg/day. In some aspects, the effective amount of the SRA737 combination therapy is at least 1050 mg/day. In some aspects, the effective amount of the SRA737 combination therapy is at least 1150 mg/day. In some aspects, the effective amount of the SRA737 combination therapy is at least 1250 mg/day. In some aspects, the effective amount of the SRA737 combination therapy is 350 mg/day or less. In some aspects, the effective amount of the SRA737 combination therapy is 450 mg/day or less. In some aspects, the effective amount of the SRA737 combination therapy is 550 mg/day or less. In some aspects, the effective amount of the SRA737 combination therapy is 650 mg/day or less. In some aspects, the effective amount of the SRA737 combination therapy is 750 mg/day or less. In some aspects, the effective amount of the SRA737 combination therapy is 850 mg/day or less. In some aspects, the effective amount of the SRA737 combination therapy is 950 mg/day or less. In some aspects, the effective amount of the SRA737 combination therapy is 1050 mg/day or less. In some aspects, the effective amount of the SRA737 combination therapy is 1150 mg/day or less. In some aspects, the effective amount of the SRA737 combination therapy is 1250 mg/day or less.

In some aspects, the effective amount of the SRA737 combination therapy is 325 mg/day. In some aspects, the effective amount of the SRA737 combination therapy is 425 mg/day. In some aspects, the effective amount of the SRA737 combination therapy is 525 mg/day. In some aspects, the effective amount of the SRA737 combination therapy is 625 mg/day. In some aspects, the effective amount of the SRA737 combination therapy is 725 mg/day. In some aspects, the effective amount of the SRA737 combination therapy is 825 mg/day. In some aspects, the effective amount of the SRA737 combination therapy is 925 mg/day. In some aspects, the effective amount of the SRA737 combination therapy is 1025 mg/day. In some aspects, the effective amount of the SRA737 combination therapy is 1125 mg/day. In some aspects, the effective amount of the SRA737 combination therapy is 1225 mg/day. In some aspects, the effective amount of the SRA737 combination therapy is at least 325 mg/day. In some aspects, the effective amount of the SRA737 combination therapy is at least 425 mg/day. In some aspects, the effective amount of the SRA737 combination therapy is at least 525 mg/day. In some aspects, the effective amount of the SRA737 combination therapy is at least 625 mg/day. In some aspects, the effective amount of the SRA737 combination therapy is at least 725 mg/day. In some aspects, the effective amount of the SRA737 combination therapy is at least 825 mg/day. In some aspects, the effective amount of the SRA737 combination therapy is at least 925 mg/day. In some aspects, the effective amount of the SRA737 combination therapy is at least 1025 mg/day. In some aspects, the effective amount of the SRA737 combination therapy is at least 1125 mg/day. In some aspects, the effective amount of the SRA737 combination therapy is at least 1225 mg/day. In some aspects, the effective amount of the SRA737 combination therapy is 325 mg/day or less. In some aspects, the effective amount of the SRA737 combination therapy is 425 mg/day or less. In some aspects, the effective amount of the SRA737 combination therapy is 525 mg/day or less. In some aspects, the effective amount of the SRA737 combination therapy is 625 mg/day or less. In some aspects, the effective amount of the SRA737 combination therapy is 725 mg/day or less. In some aspects, the effective amount of the SRA737 combination therapy is 825 mg/day or less. In some aspects, the effective amount of the SRA737 combination therapy is 925 mg/day or less. In some aspects, the effective amount of the SRA737 combination therapy is 1025 mg/day or less. In some aspects, the effective amount of the SRA737 combination therapy is 1125 mg/day or less. In some aspects, the effective amount of the SRA737 combination therapy is 1225 mg/day or less.

In some aspects, the effective amount of the SRA737 combination therapy is 375 mg/day. In some aspects, the effective amount of the SRA737 combination therapy is 475 mg/day. In some aspects, the effective amount of the SRA737 combination therapy is 575 mg/day. In some aspects, the effective amount of the SRA737 combination therapy is 675 mg/day. In some aspects, the effective amount of the SRA737 combination therapy is 775 mg/day. In some aspects, the effective amount of the SRA737 combination therapy is 875 mg/day. In some aspects, the effective amount of the SRA737 combination therapy is 975 mg/day. In some aspects, the effective amount of the SRA737 combination therapy is 1075 mg/day. In some aspects, the effective amount of the SRA737 combination therapy is 1175 mg/day. In some aspects, the effective amount of the SRA737 combination therapy is 1275 mg/day. In some aspects, the effective amount of the SRA737 combination therapy is at least 375 mg/day. In some aspects, the effective amount of the SRA737 combination therapy is at least 475 mg/day. In some aspects, the effective amount of the SRA737 combination therapy is at least 575 mg/day. In some aspects, the effective amount of the SRA737 combination therapy is at least 675 mg/day. In some aspects, the effective amount of the SRA737 combination therapy is at least 775 mg/day. In some aspects, the effective amount of the SRA737 combination therapy is at least 875 mg/day. In some aspects, the effective amount of the SRA737 combination therapy is at least 975 mg/day. In some aspects, the effective amount of the SRA737 combination therapy is at least 1075 mg/day. In some aspects, the effective amount of the SRA737 combination therapy is at least 1175 mg/day. In some aspects, the effective amount of the SRA737 combination therapy is at least 1275 mg/day. In some aspects, the effective amount of the SRA737 combination therapy is 375 mg/day or less. In some aspects, the effective amount of the SRA737 combination therapy is 475 mg/day or less. In some aspects, the effective amount of the SRA737 combination therapy is 575 mg/day or less. In some aspects, the effective amount of the SRA737 combination therapy is 675 mg/day or less. In some aspects, the effective amount of the SRA737 combination therapy is 775 mg/day or less. In some aspects, the effective amount of the SRA737 combination therapy is 875 mg/day or less. In some aspects, the effective amount of the SRA737 combination therapy is 975 mg/day or less. In some aspects, the effective amount of the SRA737 combination therapy is 1075 mg/day or less. In some aspects, the effective amount of the SRA737 combination therapy is 1175 mg/day or less. In some aspects, the effective amount of the SRA737 combination therapy is 1275 mg/day or less.

In specific embodiments of the invention, the effective amount of SRA737 is administered to a subject as a combination therapy with a second effective amount of a further treatment. In some aspects, the second effective amount is an amount from about 0.001 mg/kg to about 15 mg/kg. In some embodiments the second effective amount of the further treatment is 0.001, 0.005, 0.010, 0.020, 0.050, 0.1, 0.2, 0.5, 1.0, 2.0, 5.0, 10.0 or 15.0 mg/kg. In some embodiments the second effective amount of the further treatment is between 10-2000 mg/m²/day. In some embodiments the second effective amount of the further treatment is between 50-1250 mg/m²/day. In some embodiments the second effective amount of the further treatment is 50 mg/m²/day, 100 mg/m²/day, 150 mg/m²/day, 200 mg/m²/day, 250 mg/m²/day, 300 mg/m²/day, 350 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 850 mg/m²/day, 900 mg/m²/day, 950 mg/m²/day, 1000 mg/m²/day, 1050 mg/m²/day, 1100 mg/m²/day, 1150 mg/m²/day, 1200 mg/m²/day, or 1250 mg/m²/day.

In general, the compounds of the present technology will be administered in a therapeutically effective amount by any of the accepted modes of administration for agents that serve similar utilities. the actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the present technology, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the maximum tolerated dose (MTD), the highest non-severely toxic dose (HNSTD), the No-observed-adverse-effect-level (NOAEL), or the LD₅₀ (the dose lethal to 50% of the population) and the ED₅₀ (the dose therapeutically effective in 50% of the population). the dose ratio of toxic to therapeutic effects is therapeutic index, which can be expressed as the ratio of the MTD, HNSTD, NOAEL, or LD₅₀ to the ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. the exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). the MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

A therapeutically effective amount can be the same or different than either one of, or both of, the effective amount of SRA737 and the second effective amount of the further treatment. This is because the present disclosure provides that the methods, as described herein, are effective even where neither the effective amount of SRA737 nor the second effective amount of the further treatment must be an amount that, alone, will ameliorate a symptom of a disease (e.g., the amount of the SRA737 and/or the further treatment may be considered a “sub-therapeutic” amount if administered as an individual therapy). However, the present disclosure does provide that a therapeutically effective amount of the combination must be provided, i.e. the combination does at least affect a treatment of a symptom of a disease.

A unit dose form is a term that is generally understood by the skilled artisan. A unit dose forms is a pharmaceutical drug product that is marketed for a specific use. The drug product includes the active ingredient(s) and any inactive components, most often in the form of pharmaceutically acceptable carriers or excipients. It is understood that multiple unit dose forms are distinct drug products. Accordingly, one unit dose form may be e.g. the combination of SRA737 and a further treatment of 250 mg at a certain ratio of each component, while another completely distinct unit dose form is e.g. the combination of SRA737 and a further treatment of 750 mg at the same certain ratio of each component referred to above. So from one unit dose form to another, the effective amount of SRA737 and the second effective amount of the further treatment may both remain the same. Of course, when the either one of the effective amount of SRA737 or the second effective amount of the further treatment changes, the unit dose form is distinct.

In some aspects, the effective amount is unique to the SRA737 compound, i.e. it is different than the second effective amount of the further treatment. In some aspects, the effective amount of SRA737 is an amount that is equivalent to a “therapeutically effective amount” or an amount that brings about a therapeutic and/or beneficial effect. In some aspects, the effective amount of SRA737 is a “therapeutically effective amount”. In some aspects, the second effective amount of the further treatment is a “therapeutically effective amount”. In some aspects, both the effective amount of SRA737 and second effective amount of the further treatment are not a “therapeutically effective amount”. In some aspects, the second effective amount is unique to the of the further treatment, i.e. the second effective amount is a different amount for different further treatments.

In some aspects, the SRA737 and the further treatment combination is formulated in one (1) unit dose form. In some aspects, the same unit dose form is administered for at least four (4) hours, six (6) hours, eight (8) hours, twelve (12) hours, twenty four (24) hours, one (1) day, two (2) days, three (3) days, seven (7) days, ten (10) days, fourteen (14) days, twenty one (21) days, or thirty (30) days.

In some aspects, the SRA737 and the further treatment combination is formulated in at least two (2) separately distinct unit dose forms. In some aspects, the first effective amount is different in the first unit dose form than in the second unit dose form. In some aspects, the effective amount of SRA737 is the same in the first unit dose form as it is in the second unit dose form.

In some aspects, the first unit dose form is the same as the second unit dose form. In some aspects, the first unit dose form is the same as the second and third unit dose forms. In some aspects, the first unit dose form is the same as the second, third, and fourth unit dose forms.

Compounds of the Invention

In one aspect, the present disclosure provides for methods of use of the compound SRA737.

SRA737

The compound SRA737 is also identified by the chemical name: 5-[[4-[[morpholin-2-yl]methylamino]-5-(trifluoromethyl)-2-pyridyl]amino]pyrazine-2-carbonitrile. Each of the enantiomers of SRA737 is useful for compositions, methods and kits disclosed herein.

SRA737 is a compound that is disclosed in international patent application no. PCT/GB2013/051233, which is herein incorporated by reference. The skilled artisan will find the how to synthesize SRA737 in international patent application no. PCT/GB2013/051233.

In one aspect, the SRA737 structures are as shown in the table below.

TABLE 1A
SRA737 Structure

| Description | Structure |
|---|---|
| SRA737 structure | 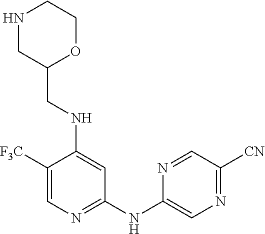 [SMILES: N#Cc1cnc(Nc2cc(NCC3CNCCO3)c(C(F)(F)F)cn2)cn1] |

Combination Therapies

In another aspect, the present disclosure provides for methods of use of the compound SRA737 in a combination therapy with a further treatment.

Further treatments include, but are not limited to, administering a chemotherapeutic agent, administering an antibody or antibody fragment (such as an immune checkpoint inhibitor), administering a radiation treatment, administering an external inducer of replication stress, and administering a combination thereof.

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, nab-paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), and vinca alkaloids (e.g., vincristine, vindesine, vinorelbine, and vinblastine). Chemotherapeutic agents includes DNA intercalating agents (e.g., pyrrolobenzodiazepines).

The term “external inducer of replication stress” refers to any agent that causes increased stalled replication forks, increased genomic instability, increased mutation and/or mutation rate, activation of DNA damage repair pathways, activation of the DNA damage response (DDR), activation or increased expression of replication stress gene(s), or combinations thereof. Examples of inducers of replication stress include, but are not limited to, genotoxic chemotherapeutic agents (e.g., gemcitabine and other nucleoside analogs, alkylating agents such as temozolomide, cisplatin, mitomycin C and others, topoisomerase inhibitors such as camptothecin and etoposide and others). External inducers of cell stress include agents that reduce the concentration of nucleotides in a cell (e.g., ribonucleotide reductase inhibitors and the like). External inducers of cell stress include agents also include PARP inhibitors.

The term “DNA damage repair (DDR) gene” or “DNA damage repair pathway gene” refers to any gene that directly or indirectly promotes repair of DNA mutations, breaks or other DNA damage or structural changes. DNA damage repair genes include, but are not limited to, the following genes: ATM, CDK12, BRCA1, BRCA2, MRE11A, ATR, and Rad50. DDR genes also include genes in the Fanconi anemia (FA) pathway. Genes in the FA pathway include, but are not limited to, Fanconi anemia complementation group (FANC) genes.

The term “immune checkpoint inhibitor” refers to binding molecules that bind to and block or inhibit the activity of one or more immune checkpoint molecules or drugs that inhibit immunosuppressive proteins. Illustrative immune checkpoints inhibitors include antibodies, or antigen binding fragments thereof, that target one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, CGEN-15049, and IDO1.

The term “PARP inhibitor” or “PARPi” refers to an inhibitor of PARP. A PARPi may be a small molecule, an antibody or a nucleic acid. A PARPi may function to reduce the expression of PARP or the activity of PARP in cells, or combinations thereof. PARPi include inhibitors that do or do not alter the binding of PARP to DNA. PARPi may inhibit any members of the PARP family. PARPi include, but are not limited to: Olaparib, Rucaparib, Veliparib, Niraparib, Iniparib, Talazoparib, Veliparib, Fluzoparib, BGB-290, CEP-9722, BSI-201, EZ449, PF-01367338, AZD2281, INO-1001, MK-4827, SC10914, and 3-aminobenzamine.

In specific aspects, further treatments include, but are not limited to, administering any one of gemcitabine, olaparib, niraparib, rucaparib, talazoparib, cisplatin, a ribonucleotide reductase inhibitor, etoposide, SN-38/CPT-11, mitomycin C, and combinations thereof.

Pharmaceutical Compositions

Methods for inhibiting the growth of a tumor, inhibiting the progression of or treating cancer are described herein. Said methods of the invention include administering an effective amount of SRA737 and a second effective amount of a further treatment. the SRA737 and the further treatment can each be formulated in pharmaceutical compositions. these pharmaceutical compositions may comprise, in addition to the active compound(s), a pharmaceutically acceptable excipient, carrier, buffer, stabilizer or other materials well known to those skilled in the art. Such materials should be non-toxic and should not interfere with the efficacy of the active ingredient. the precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin. Liquid pharmaceutical compositions generally include a liquid carrier such as water or oil, including oils of petroleum, animal, vegetable or synthetic origin, e.g., peanut oil, soybean oil, mineral oil, sesame oil, etc. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

A composition can be administered alone or in combination with other treatments, either simultaneously or sequentially dependent upon the condition to be treated.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. there are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject's inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present technology in combination with at least one pharmaceutically acceptable excipient. Acceptable excipients are non-toxic, aid administration, and do not adversely affect therapeutic benefit of the claimed compounds. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including oils of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the present technology in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

In some embodiments, the pharmaceutical compositions include a pharmaceutically acceptable salt. The term “pharmaceutically acceptable salt” refers to salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in Stahl and Wermuth (Eds.), Handbook of Pharmaceutical Salts Properties, Selection, and Use; 2002.

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. the pack or dispenser device may be accompanied by instructions for administration. Compositions comprising a compound of the present technology formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of the present technology based on the total formulation, with the balance being one or more suitable pharmaceutical excipients. Preferably, the compound is present at a level of about 1-80 wt %. Representative pharmaceutical formulations are described below.

### Formulation Examples

The following are representative pharmaceutical formulations containing the SRA737 and a further treatment, either alone or in combination.

A composition can be administered alone or in combination with other treatments, either simultaneously or sequentially dependent upon the condition to be treated.

Kits

The present disclosure also provides for a kit comprising the combination of SRA737 and a further treatment and instructions for use. The present disclosure further provides for a kit comprising one or more pharmaceutical compositions where the pharmaceutical composition(s) comprise SRA737 and a further treatment, and instructions for use, optionally the combination includes at least one pharmaceutically acceptable carrier or excipient.

Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the antigen-binding construct.

In some aspects, the disclosure provides for a kit comprising a combination of SRA737 and a further treatment and at least one pharmaceutically acceptable carrier or excipient.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

In another aspect described herein, an article of manufacture containing materials useful for the treatment, prevention and/or diagnosis of the disorders described herein, e.g., inhibition of tumor growth is provided. The article of manufacture comprises a container and a label or package insert on or associated with the container. Suitable containers include, for example, bottles, vials, syringes, iv. solution bags, etc. The containers may be formed from a variety of materials such as glass or plastic. The container(s) holds a composition which is by itself or combined with another composition effective for treating, preventing and/or diagnosing the disorder and may have a sterile access port (for example the container may be an intravenous solution bag or a vial having a stopper pierceable by a hypodermic injection needle).

The article of manufacture in this embodiment described herein may further comprise a label or package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Polypeptides and Nucleic Acids

Described herein are polypeptide and nucleic acid sequences of genes useful for the invention, e.g., genes for CHK1. In some embodiments, polypeptide and nucleic acid sequences useful for the invention are at least 95, 96, 97, 98, or 99% identical to sequences described herein or referred to herein by a database accession number. In some embodiments, polypeptide and nucleic acid sequences useful for the invention are 100% identical to sequences described herein or referred to herein by a database accession number.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Definitions

Terms used in the claims and specification are defined as set forth herein unless otherwise specified.

The practice of the present invention includes the use of conventional techniques of organic chemistry, molecular biology (including recombinant techniques), microbiology, cell biology, biochemistry and immunology, which are within the skill of the art.

In this application, reference will be made to a number of technical designations. All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about.” Reagents described herein are exemplary and equivalents of such may be known in the art.

Compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²⁵I), or carbon-14 (¹⁴C). All isotopic variations of the compounds of the present invention, whether radioactive or not, are intended to be encompassed within the scope of the present invention.

The term “subject” refers to any mammal including humans, and mammals such as those animals of veterinary and research interest that are including, but not limited to: simians, cattle, horses, dogs, cats, and rodents. Animals such as mice and rats, and other mammals, can be used in screening, characterization, and evaluation of medicaments. As used herein, the terms patient, subject and individual are used interchangeably.

The term “administering” or “administration of” a drug and/or therapy to a subject (and grammatical equivalents of this phrase) refers to both direct or indirect administration, which may be administration to a subject by a medical professional, may be self-administration, and/or indirect administration, which may be the act of prescribing or inducing one to prescribe a drug and/or therapy to a subject.

The term “coadministration” refers to two or more compounds administered in a manner to exert their pharmacological effect during the same period of time. Such coadministration can be achieved by either simultaneous, contemporaneous, or sequential administration of the two or more compounds.

The term “treating” or “treatment of” a disorder or disease refers to taking steps to alleviate the symptoms of the disorder or disease, e.g., tumor growth or cancer, or otherwise obtain some beneficial or desired results for a subject, including clinical results. Any beneficial or desired clinical results may include, but are not limited to, alleviation or amelioration of one or more symptoms of cancer or conditional survival and reduction of tumor load or tumor volume; diminishment of the extent of the disease; delay or slowing of the tumor progression or disease progression; amelioration, palliation, or stabilization of the tumor and/or the disease state; or other beneficial results.

The term “in situ” or “in vitro” refers to processes that occur in a living cell growing separate from a living organism, e.g., growing in tissue culture.

The term “in vivo” refers to processes that occur in a living organism.

The term “Chk1” or “CHEK1” or “checkpoint kinase 1” refers to serine/threonine-protein kinase that is encoded by the CHEK1 gene.

The term “effective amount” means an amount sufficient to produce a desired effect, e.g., an amount sufficient to inhibit tumor growth.

The term “reduction” of a symptom or symptoms (and grammatical equivalents of this phrase) refers to decreasing the severity or frequency of the symptom(s), or elimination of the symptom(s).

Notwithstanding the appended claims, the disclosure is also defined by the following clauses:

Clause 1. A method of treating a cancer, comprising administering to a subject with the cancer an effective amount of a SRA737 compound, wherein the effective amount is less than 2000 mg/day.

Clause 2. The method of clause [00212], wherein the SRA737 compound is administered orally.

Clause 3. The method of any of clauses [00212]-0, wherein the SRA737 compound is administered daily.

Clause 4. The method of clause 0, wherein the SRA737 compound is administered for at least 28 consecutive days.

Clause 5. The method of clause 0, wherein the SRA737 compound is administered for at least 7 consecutive days.

Clause 6. The method of clauses [00212] or 0, wherein the SRA737 compound is administered intermittently.

Clause 7. The method of clause 0, wherein the SRA737 compound is administered with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations.

Clause 8. The method of any of clauses [00212]-0, wherein the SRA737 compound is administered over one or more 28 day cycles.

Clause 9. The method of clause 0, wherein the SRA737 compound is administered on one or more days of the one or more 28 day cycles.

Clause 10. The method of clause 0, wherein the SRA737 compound is administered on days 2, 3, 9, 10, 16, and 17 of the one or more 28 day cycles.

Clause 11. The method of clause 0-0, further comprising administering an initial dose of the SRA737 compound prior to the first of the one or more 28 day cycles.

Clause 12. The method of clause 0, wherein the initial dose is administered 4 days, 5 days, 6 days, or 7 days prior to the first cycle of the one or more 28 day cycles.

Clause 13. The method of any one of clauses 0-0, wherein the one or more 28 day cycles comprises 2, 3, 4, 5, 6 or more 28 day cycles.

Clause 14. The method of any of clauses [00212]-0, wherein the SRA737 compound is administered following a dosing schedule selected from the group consisting of: 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; and dosing on days 2 and 3 of a weekly cycle.

Clause 15. The method of any of clauses [00212]-0, wherein the effective amount is administered in a single dose once a day.

Clause 16. The method of any of clauses [00212]-0, wherein half of the effective amount is administered twice a day.

Clause 17. The method of any of clauses [00212]-0, wherein the effective amount is less than 1500 mg/day.

Clause 18. The method of any of clauses [00212]-0, wherein the effective amount is less than 1300 mg/day.

Clause 19. The method of any of clauses [00212]-0, wherein the effective amount is 1000 mg/day or less.

Clause 20. The method of any of clauses [00212]-0, wherein the effective amount is 900 mg/day or less.

Clause 21. The method of any of clauses [00212]-0, wherein the effective amount is 800 mg/day or less.

Clause 22. The method of any of clauses [00212]-0, wherein the effective amount is 700 mg/day or less.

Clause 23. The method of any of clauses [00212]-0, wherein the effective amount is 600 mg/day or less.

Clause 24. The method of any of clauses [00212]-0, wherein the effective amount is 500 mg/day or less.

Clause 25. The method of any of clauses [00212]-0, wherein the effective amount is 400 mg/day or less.

Clause 26. The method of any of clauses [00212]-0, wherein the effective amount is between 600 mg/day and 1300 mg/day.

Clause 27. The method of any of clauses [00212]-0, wherein the effective amount is between 300 mg/day and 1300 mg/day.

Clause 28. The method of any of clauses [00212]-0, wherein the effective amount is between 300 mg/day and 1000 mg/day.

Clause 29. The method of any of clauses [00212]-0, wherein the effective amount is between 300 mg/day and 800 mg/day.

Clause 30. The method of any of clauses [00212]-0, wherein the effective amount is between 500 mg/day and 1300 mg/day.

Clause 31. The method of any of clauses [00212]-0, wherein the effective amount is between 500 mg/day and 1000 mg/day.

Clause 32. The method of any of clauses [00212]-0, wherein the effective amount is between 500 mg/day and 800 mg/day.

Clause 33. The method of any of clauses [00212]-0, wherein the effective amount is selected from the group consisting of: 600 mg/day, 700 mg/day, 800 mg/day, 900 mg/day, 1000 mg/day, 1100 mg/day, and 1200 mg/day.

Clause 34. The method of any of clauses [00212]-0, wherein the effective amount is selected from the group consisting of: 40 mg/day, 80 mg/day, 300 mg/day, 500 mg/day, 600 mg/day, 700 mg/day, and 800 mg/day.

Clause 35. The method of any of clauses [00212]-0, wherein the effective amount is 300 mg/day.

Clause 36. The method of any of clauses [00212]-0, wherein the effective amount is 400 mg/day.

Clause 37. The method of any of clauses [00212]-0, wherein the effective amount is 500 mg/day.

Clause 38. The method of any of clauses [00212]-0, wherein the effective amount is 600 mg/day.

Clause 39. The method of any of clauses [00212]-0, wherein the effective amount is 700 mg/day.

Clause 40. The method of any of clauses [00212]-0, wherein the effective amount is 800 mg/day.

Clause 41. The method of any of clauses [00212]-0, wherein the effective amount is 900 mg/day.

Clause 42. The method of any of clauses [00212]-0, wherein the effective amount is 1000 mg/day.

Clause 43. The method of any of clauses [00212]-[00253], wherein the cancer is metastatic cancer.

Clause 44. The method of any of clauses [00212]-[00253], wherein the cancer is a condition or disorder selected from the group consisting of: colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

Clause 45. The method of clause [00212]-[00253], wherein the cancer is colorectal cancer.

Clause 46. The method of clause [00256], wherein the colorectal cancer is characterized as having a microsatellite instability or a deficiency in mismatch repair (MMR).

Clause 47. The method of clause [00212]-[00253], wherein the cancer is non-small cell lung cancer.

Clause 48. The method of clause [00212]-[00253], wherein the cancer is HNSCC.

Clause 49. The method of clause [00212]-[00253], wherein the cancer is SCCA.

Clause 50. The method of clause [00212]-[00253], wherein the cancer is anogenital cancer.

Clause 51. The method of clause [00212]-[00253], wherein the cancer is prostate cancer.

Clause 52. The method of clause [00262], wherein the prostate cancer is metastatic castration-resistant prostate cancer (mCRPC).

Clause 53. The method of clause [00212]-[00253], wherein the cancer is ovarian cancer.

Clause 54. The method of clause [00264], wherein the ovarian cancer is high-grade serous ovarian cancer (HGSOC).

Clause 55. The method of clause [00265], wherein a tumor associated with the HGSOC is identified as having an increased expression of a Cyclin E1 (CCNE) gene.

Clause 56. The method of clause [00266], wherein the increased expression is a result of genetic amplification.

Clause 57. The method of clause [00265], wherein the tumor is identified as having somatic or germline BRCA1 and BRCA2 wild-type status.

Clause 58. The method of any of clauses [00212]-[00268], wherein a tumor associated with the cancer is identified as having a gain of function mutation, amplification or overexpression of at least one oncogenic driver gene or other gene implicated in Chk1 pathway sensitivity.

Clause 59. The method of clause [00269], wherein the oncogenic driver gene is selected from the group consisting of: MYC, MYCN, KRAS, and CCNE1.

Clause 60. The method of any of clauses [00212]-[00270], wherein a tumor associated with the cancer is identified as having a loss of function or a deleterious mutation in at least one DNA damage repair (DDR) pathway gene implicated in Chk1 pathway sensitivity.

Clause 61. The method of clause [00271], wherein the DDR pathway gene is selected from the group consisting of: ATM, CDK12, BRCA1, BRCA2, MRE11A, ATR, and an FA pathway gene.

Clause 62. The method of clause [00271] or [00272], wherein the loss of function or the deleterious mutation is determined by establishing microsatellite instability or a deficiency in mismatch repair (MMR).

Clause 63. The method of any of clauses [00212]-[00273], wherein a tumor associated with the cancer is identified as having a gain of function mutation or amplification of at least one replication stress gene implicated in Chk1 pathway sensitivity.

Clause 64. The method of clause [00274], wherein the replication stress gene is ATR or CHK1.

Clause 65. The method of any of clauses [00212]-[00275], wherein a tumor associated with the cancer is identified as having a deleterious mutation in a tumor suppressor (TS) gene implicated in Chk1 pathway sensitivity.

Clause 66. The method of clause [00276], wherein a tumor associated with the cancer suppressor gene is selected from the group consisting of: RB1, TP53, ATM, RAD50, FBXW7 and PARK2.

Clause 67. The method of any of clauses [00212]-[00277], wherein the subject is human papillomavirus (HPV) positive.

Clause 68. The method of any of clauses [00212]-[00278], wherein the subject is human.

Clause 69. The method of any of clauses [00212]-[00279], further comprising administering a second effective amount of a further treatment, wherein the further treatment is selected from the group consisting of: a chemotherapeutic agent, an antibody or antibody fragment, a radiation treatment, an external inducer of replication stress, and a combination thereof.

Clause 70. The method of clause [00280], wherein the further treatment is selected from the group consisting of: gemcitabine, olaparib, niraparib, rucaparib, talazoparib, cisplatin, a ribonucleotide reductase inhibitor, etoposide, SN-38/CPT-11, mitomycin C, and combinations thereof.

Clause 71. The method of clause [00280], wherein the further treatment comprises gemcitabine.

Clause 72. The method of any of clauses [00280]-[00282], wherein the further treatment is administered daily.

Clause 73. The method of any of clauses [00280]-[00282], wherein the further treatment is administered on day 1 and the SRA737 compound is administered on days 2 and 3 of a weekly schedule.

Clause 74. The method of any of clauses [00280]-[00282], wherein the further treatment and the SRA737 compound are administered over one or more 28 day cycles.

Clause 75. The method of clause 0, wherein the further treatment is administered on days 1, 8, and 15 of the one or more 28 day cycles, and the SRA737 compound is administered on days 2, 3, 9, 10, 16, and 17 of the one or more 28 day cycles.

Clause 76. The method of any of clauses [00282]-0, wherein the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 77. The method of any of clauses [00282]-0, wherein the second effective amount of the further treatment is 600 mg/m²/day or less.

Clause 78. The method of any of clauses [00282]-0, wherein the second effective amount of the further treatment is between 50 and 600 mg/m²/day.

Clause 79. The method of any of clauses [00282]-0, wherein the second effective amount of the further treatment is between 50 and 300 mg/m²/day.

Clause 80. The method of any of clauses [00282]-0, wherein the effective amount of the SRA737 compound is 80 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 81. The method of any of clauses [00282]-0, wherein the effective amount of the SRA737 compound is 150 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 82. The method of any of clauses [00282]-0, wherein the effective amount of the SRA737 compound is 300 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 83. The method of any of clauses [00282]-0, wherein the effective amount of the SRA737 compound is 500 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 84. The method of any of clauses [00282]-0, wherein the effective amount of the SRA737 compound is 600 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 85. The method of any of clauses [00282]-0, wherein the effective amount of the SRA737 compound is 700 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 86. The method of any of clauses [00282]-0, wherein the effective amount of the SRA737 compound is 800 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 87. The method of any of clauses [00282]-0, wherein the effective amount of the SRA737 compound is 900 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 88. The method of any of clauses [00282]-0, wherein the effective amount of the SRA737 compound is 1000 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

Clause 89. The method of any of clauses [00280]-0, wherein the cancer is urothelial carcinoma.

Clause 90. The method of clause [00300], wherein the urothelial carcinoma is selected from the group consisting of: (a) unresectable urothelial carcinomas of the bladder, upper urinary tract, or urethra, and (b) metastatic urothelial carcinomas of the bladder, upper urinary tract, or urethra.

Clause 91. The method of any of clauses [00280]-0, wherein the cancer is HGSOC.

Clause 92. The method of clause [00302], wherein a tumor associated with the HGSOC is identified as having somatic or germline BRCA1 and BRCA2 wild-type status.

Clause 93. The method of any of clauses [00280]-0, wherein the cancer is small cell lung cancer.

Clause 94. The method of any of clauses [00280]-0, wherein the cancer is soft tissue sarcoma.

Clause 95. The method of clause [00305], wherein the soft tissue sarcoma is selected from the group consisting of: undifferentiated pleiomorphic sarcoma, malignant fibrous histiocytoma (MFH)/high-grade spindle cell sarcoma, pleomorphic liposarcomas, leiomyosarcoma, and dedifferentiated liposarcoma.

Clause 96, The method of any of clauses [00280]-0, wherein the cancer is cervical or anogenital cancer.

Clause 97. The method of clause [00307], wherein the cervical or anogenital cancer is selected from the group consisting of: advanced/metastatic squamous cell carcinoma of the anus, penis, vagina, and vulva.

Clause 98. The method of any of clauses [00212]-[00308], wherein the method results in growth inhibition of a tumor associated with the cancer.

Clause 99. The method of clause [00309], wherein the growth inhibition of the tumor associated with the cancer is a minimum growth inhibition of at least 10%, at least 20%, at least 30%, at least 40%, at least 50%, at least 60%, at least 70%, at least 80%, or at least 90% relative to an untreated tumor.

Clause 100. The method of any of clauses [00212]-[00310], wherein the method results in a regression of a tumor associated with the cancer relative to a baseline measurement.

Clause 101. The method of clause [00311], wherein the regression is a 30% regression of the tumor associated with the cancer relative to the baseline measurement.

Clause 102. The method of clause [00311], wherein the regression is a complete regression of the tumor associated with the cancer relative to the baseline measurement.

Clause 103. The method of any of clauses [00212]-[00313], wherein the method results in cytotoxicity of a tumor associated with the cancer.

Clause 104. The method of any of clauses [00212]-[00314], wherein the method results in a partial response, a complete response, or a stable disease in the subject relative to a baseline measurement.

Clause 105. The method of any of clauses [00212]-[00314], wherein the method results in a partial response in the subject relative to a baseline measurement.

Clause 106. The method of any of clauses [00212]-[00314], wherein the method results in a complete response in the subject relative to a baseline measurement.

Clause 107. The method of any of clauses [00212]-[00314], wherein the method results in a stable disease in the subject relative to a baseline measurement.

Clause 108. The method of any of clauses [00212]-[00318], wherein the method results in a plasma Cₘᵢₙ of at least 100 ng/ml of the SRA737 compound for at least 24 hours in the subject after administration.

Clause 109. The method of any of clauses [00212]-[00318], wherein the method results in a plasma Cₘᵢₙ of at least 100 nM of the SRA737 compound for at least 24 hours in the subject after administration.

Clause 110. The method of any of clauses [00212]-[00320], wherein the method results in a plasma AUC₀₋₂₄ of at least 100 ng·h/mL, at least 300 ng·h/mL, at least 600 ng·h/mL, at least 800 ng·h/mL, at least 1000 ng·h/mL, at least 1600 ng·h/mL, at least 2300 ng·h/mL, at least 2500 ng·h/mL, at least 3000 ng·h/mL, at least 3500 ng·h/mL, at least 8000 ng·h/mL, at least 12000 ng·h/mL, at least 15000 ng·h/mL, at least 18000 ng·h/mL, at least 20000 ng·h/mL, at least 25000 ng·h/mL, or at least 29000 ng·h/mL of the SRA737 compound in the subject after administration.

Clause 111. The method of any of clauses [00212]-[00320], wherein the method results in a plasma AUC₀₋₁₂ of at least 400 ng·h/mL, at least 500 ng·h/mL, at least 600 ng·h/mL, at least 1600 ng·h/mL, at least 2600 ng·h/mL, at least 4500 ng·h/mL, at least 5000 ng·h/mL, at least 8000 ng·h/mL, at least 8000 ng·h/mL, at least 1000 ng·h/mL of the SRA737 compound in the subject after administration.

Clause 112. The method of any of clauses [00212]-[00322], wherein the method results in a plasma Cₘₐₓ of at least 500 ng/mL, at least 600 ng/mL, at least 800 ng/mL, at least 100 ng/mL, at least 150 ng/mL, at least 175 ng/mL, at least 350 ng/mL, at least 990 ng/mL, at least 1980 ng/mL, at least 2000 ng/mL, or at least 3228 ng/mL of the SRA737 compound in the subject after administration.

Clause 113. The method of any of clauses [00212]-[00322], wherein the method results in a plasma Cₘₐₓ of less than 500 ng/mL, less than 600 ng/mL, less than 800 ng/mL, less than 100 ng/mL, less than 150 ng/mL, less than 175 ng/mL, less than 350 ng/mL, less than 990 ng/mL, less than 1980 ng/mL, less than 2000 ng/mL, or less than 3228 ng/mL of the SRA737 compound in the subject after administration.

Clause 114. The method of any of clauses [00212]-[00322], wherein the method results in a plasma Cₘₐₓ between 500 and 3200 ng/mL of the SRA737 compound in the subject after administration.

Clause 115. The method of any of clauses [00212]-[00322], wherein the method results in a plasma Cₘₐₓ between 500 and 2400 ng/mL of the SRA737 compound in the subject after administration.

Clause 116. The method of any of clauses [00212]-[00322], wherein the method results in a plasma Cₘₐₓ between 500 and 650 ng/mL of the SRA737 compound in the subject after administration.

Clause 117. The method of any of clauses [00212]-[00322], wherein the method results in a plasma Cₘₐₓ between 500 and 550 ng/mL of the SRA737 compound in the subject after administration.

Clause 118. The method of any of clauses [00212]-[00322], wherein the method results in a plasma Cₘₐₓ between 500 and 5500 ng/mL of the SRA737 compound in the subject after administration.

Clause 119. The method of any of clauses [00212]-[00322], wherein the method results in a plasma Cₘₐₓ between 500 and 4000 ng/mL of the SRA737 compound in the subject after administration.

Clause 120. The method of any of clauses [00212]-[00330], wherein the subject has fasted prior to administering the effective amount of the SRA737 compound.

Clause 121. The method of clause [00331], wherein the subject has fasted 30 minutes or more, 1 hour or more, 2 hours or more, 3 hours or more, or 4 hours or more prior to administering the effective amount of the SRA737 compound.

Clause 122. The method of clause [00331], wherein the subject has fasted 2 hours or more prior to administering the effective amount of the SRA737 compound.

Clause 123. The method of any of clauses [00212]-[00333], further comprising the subject fasting following administering the effective amount of the SRA737 compound.

Clause 124. The method of clause [00334], wherein the subject fasts 30 minutes or more, 1 hour or more, 2 hours or more, 3 hours or more, or 4 hours or more following administering the effective amount of the SRA737 compound.

Clause 125. The method of clause [00334], wherein the subject fasts 1 hour or more following administering the effective amount of the SRA737 compound.

### EXAMPLES

Below are examples of specific embodiments for carrying out the present invention. the examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Abbreviations

TABLE 1B
Abbreviations

| | |
|---|---|
| QD | Once a day |
| BID | Twice a day |
| BIW | Twice weekly |
| p.o. | Oral(1y) |
| i.p. | Intraperitoneal (1y) |
| RT | Room Temperature |
| BW | Body weight |
| BWL | Body weight loss |
| FFPE | Formalin Fixed Paraffin Embedded |
| SD | Standard deviation |
| SEM | Standard error of mean |
| AE | Adverse event |
| MTD | Maximum tolerated dose |
| HNSTD | Highest non-severely toxic dose |
| NOAEL | No-observed-adverse-effect-level |

### Example 1: Summary of Nonclinical Pharmacology

SRA737 was previously found to be a potent and selective inhibitor of Chk1 with limited off-target activity against other kinases, for example, as described in more detail in Walton et al. (Oncotarget. 2016 January 19; 7(3): 2329-2342), herein incorporated by reference for all it teaches. In vitro, SRA737 potently inhibited genotoxic chemotherapy-induced Chk1 autophosphorylation and prevented downstream signal transduction (data not shown). This Chk1 inhibition produced the expected dose-dependent inhibition of genotoxicity-induced checkpoint arrest and a SRA737 dose-dependent potentiation of the cytotoxicity of genotoxic chemotherapeutic agents and targeted agents.

A program of in vivo efficacy studies was performed to assess the activity of SRA737 in combination with genotoxic chemotherapy and targeted agents, and as monotherapy.

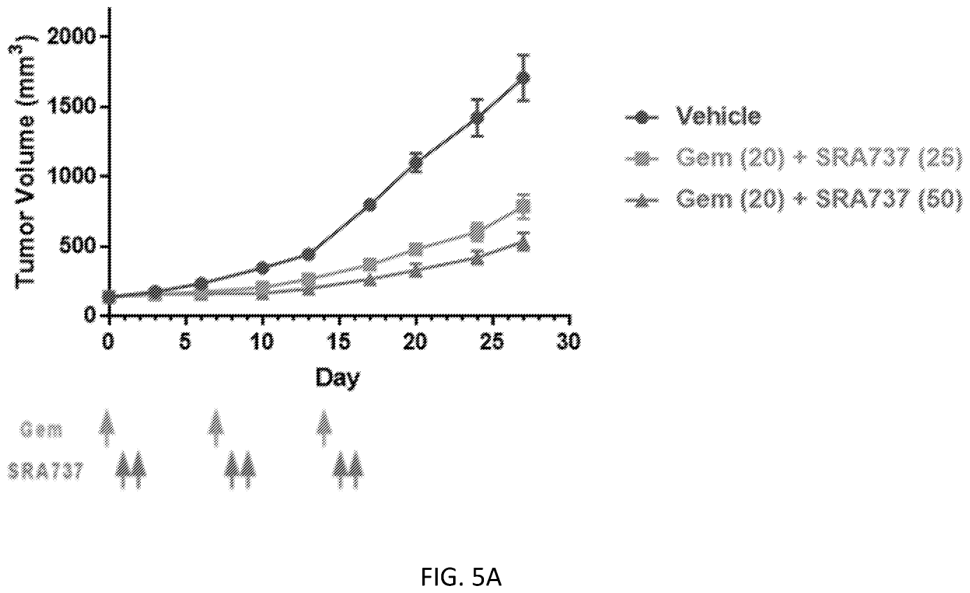

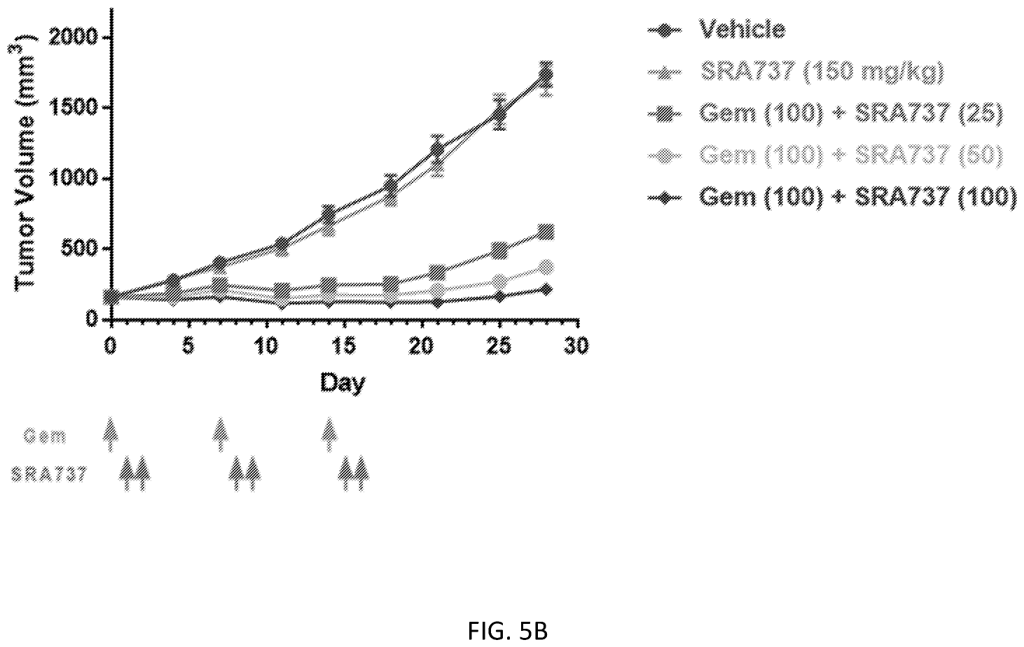

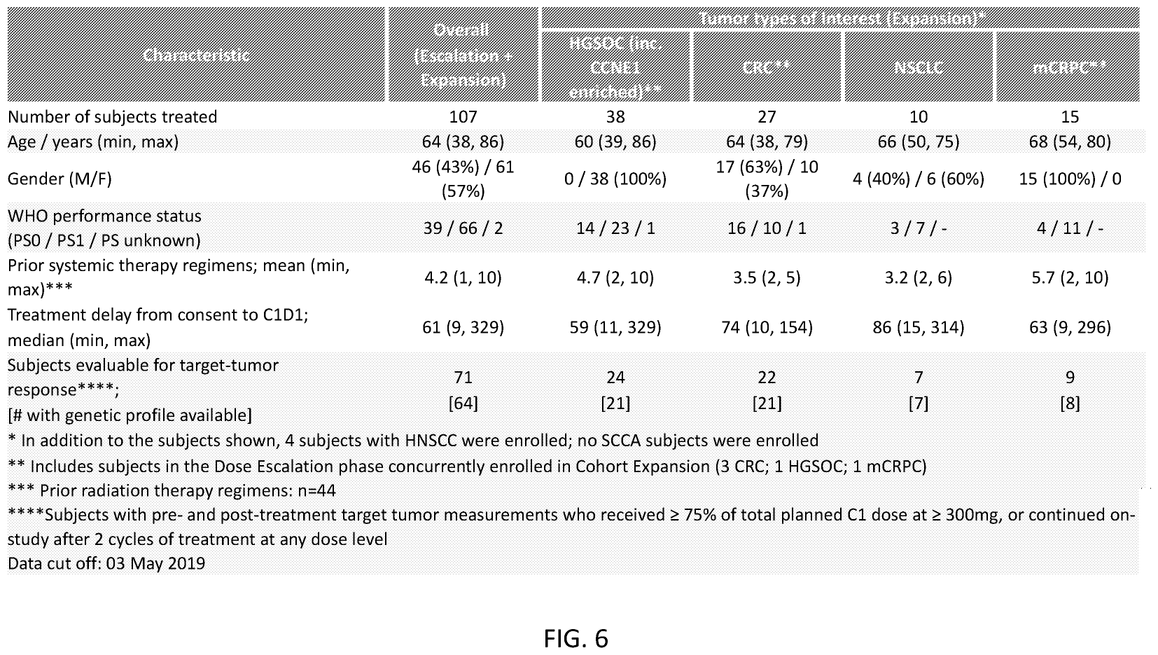

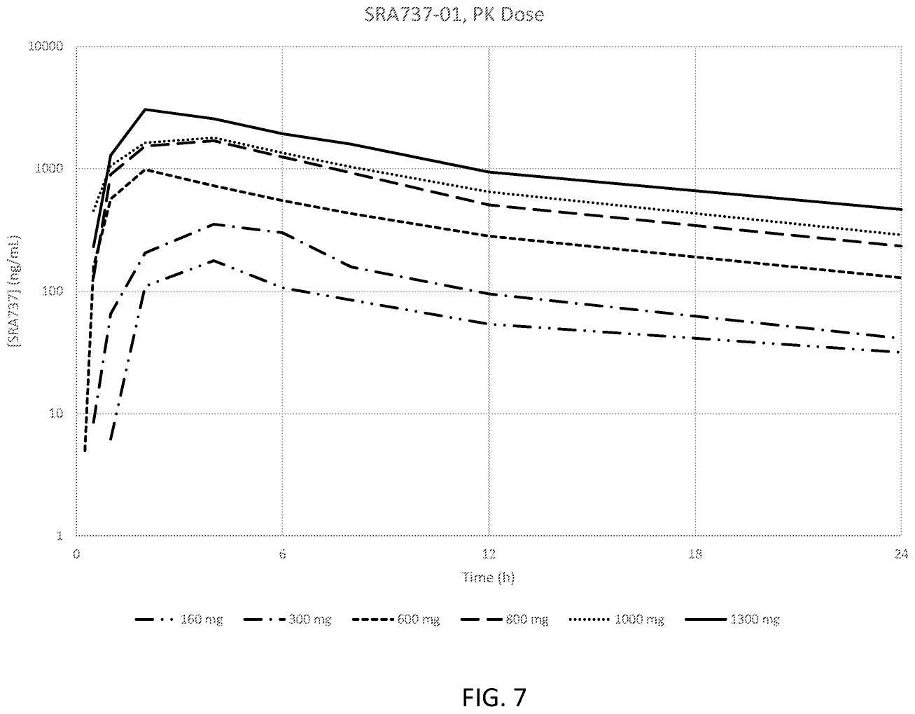

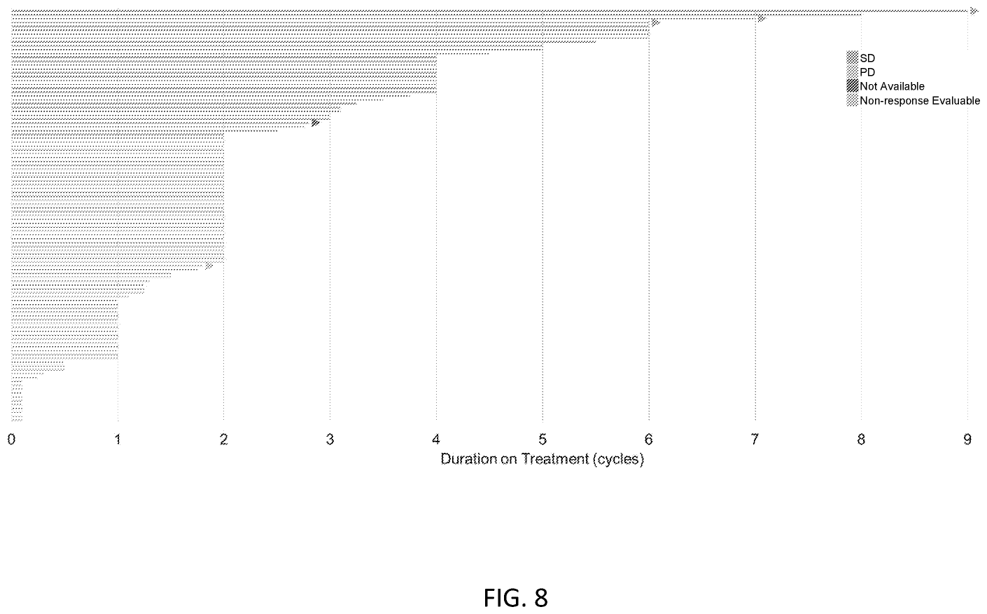

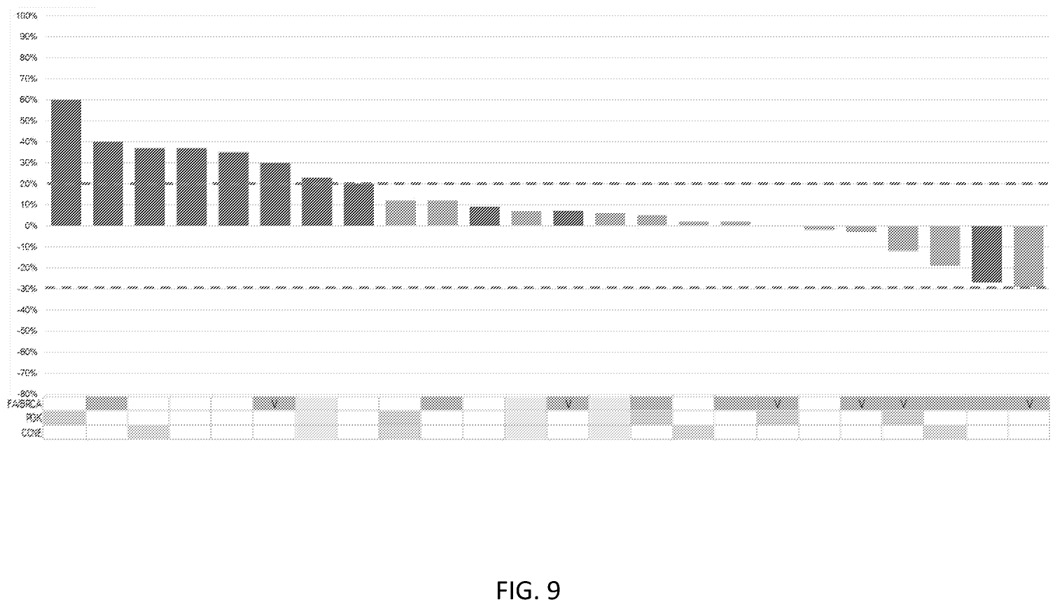

Significant, dose-dependent antitumor activity of SRA737 in combination with standard-dose gemcitabine was noted in multiple cancer xenograft models including HT29 human colon cancer, SJSA-1 human osteosarcoma, SW620 mouse colon cancer, Calu6 human (NSCLC), KPC-1 pancreatic cancer and patient-derived bladder carcinoma (data not shown). Synergy was also observed with low-dose gemcitabine in the HT29 (FIG. 5), OVCAR3 and SJSA-1 CDX models as well as in a PDX model of TNBC; with gemcitabine and carboplatin in the Calu6 model, and with irinotecan in the HT29 model (data not shown). Significant antitumor activity was also observed in three syngeneic mouse models (mTmG, MC38 and Pan02) in combination with a PD1/PD-L1 inhibitor (data not shown). Significant antitumor activity of SRA737 presented as a single agent was observed in several HGSOC PDX models harboring CCNE1 amplifications accompanied by TP53 mutations (data not shown). One of these models also carried a MYCN amplification. A fourth HGSOC PDX model with partial resistance to PARPi was also sensitive to high dose SRA737 monotherapy. SRA737 also demonstrated single agent efficacy in the OVCAR3 model of HGSOC, Eμ-Myc model of B-cell lymphoma; MOLM-13 model of AML; TH-MYC model of neuroblastoma; MDA-MB-231 model of TNBC and in two syngeneic models of renal and lung cancers (Renca and LL/2, respectively) (data not shown).

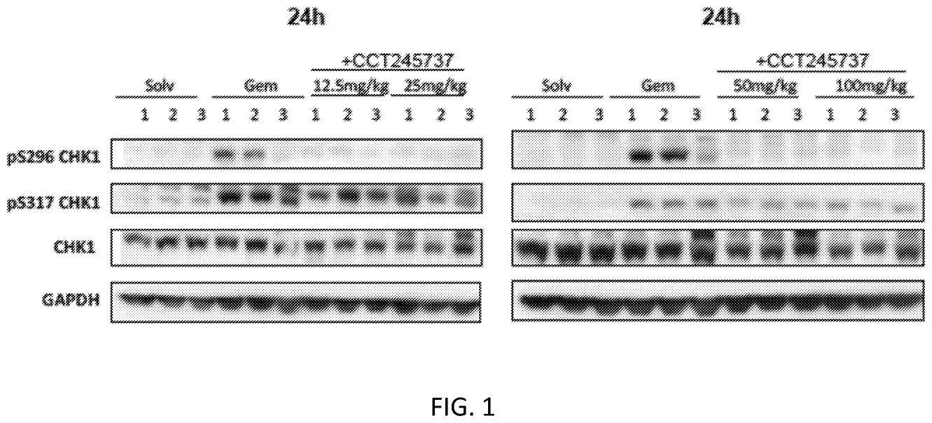

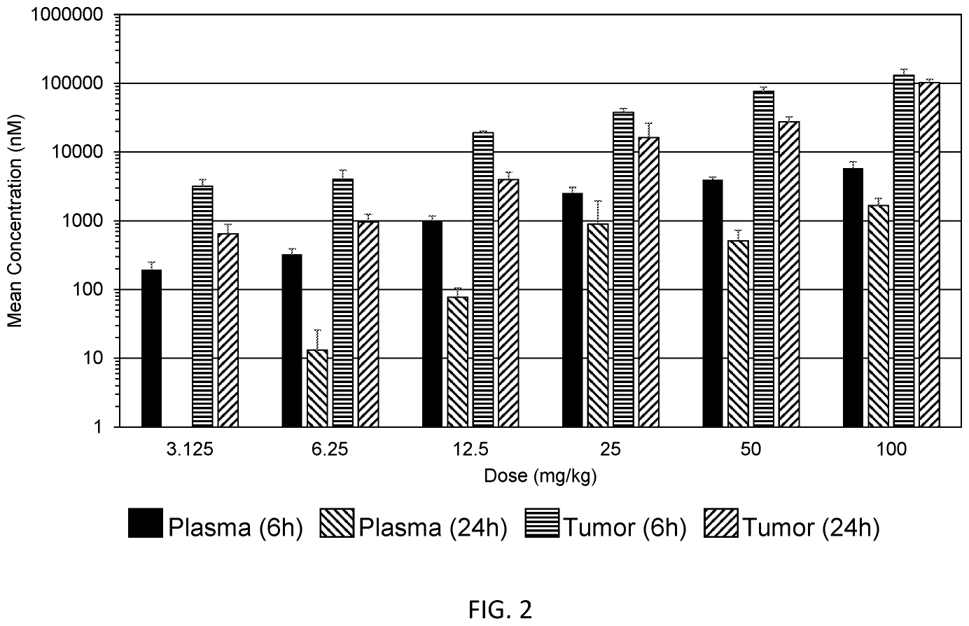

The effect of SRA737 on Gemcitabine induced CHK1 S296 autophosphorylation was assessed as described in Walton et al. (Oncotarget. 2016 January 19; 7(3): 2329-2342), herein incorporated by reference for all it teaches. Briefly, mice bearing HT29 tumor xenografts were administered (i) vehicle control or (ii) gemcitabine (100 mg/kg in saline, IV) or (iii) a combination of SRA737 (12.5, 25, 50 or 100 mg/kg in DTPW, oral) and gemcitabine (100 mg/kg) with SRA737 administered 24 hours following gemcitabine administration (n=3 per time point per treatment). Inhibition of pS296 Chk1 was observed at SRA737 doses greater than or equal to 12.5 mg/kg (FIG. 1), which corresponded to a minimum (total) plasma concentration of approximately 100 nM (actual value 78±27 nM, ˜40 ng/mL) at 24 hours (FIG. 2 and Table 2). Exposure in the tumor was greater than 10-fold higher than in plasma. The circulating plasma concentration at this 24-hour timepoint corresponded to a free drug concentration of approximately 6 nM (2 ng/mL) based on plasma protein binding in mice of 94%.

The PK/PD data showed that relatively low, plasma concentrations of SRA737 sustained above an effective concentration (e.g., SRA737 exceeding a 100 nM plasma concentration for 24 hours) elicited significant antitumor activity in mice and provides a PK/PD benchmark for application in a clinical setting.

TABLE 2
SRA737 plasma concentrations and tumor concentrations in a HT29 xenograft model

| Dose | | | | | | | | |
|---|---|---|---|---|---|---|---|---|
| (mg/kg) | Plasma (6 h) | | Plasma (24 h) | | Tumor (6 h) | | Tumor (24 h) | |
| 3.125 | 193.9256 | 55.89237 | 0 | 0 | 3183.276 | 787.7809 | 645.7623 | 239.2542 |
| 6.25 | 323.3166 | 64.99931 | 13.10728 | 12.62854 | 4011.501 | 1421.794 | 965.7797 | 283.3167 |
| 12.5 | 1007 | 165.5266 | 77.7 | 26.58816 | 19009.57 | 1066.455 | 3974.933 | 1099.057 |
| 25 | 2517.067 | 532.9315 | 893.4667 | 1048.18 | 37516 | 5539.576 | 16289.53 | 9893.223 |
| 50 | 3957.5 | 339.5922 | 512.1 | 215.3084 | 76331.2 | 11510.94 | 27483.73 | 4915.968 |
| 100 | 5837.767 | 1365.599 | 1674.5 | 431.4472 | 131506.7 | 28331.53 | 101764.1 | 11743.44 |

SRA737+LDG is a novel drug combination, where non-cytotoxic low dose gemcitabine (LDG) acts as a potent extrinsic inducer of replication stress that potentiates SRA737's anti-tumor activity. Preclinical models have demonstrated that only subtherapeutic levels of gemcitabine are needed to potentiate SRA737's anti-tumor effect.

### Example 2: Summary of Pharmacokinetic (PK) Studies

Several studies have been conducted to evaluate the PK properties of SRA737, such as the absorption (in vitro permeability assays and in vivo PK following IV and oral administration), distribution (in vivo tissue distribution and in vitro plasma protein binding) and metabolism (in vitro hepatocyte and CYP inhibition and induction studies) of SRA737.

The PK of SRA737 have been determined in the mouse, rat, dog and monkey following oral and IV administration (Table 3). Very favorable absolute oral bioavailability (% F) was noted, particularly in the mouse (105%) and monkey (90-104%), consistent with the moderate metabolism and favorable permeability noted in in vitro models. An acceptable terminal elimination t_{1/2} was also observed in each species. In addition, the effect of prandial state on the PK of the SRA737 clinical drug product capsule presentation was evaluated in dogs. there was no significant effect of prandial state on oral bioavailability (Error! Reference source not found.4). The plasma protein binding of SRA737 at 1 and 10 μM was examined in mouse, minipig, monkey and human plasma using ultracentrifugation and in dog plasma (10 μM) using rapid equilibrium dialysis. Moderate plasma protein binding was observed in humans (˜87%) and the non-rodent toxicology species (˜80% and 87% for the minipig and monkey, respectively), whereas high plasma protein binding (˜94%) was observed in the mouse (Table 5).

TABLE 3
Summary of PK Parameters of SRA737 After a Single Dose

| Species | Dose | Cₘₐₓ | Tₘₐₓ | AUCₗₐₛₜ | t_{1/2} | Fₒᵣₐₗ |
|---|---|---|---|---|---|---|
| and route | (mg/kg) | (μg/mL) | (h) | (μg · h/mL) | (h) | (%) |
| Mouse IV | 10 | — | — | 3.77 | 2.9 | — |
| Mouse oral | 10 | 0.61 | 1 | 3.94 | 2.8 | 105 |
| Rat IV | 5 | — | — | 1.38 | 1.0 | — |
| Rat oral | 10 | 0.19 | 2 | 1.17 | 4.6 | 42 |
| Dog IV | 1 | — | — | 0.20 | 2.8 | — |
| Dog oral | 5 | 0.13 | 1-3 | 0.85 | 3.5 | 86 |
| Monkey IV | 1 | — | — | 1.52 | 5.3 | — |
| Monkey oral | 2 | 0.50 | 1-2 | 2.73 | 5.0 | 90 |
| Monkey oral | 10 | 2.64 | 2-4 | 15.84 | 5.1 | 104 |

TABLE 4
Summary of PK Parameters of SRA737 Drug Product After
a Single Dose in Fasted and Non-Fasted Dogs

| Route/Prandial | | Cₘₐₓ | Tₘₐₓ | AUCₗₐₛₜ | t_{1/2} | Fₒᵣₐₗ |
|---|---|---|---|---|---|---|
| State | Dose | (μg/mL) | (h) | (μg · h/mL) | (h) | (%) |
| IV (Fasted^{a}) | 1 mg/kg | — | — | 0.587 | 2.2 | — |
| IV (Non-fasted^{b}) | 1 mg/kg | — | — | 0.518 | 1.5 | — |
| Oral (Fasted^{a}) | 100 mg/dog | 0.75 | 1.5 | 4.29 | 2.6 | 79 |
| Oral (Non-fasted^{b}) | 100 mg/dog | 0.64 | 2.0 | 3.28 | 2.2 | 65 |

^{a}Fasted: animals fasted overnight and only fed at 4 h postdose
^{b}Non-fasted animals: fastedovernight and fed 1 h predose

TABLE 5
Summary of the Percentage Plasma Protein Binding and
Percentage Free SRA737 in Mouse, Minipig,
Human, Dog and Monkey

| | Percentage Bound | | Percentage Free | |
|---|---|---|---|---|
| Species | 1 μM | 10 μM | 1 μM | 10 μM |
| Mouse | 94.5 | 93.4 | 5.5 | 6.6 |
| Minipig | 85.2 | 75.5 | 14.9 | 24.5 |
| Human | 87.7 | 87.2 | 12.4 | 12.8 |
| Dog | N/A | 68.8 | N/A | 31.2 |
| Monkey | 88.8 | 85.8 | 11.2 | 14.2 |

The membrane permeability of SRA737 was assessed in the parallel artificial membrane permeability assay (PAMPA) and Caco-2 assays. Permeability in the PAMPA assay was classified as low. At 10 μM permeability in the Caco-2 assay was 20.7±9.1×10⁻⁶ cm/s with an efflux ratio (A>B/B>A) of 0.8, which indicated that SRA737 has a relatively high passive permeability and low efflux potential.

The formation of SRA737-related metabolites was determined in cryopreserved hepatocytes from human, mouse, rat, dog, minipig and monkey samples after incubation with SRA737 at a nominal concentration of 10 μM for up to 4 hours. The rank order of stability from most stable to least stable for the species was rat≈mouse>monkey˜human>>dog>>minipig. In the human hepatocyte preparation, approximately 67% of the parent remained after 4 hours of incubation compared to 75% and 7% in the rat and minipig preparations, respectively. Eight human SRA737 metabolites were observed. All SRA737 metabolites formed by human hepatocytes were also formed by monkey hepatocytes. Six metabolites were present at equal or greater abundance in the monkey. No human-specific metabolites were observed, but two of the human metabolites were not formed in any other species at equal or greater abundance.

The excretion of SRA737 has been studied in mice and rats administered SRA737 at either 5 mg/kg IV or 10 mg/kg orally. Urine and feces were collected for a 24-hour period after dosing. In mice, renal excretion of intact SRA737 was consistently low (less than 10% of dose) after both oral and IV administration. Following IV administration of SRA737, less than 8% of the dose was recovered as intact drug in the feces while after oral administration this figure was less than 3%. Excretion of intact SRA737 was lower in the rat than in the mouse with <1% of dose excreted renally over 24 hours and less than 1.5% of dose excreted into the feces over 24 hours.

No significant inhibition of major CYP enzymes (1A2, 2A6, 2C9, 2C19, 2D6 and 3A4) was observed at the highest concentration of SRA737 investigated (IC₅₀>10-50 μM), suggesting that the compound is unlikely to mediate significant metabolic drug-drug interactions. Minimal, concentration dependent induction of CYP1A2 (<10% positive control, omeprazole) was observed in vitro suggesting SRA737 in some cases minimally affected the metabolism of concomitantly administered drugs that are predominately metabolized by CYP1A2.

Taken together, the permeability, metabolic stability, and demonstrable oral bioavailability observed in preclinical species is suggestive of favorable oral absorption in humans.

### Example 3: Summary of Toxicology Findings

The toxicity of SRA737 was assessed in Good Laboratory Practice (GLP) 28-day repeat oral dose studies in the mouse, minipig and monkey and in a three-cycle combination study with gemcitabine and cisplatin in the mouse.

Toxicokinetic data for SRA737 administered as a monotherapy are summarized in Error! Reference source not found.6. The pattern of toxicology findings observed in the pivotal studies in mouse, minipig and monkey were broadly similar and consistent with SRA737's mechanism of action although in general, the monkey appeared to be the least sensitive toxicological species. Data from studies in the monkey suggest higher exposures would likely be tolerated in humans than would be predicted from mouse and minipig data. Based on similarities between monkey and human data for plasma protein binding, stability in hepatocytes, and other ADMET data, the monkey has been confirmed as the most suitable nonclinical model for the determination of potential human toxicity.

Dose-dependent toxicological findings related to bone marrow toxicity, including variously decreased red and white cell parameters with increased medullary or extramedullary hematopoiesis and atrophy of lymphatic organs including the thymus was noted in the mouse and minipig pivotal studies. These findings were reversible on cessation of drug administration. Toxicological findings in the GI tract were also observed in the minipig and in early mouse and monkey studies and changes in reproductive organs, particularly the testes, were also observed in the minipig and mouse, but not monkey. these latter changes were not reversible in the mouse; however, the relevance of these findings in sexually immature animals to adult cancer patients appears limited.

The MTD was 75 mg/kg/day (225 mg/m²/day) in the mouse and the HNSTD was 10 mg/kg/day (350 mg/m²/day) in the minipig. An absence of toxicological findings was noted in the pivotal monkey toxicity study, thus the NOAEL of 20 mg/kg/day (240 mg/m²/day) was the highest dose tested.

Findings from a triple combination study in mice (SRA737 administered in combination with IV gemcitabine and cisplatin on an intermittent schedule over 18 days) mirrored those noted in the monotherapy toxicity study in this species, although reversible intestinal epithelial degeneration was also noted in the high dose triplet combination group. Only the reversible marrow toxicity, consequent splenomegaly, and the high-dose intestinal observations were deemed to have been exacerbated by administration of SRA737 over those observed following administration of the cisplatin/gemcitabine control group alone. Clinical combination of SRA737 and gemcitabine in some cases therefore elicited exacerbated hematological and GI toxicities.

TABLE 6
Summary of the Toxicokinetic Data on SRA737
Administered as a Monotherapy
(Daily Dosing of SRA737 for 28 Days)

| | Dose | Mouse | | Minipig | | Monkey | |
|---|---|---|---|---|---|---|---|
| Parameter | (mg/kg/day) | Male | Female | Male | Female | Male | Female |
| Cₘₐₓ | 2 | — | — | — | — | 0.365 | 0.519 |
| (μg/mL) | 10 | 0.903 | 0.918 | 0.622* | 0.515* | 2.07 | 1.87 |
| | 20 | — | — | — | — | 3.17* | 2.32* |
| | 40 | 3.25 | 2.35 | 2.07 | 2.83 | — | — |
| | 75 | 5.45* | 4.03* | | 5.34 | — | — |
| AUC₀₋ₜ | 2 | — | — | — | — | 3.24 | 2.58 |
| (μg · h/mL) | 10 | 4.41 | 3.61 | 6.48* | 6.56* | 17.9 | 12.9 |
| | 20 | — | — | — | — | 20.7* | 24.2* |
| | 40 | 34.3 | 24.8 | 22.21 | 45.48 | — | — |
| | 75 | 68.6* | 45.5* | — | 97.02 | — | — |
| T_{1/2} | 2 | — | — | — | — | 6.73 | 8.74 |
| (hours) | 10 | — | 5.18 | 9.25* | 13.6* | 8.34 | 6.58 |
| | 20 | — | — | — | — | 6.84* | 6.00* |
| | 40 | 6.22 | 5.09 | 7.66 | 14.3 | — | — |
| | 75 | 8.36* | 6.96* | — | 31.5 | — | — |

*data obtained at the MTD, HNSTD and NOAEL for mouse, minipig and monkey, respectively

### Example 4: Phase 1 Clinical Study to Establish the Maximum Tolerated Dose and Blood Plasma Concentration in a SRA737 Monotherapy

A Phase 1 clinical trial was conducted in ‘all comers,’ i.e. no genetic selection was performed, to establish safety, tolerability and pharmacokinetics (“Dose Escalation Phase”). Cohorts consisting initially of a single subject received escalating doses of SRA737, starting in Cohort 1 with 20 mg/day administered orally on a continuous daily dosing schedule in 28 day cycles. the dose was escalated until the maximum tolerated dose (MTD) was identified.

In the Dose Escalation Phase, 18 subjects received SRA737 in 9 dose level cohorts, from 20 to 1300 mg QD; median treatment duration 62.5 days (range 1 to 226). Dose level cohorts of up to 1000 mg of SRA737 were completed without any dose-limiting toxicities (DLTs). Two of 3 subjects experienced DLTs at the 1300 mg once daily dose, each being an inability to receive 75% of the planned SRA737 dose due to GI intolerability, with the individual GI effects being low grade. Hence, 1300 mg exceeded the maximum tolerated dose with the once daily dosing regimen. A cohort receiving 500 mg twice daily was added to determine if a twice daily dosing schedule can improve GI tolerability, given the half-life of SRA737 is approximately 10 hours. One of 6 subjects experienced DLTs in the 500 mg twice daily cohort; this was an inability to receive 75% of the planned SRA737 dose due to grade 4 thrombocytopenia with grade 3 neutropenia and anemia. Based on overall tolerability and GI events (nausea, vomiting, and diarrhea), subjects were also enrolled at a dose level of 800 mg once daily and was overall better tolerated than 1000 mg (subjects required fewer dose reductions, experienced fewer severe (G3/4) AEs and significantly less fatigue AEs. The maximum tolerated dose (MTD) was established at 1000 mg QD or 500 mg BID.

Pharmacokinetic parameters for the monotherapy cohorts were monitored and are summarized in Table 7. The Cₘₐₓ and AUC₀₋₂₄ at 1000 mg QD were 2391 ng/mL and 26795 ng·h/mL respectively. Cₘᵢₙ was calculated at 1000 mg QD (411 ng/mL) and exceeded that determined in preclinical models to be effective. Doses ≥300 mg QD also exceeded the preclinical models to be effective.

TABLE 7
SRA737 Monotherapy Mean (±SD) Pharmacokinetic Parameters

| SRA737 | Day-7^{a} | | | | Day 15/22^{b} | | | |
|---|---|---|---|---|---|---|---|---|
| Dose | Tₘₐₓ | Cₘₐₓ | AUC₀₋₂₄ | T_{1/2} | Tₘₐₓ | Cₘₐₓ | AUC₀₋₂₄ | T_{1/2} |
| (mg) | (h) | (ng/mL) | (ng · h/mL) | (h) | (h) | (ng/mL) | (ng · h/mL) | (h) |
| 20 | 1.0 | 43.0 | 130 | — | 4.0 | 29.0 | 342 | 17.6 |
| | (—) | (—) | (—) | | (—) | (—) | (—) | (—) |
| 40 | 1.0 | 95.0 | 809 | 9.85 | 2.0 | 75.9 | 1,006 | 16.8 |
| | (—) | (—) | (—) | (—) | (—) | (—) | (—) | (—) |
| 80 | 4.0 | 68.9 | 691 | 9.27 | 2.0 | 81.4 | 1,064 | 15.6 |
| | (—) | (—) | (—) | (—) | (—) | (—) | (—) | (—) |
| 160 | 4.0 | 178 | 1,618 | 13.8 | 2.0 | 217 | 2,515 | 19.8 |
| | (—) | (—) | (—) | (—) | (—) | (—) | (—) | (—) |
| 300 | 4.0 | 353 | 3,152 | 8.59 | 6.0 | 145 | 2,362 | 15.4 |
| | (—) | (—) | (—) | (—) | (—) | (—) | (—) | (—) |
| 600 | 1.8 | 998 | 8,870 | 12.9 | 2.9 | 1,381 | 12,850 | 10.4 |
| | (0.5) | (337) | (3,170) | (1.35) | (1.1) | (819) | (5,943) | (1.92) |
| 800 | 3.3 | 1,980 | 18,856 | 10.3 | 2.5 | 2,573 | 25,423 | 10.0 |
| | (1.2) | (633) | (6,211) | (1.34) | (0.9) | (690) | (8,693) | (4.71) |
| 1000 | 3.3 | 2,099 | 20,283 | 11.0 | 3.1 | 2,331 | 25,991 | 9.38 |
| | (1.7) | (814) | (7,744) | (1.72) | (1.8) | (687) | (8,549) | (2.20) |
| 1300 | 2.7 | 3,228 | 29,796 | 11.8 | — | — | — | — |
| | (1.2) | (1,320) | (11,233) | (3.11) | | | | |

^{a}20, 40, 80, 160, 300 mg - n = ; 600 mg - n = 4; 800 mg - n = 25; 1000 mg - n = 43-44; and 1300 mg - n = 3
^{b}20, 40, 80, 160, 300 mg - n = 1; 600 mg - n = 7; 800 mg - n = 12; 1000 mg - n = 17

### Example 5: Phase 1 Clinical Study to Establish the Maximum Tolerated Dose and Blood Plasma Concentration in a SRA737 Combination Therapy

A Phase 1 clinical trial was conducted in ‘all comers,’ i.e. no genetic selection was performed, to establish safety, tolerability and pharmacokinetics for SRA737 administered in combination with gemcitabine (“Dose Escalation Phase”). Cohorts consisting initially of a single subject received escalating doses of SRA737, starting in Cohort 1 with 40 mg/day administered orally on days 2, 3, 9, 10, 16, and 17 of each 28-day cycle. Cohorts also received various doses of gemcitabine, starting in Cohort 1 with 300 mg/m²/day administered IV over 30 minutes on days 1, 8, and 15 of each 28-day cycle.

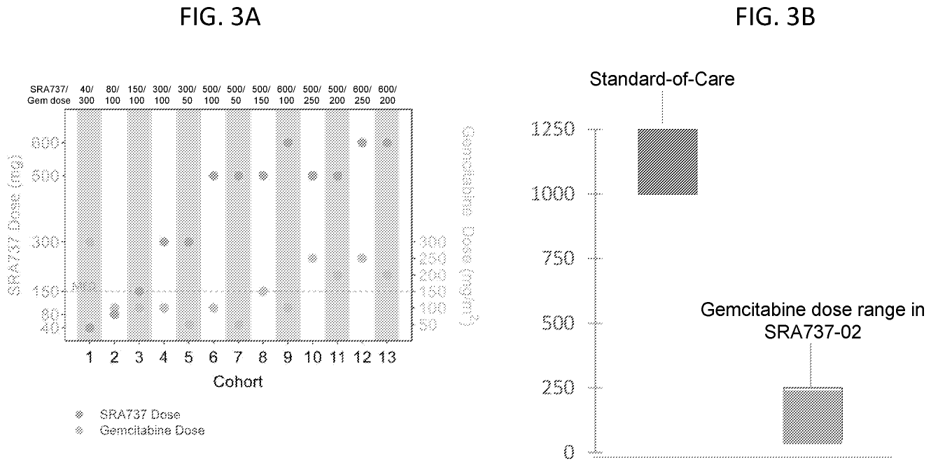

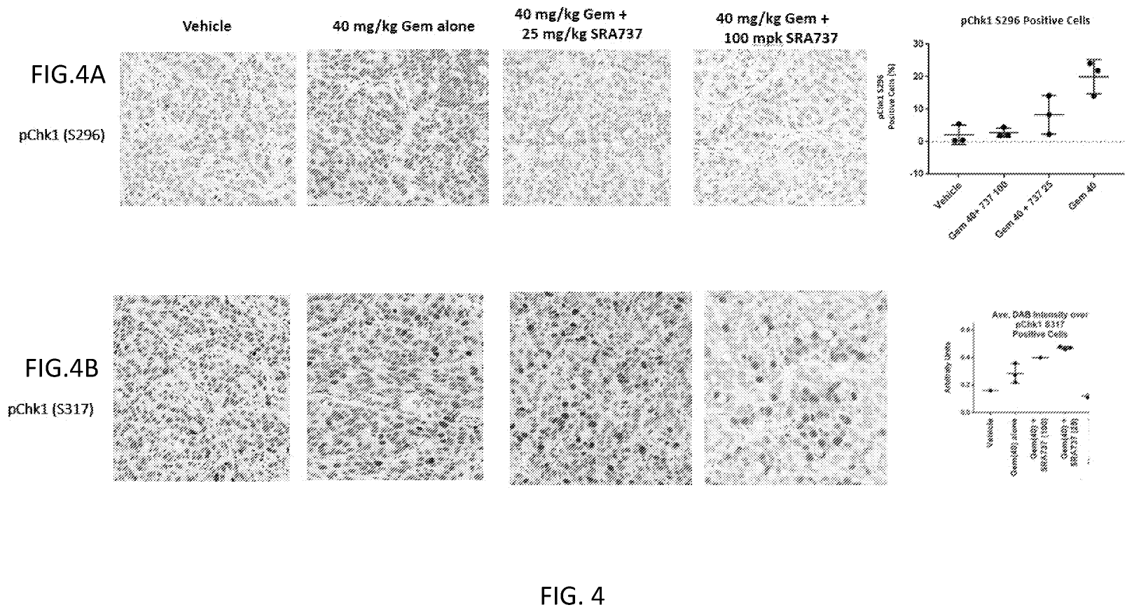

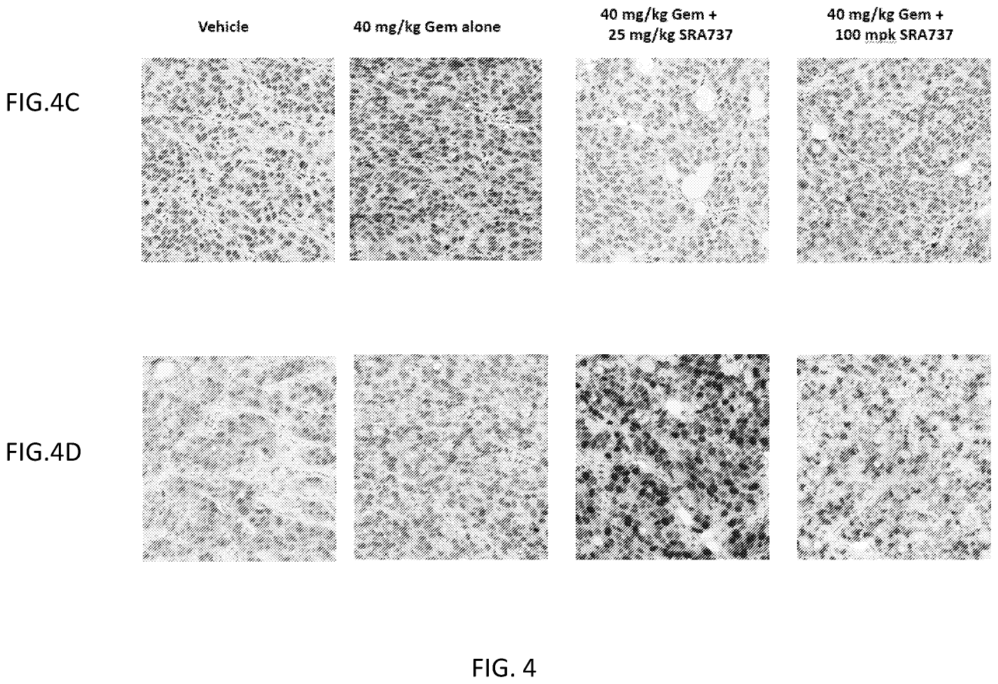

FIG. 3A presents a summary of dosing amounts for the cohorts tested. In the Dose Escalation Phase, a total of 55 subjects received SRA737 in 13 dose escalation cohorts at doses of 40 to 600 mg SRA737 combined with LDG doses of 50 to 300 mg/m². No protocol-defined dose limiting toxicities (DLTs) have been observed.

Pharmacokinetic parameters for the monotherapy cohorts were monitored and are summarized in Table 8. The pharmacokinetic profile of SRA737 revealed AUC₀₋₂₄ and Cₘₐₓ of 3550 ng-h/mL and 548 ng/mL at 150 mg SRA737. At this dose, the Cₘᵢₙ (52 ng/mL) exceeded that determined in preclinical models to be effective.

TABLE 8
SRA737 Combination Therapy Mean (±SD) Pharmacokinetic Parameters

| SRA737 | Day-7^{a} | | | | Day 10^{b} | | | |
|---|---|---|---|---|---|---|---|---|
| Dose | Tₘₐₓ | Cₘₐₓ | AUC₀₋₁₂ | AUC₀₋₂₄ | T_{1/2} | Tₘₐₓ | Cₘₐₓ | AUC₀₋₁₂ |
| (mg) | (h) | (ng/mL) | (ng · h/mL) | (ng · h/mL) | (h) | (h) | (ng/mL) | (ng · h/mL) |
| 20 | 1.9 | 31.3 | 145 | — | 4.09 | — | — | — |
| | (0.9) | (17.3) | (67.9) | | (1.31) | | | |
| 40 | 2.0 | 106 | 486 | 637 | 11.6 | 1.8 | 117 | 543 |
| | (0.0) | (35.9) | (116) | (140) | (2.95) | (0.5) | (32.0) | (160) |
| 80 | 2.0 | 146 | 641 | 825 | 11.3 | 2.0 | 113 | 570 |
| | (0.0) | (28.6) | (143) | (176) | (0.75) | (0.0) | (26.7) | (100) |
| 150 | 1.8 | 548 | 2,630 | 3.550 | 12.7 | 1.5 | 331 | 1,677 |
| | (0.5) | (63.9) | (944) | (1,516) | (1.13) | (0.9) | (307) | (1,439) |
| 300 | 2.4 | 995 | 4,534 | 5,907 | 11.8 | 1.7 | 1,079 | 5,140 |
| | (1.6) | (449) | (1,593) | (1,882) | (0.99) | (1.1) | (564) | (1,606) |
| 500 | 2.4 | 1,433 | 8,318 | 11,417 | 11.8 | 2.4 | 1,723 | 10,191 |
| | (1.4) | (729) | (4,603) | (6,678) | (2.01) | (2.6) | (865) | (5,772) |
| 600 | 1.8 | 2,208 | 11,805 | 15,072 | 11.1 | 2.5 | 2,109 | 11,860 |
| | (0.5) | (359) | (2,477) | (3,041) | (1.31) | (1.0) | (536) | (3,241) |

^{a}20 mg - n = 8-10; 40 mg - n = 6; 80 mg - n = 3; 150 mg - n = 4; 300 mg - n = 7; 500 mg - n = 29; and 600 mg - n = 4
^{b}40 mg - n = 4; 80, 150 mg - n = 3; 300 mg - n = 7; 500 mg - n = 19-22; and 600 mg - n = 4

Additional cohorts are monitored escalating the dose of SRA737 until the maximum tolerated dose (MTD) is identified and to optimize combination dosing with gemcitabine. All enrolled subjects who receive at least 1 dose of SRA737 and provide at least 1 evaluable PK concentration or have evaluable data for each specific PDn assessment are evaluable for PK and PDn, respectively. Serious adverse events (SAEs) are collected starting on the date of informed consent. Radiological assessment are performed within 4 weeks from the first dose of SRA737 (or gemcitabine if the SRA737 dose for PK is omitted) and repeated every 6 weeks in Stage 1. In Stage 2, assessments are performed every 8 weeks and in long-term follow-up every 16 weeks. Assessments are performed more frequently, when clinically indicated. Cardiac assessments (echocardiogram [ECHO] and electrocardiogram [ECG]) are be conducted. Optional triplet tumor biopsies in some cases are collected within 28 days prior to receiving the first SRA737 dose. Within 7 days of the first dose of SRA737 (or gemcitabine if the SRA737 dose for PK is omitted), the following assessments are completed: complete physical examination, clinical disease assessment, SAE and concomitant medication, WHO performance status and local laboratory assessment of blood (for hematology, biochemistry, and pregnancy testing). At the single-dose PK run-in on Day −7 to Day −4 visit, concomitant medication, vital signs (including temperature, blood pressure, and pulse), height, weight, body surface area (BSA), and WHO performance status are collected. Blood samples are obtained predose for hematology, biochemistry, pregnancy testing, troponin I or T, as well as for tumor markers and tumor profiling. Adverse events (AEs) are collected starting at the administration of SRA737. Archival tissue is submitted for tumor profiling. PK samples are collected at up to 10 time points over a 48-hour time period on Day −7 to −4 (first dose of SRA737 for PK). The sponsor in some cases reduces the requirement for PK sampling, including modification or elimination of the Day −7 to Day −4 visit once sufficient data to evaluate the single-dose PK of SRA737 have been collected and analyzed. Dosing begins on Day 1 with the following procedures occurring at regular intervals:
Adverse event and concomitant medication: On an ongoing basis
Symptom-directed physical exam (if medically indicated): Day 1 of each cycle
Radiological disease assessment: Every 6 weeks after Day 1 for Stage 1 and every 8 weeks after Day 1 for Stage 2
Clinical disease assessment: Every 6 weeks after Day 1 for Stage 1 and every 4 weeks after Day 1 for Stage 2
Tumor markers (serum or urine) (if applicable): every 6 weeks from Cycle 1 Day 1 for Stage 1 and every 4 weeks from Cycle 1 Day 1 for Stage 2
WHO Performance Status and weight/BSA: Day 1 of each cycle
Vital signs: Days 1 and 8 for Stage 1 and Days 1, 8, and 15 for Stage 2
Local laboratory assessment of blood (for hematology, biochemistry, troponin I or T) and urine (urinalysis)—see Section 7.2.2 for detailed schedule
ECHO: Cycle 2 Day 1
ECG: Day 1 of Cycles 1 and 2, and then predose Day 1 at each third subsequent cycle, and Day 1 of any cycle with intra-subject dose escalation
Pharmacokinetics Samples for PK are taken at the following timepoints in Cycle 1: pre-dose on Day 1, pre-dose on Day 10, up to 6 time points post-dose on Day 10.
For Stage 2 only, additional samples are taken pre-dose on Day 8 and pre-dose on Day 15. Compliance review of subject diary card.

### Example 6: Phase 1/2 Clinical Study to Confirm Efficacy of SRA737 Monotherapy in Select Tumors with Genetic Alterations that Confer Chk1 Sensitivity

An in-human clinical trial is conducted to confirm efficacy of SRA737 monotherapy methods of treatment and patient selection strategies disclosed herein for prospectively-selected genetically-defined subjects with tumor types known to have a high prevalence of genomic alterations expected to sensitize the tumor to Chk1 inhibition (“Cohort Expansion Phase”). The Cohort Expansion Phase consists of 6 indication-specific expansion cohorts of approximately 20 prospectively-selected genetically-defined subjects each. The cohorts are subjects with previously treated metastatic colorectal cancer [CRC], high grade serous ovarian cancer [HGSOC] without CCNE1 gene amplification, HGSOC with CCNE1 gene amplification (or alternative genetic alteration with similar functional effect), metastatic castration-resistant prostate cancer [mCRPC], advanced non-small cell lung cancer [NSCLC], and squamous cell carcinoma of the head and neck [HNSCC], or squamous cell carcinoma of the anus [SCCA]. Subjects are initially administered SRA737 following the dosing regimen established in Example 4. The dosing regimen in some cases changes during the course of the trial.

Subjects have tumor tissue or ctDNA evidence that their tumor harbors a combination of mutations which are expected to confer sensitivity to Chk1 inhibition. Subjects are selected based on prospective, tumor tissue genetic profiling using NGS.

Expansion cohort subjects have tumors that harbor genomic alterations expected to confer sensitivity to Chk1 inhibition in a minimum of two of the following categories (a)-(e):
a. Key tumor suppressor genes regulating G1 cell cycle progression/arrest such as RB1, TP53, etc. For patients with NHSCC or SCCA, positive HPV status is also considered for eligibility.
b. The DDR pathway including ATM, CDK12, BRCA1, and BRCA2. For patients with CRC, mismatch repair (MMR) genetic alterations and/or high microsatellite instability are also considered for eligibility.
c. Genetic indicators of replicative stress such as gain of function/amplification of Chk1 or ATR or other related gene.
d. Oncogenic drivers such as MYC, KRAS, etc.
e. CCNE1 gene amplification (or alternative genetic alteration with similar functional effect) is required for the CCNE1 gene amplification-specific HGSOC cohort.

In some aspects, subjects meet one of the following criteria (a-e):
a. Metastatic CRC
   i. Histologically and/or cytologically confirmed CRC
   ii. In general, have received at least 1 prior regimen for advanced/metastatic disease
b. HGSOC
   i. Histologically confirmed high grade serous ovarian, fallopian tube or primary peritoneal cancer
   ii. Recurrent platinum-intolerant subjects, or those with platinum-resistant disease, defined as radiological evidence of disease progression within 6 months of the last receipt of platinum-based chemotherapy. Patients with platinum refractory disease (as defined by the European Society for Medical oncology [ESMO] Guidelines) are not eligible.
c. Advanced NSCLC
   i. Locally advanced and recurrent or metastatic, histologically confirmed NSCLC
   ii. In general, have received at least 1 prior regimen for advanced/metastatic disease
d. mCPRC
   i. Histologically or cytologically confirmed adenocarcinoma of the prostate that has progressed after androgen deprivation therapy
e. HNSCC or SCCA
   i. Histologically confirmed HNSCC from any primary site, or SCCA
   ii. For HNSCC: locally advanced disease (i.e., persistent or progressive disease following curative-intent radiation, and not a candidate for surgical salvage due to incurability or morbidity), or metastatic disease
   iii. For SCCA: locally advanced disease or metastatic disease for which no curative intent therapy is available.
   iv. Subjects have received at least 1 prior regimen for advanced/metastatic disease.

Subjects, in general, have measurable disease (per Response Evaluation Criteria in Solid Tumors, version 1.1 [RECIST v1.1]) or, for mCRPC, evaluable disease per any of the following: Measurable disease per RECIST v1.1; increasing prostate specific antigen (PS); or circulating tumor cell (CTC) count of 5 or more cells per 7.5 ml of blood.

Enrollment to Expansion Cohorts in some cases occurs in parallel with the Dose Escalation Phase (see Example 5). A subject that qualifies for the Cohort Expansion Phase is enrolled into an Escalation Cohort whenever possible. Any such subject is considered to have enrolled in both phases simultaneously.

Disease is measured according to the RECIST v1.1 criteria for subjects with solid tumors, according to the revised IWG criteria (Cheson 2007) for subjects with NHL, and for subjects with mCRPC, using a composite of any one of the following: A) Measurable disease per RECIST v1.1; B) Increasing PSA; or C) CTC count of 5 or more cells per 7.5 ml of blood.

Baseline evaluations include radiological measurements of lesions appropriate to the nature of the malignancy. In some cases, this includes: CT scan, liver CT scan, abdominal CT scan, MRI, X-ray, bone scan and/or other radiological measurements as clinically indicated or clinical measurements as appropriate (e.g., assessment of palpable lesions or measurement of tumor markers). All areas of disease present are documented (even if specific lesions are not going to be followed for response) and the dimensions of all measurable lesions are recorded clearly on the scan reports. Any non-measurable lesions are stated as being present. For clinical measurements, documentation by color photography including a ruler to estimate the size of the lesion is strongly recommended, as this aids external independent review of responses.

Tumor assessments is repeated every 8 weeks or more frequently, when clinically indicated. Subjects with bone metastases being followed by bone scans are scanned every 8 weeks (±1 week) for the first 6 months and then every 16 weeks (±2 weeks) thereafter. During Long-term Follow-up, assessments for subjects who have not yet progressed and who have not initiated alternative anti-cancer therapy are done every 16 weeks, unless requested more frequently by the sponsor or investigator. All lesions measured at baseline are measured at every subsequent disease assessment, and recorded clearly on the scan reports. All non-measurable lesions noted at baseline are noted on the scan report as present or absent. All subjects, who are removed from the study treatment for reasons other than progressive disease (PD), should be re-evaluated at the time of treatment discontinuation, unless a tumor assessment was performed within the previous four weeks. Subjects are followed for PD until disease progression or withdrawal from trial.

All subjects who have measurable disease, receive at least one cycle of SRA737 and have a baseline plus at least 1 post-baseline assessment of disease are evaluable for response. Subjects who develop clear evidence of PD without a formal disease assessment and those without a formal disease assessment before study withdrawal are considered non-responders. Complete responses and PRs are required to be confirmed by a subsequent assessment at least 4 weeks later. Stable Disease (SD) determination requires that the relevant criteria be met at least once, a minimum of 6 weeks after the initial dose of SRA737 is given.

Should rapid tumor progression occur before the completion of 4 weeks of treatment, the subject is classified as having early progression.

Tumor response should be classified as “not evaluable” (NE), only when it is not possible to classify it under another response category, for example, when baseline and/or follow-up assessment is not performed or not performed appropriately.

Response criteria are defined below:
a. Complete Response (CR): Disappearance of all target lesions. Any pathological lymph nodes (whether target or non-target), in general, have reduction in short axis to <10 mm.
b. Partial Response (PR): At least a 30% decrease in the sum of diameters of target lesions, taking as reference the baseline sum diameters.
c. Progressive Disease (PD): At least a 20% increase in the sum of diameters of target lesions, taking as reference the smallest sum on study (this includes the baseline sum if that is the smallest on study). In addition to the relative increase of 20%, the sum, in general, also demonstrates an absolute increase of at least 5 mm. (Note: the appearance of one or more new lesions is also considered progression).
d. Stable Disease (SD): Neither sufficient shrinkage to qualify for PR nor sufficient increase to qualify for PD, taking as reference the smallest sum diameters while on study.

### Results

A Dose Escalation phase employed an accelerated titration design starting at 20 mg SRA737, administered QD orally in 28-day cycles. Incremental dose escalations in single-subject cohorts were followed by a rolling-6 design once SRA737-related ≥Grade 2 toxicity was observed during Cycle 1.

The Cohort Expansion phase was contemporaneously initiated when circulating plasma concentrations of SRA737 exceeded the minimum effective concentration of SRA737 modelled from murine efficacy studies. Thereafter, experience gained in the ongoing Dose Escalation phase informed dose selection for expansion cohorts. The Cohort Expansion phase enrolled subjects with genetically defined tumors that harbored genomic alterations hypothesized to confer sensitivity to Chk1 inhibition, which were prospectively selected by next-generation sequencing (FoundationOne). Subjects with the following tumors were eligible for enrollment: ia) high grade serous ovarian cancer (HGSOC), ib) HGSOC putatively enriched for CCNE1 gene network amplification, ii) colorectal cancer (CRC), iii) metastatic castration-resistant prostate cancer (mCRPC), iv) non-small cell lung cancer (NSCLC), and v) squamous cell carcinomas (head and neck (SCCHN); anus (SCCA)).

This signal-seeking Phase 1/2 study (NCT02797964) was designed to investigate the safety and tolerability of continuous, daily dosing of SRA737, as well as to evaluate preliminary anti-tumor activity in tumors with genetic alterations that may confer increased intrinsic RS and Chk1i sensitivity. Genetic screening was performed to identify and select subjects harboring two or more of these genetic alterations. The study was designed as a broad survey in order to assess the association between various sources of intrinsic RS and SRA737 monotherapy anti-tumor activity in a variety of cancer indications.

Dose limiting toxicities (DLTs) were assessed following a pharmacokinetic (PK) lead-in dose, and throughout Cycle 1 of treatment, and were defined as adverse events highly probably or probably related to SRA737: grade ≥4 neutropenia or thrombocytopenia lasting >7 days, grade ≥3 febrile neutropenia or thrombocytopenia with bleeding, grade ≥3 non-hematologic toxicity, or the inability to receive ≥75% of the planned SRA737 dose in Cycle 1 due to drug-related toxicity.

Preliminary anti-tumor activity was assessed by target tumor response and overall response in accordance with RECIST v1.1.

### Dose and Pharmacokinetics (PK)

In the Dose Escalation phase, 18 subjects received SRA737 across 9 dose level cohorts, ranging from 20 to 1300 mg QD (i.e., 20, 40, 80, 160, 300, 600, 1000 and 1300 mg). Of these subjects, 3 experienced DLTs (inability to receive 75% of the planned dose); 2 at 1300 mg QD due to gastrointestinal intolerability and 1 at 500 mg BID due to thrombocytopenia. The maximum tolerated dose (MTD) was established at 1000 mg QD or 500 mg BID.

The plasma pharmacokinetics of SRA737 were reproducible and generally dose concordant. The Cₘₐₓ and AUC0-24 at the 1000 mg PK dose were 2098 ng/mL and 20270 ng-h/mL, respectively and the Cₘᵢₙ (289 ng/mL) exceeded that determined in preclinical efficacy models to have anti-tumor activity (100 nM; ca 37.9 ng/mL). All doses ≥300 mg QD also exceeded this threshold level.

TABLE 9
Pharmacokinetic data

| | Dose | Tₘₐₓ* | Cₘₐₓ | Cₘᵢₙ | AUC_{0-24 h} | t_{1/2} | CL/F | Vz/F |
|---|---|---|---|---|---|---|---|---|
| PK Dose | (mg) | (h) | (ng/mL) | (ng/mL) | (ng · h/mL) | (h) | (L/h) | (L) |
| | 160 | 4 | 178 | 31.7 | 1,618 | 13.8 | 70.0 | 1,392 |
| | (n = 1) | (—) | (—) | (—) | (—) | (—) | (—) | (—) |
| | 300 | 4 | 353 | 41.2 | 3,152 | 8.6 | 81.9 | 1,015 |
| | (n = 1) | (—) | (—) | (—) | (—) | (—) | (—) | (—) |
| | 600 | 2 | 998 | 129 | 8,870 | 12.9 | 56.9 | 1,090 |
| | (n = 4) | (1-2) | (337) | (49.4) | (3,170) | (1.4) | (20.8) | (516) |
| | 800 | 4 | 1,884 | 234 | 18,537 | 10.5 | 39.1 | 596 |
| | (n = 38) | (1-6) | (606) | (98.2) | (6,056) | (1.8) | (14.9) | (281) |
| | 1000 | 4 | 2,098 | 289 | 20,270 | 11.0 | 44.9 | 704 |
| | (n = 44-45) | (0.5-8) | (805) | (151) | (7,656) | (1.7) | (22.5) | (398) |
| | 1300 | 2 | 3,228 | 466 | 29,796 | 11.8 | 38.6 | 625 |
| | (n = 3) | (2-4) | (1,320) | (234) | (11,233) | (3.1) | (19.7) | (221) |

Enrollment of the Cohort Expansion phase was initiated at 600 mg SRA737. Based on overall tolerability, including particularly common gastrointestinal events (nausea, vomiting, and diarrhea), the recommended dose to be employed in the expansion cohorts was later determined to be 800 mg QD (RP2D).

In the Cohort Expansion phase, of the 512 subjects prospectively identified, 355 were screened for genetic alterations associated with Chk1 sensitivity. Of these subjects 237 (67%) met genetic eligibility criteria, and 94 were enrolled into expansion cohorts across six tumor types.

### Safety

Treatment-emergent adverse events (TEAEs) were reported in 106 (99%) of subjects and 97 subjects (91%) experienced at least one SRA737-related event. The majority of TEAEs were mild to moderate in severity (90% Grade 1/Grade 2). The most common TEAEs were diarrhea (68%), nausea (66%), vomiting (51%) and fatigue (47%).

The most common ≥Grade 3 TEAEs were neutropenia and disease progression (8% each), lymphocyte count decreased (5%), infection and hyponatremia (4% each) and nausea, abdominal pain, abdominal pain upper, fatigue, AST increased, dyspnea, pleural effusion, and rash maculo-papular (3% each). The most common ≥Grade 3 SRA737-related TEAEs were neutropenia (8%) and rash maculo-papular (3%).

Six Grade 5 TEAEs were reported up to 30-days post last treatment; none were considered related to SRA737. Median duration of exposure was 2.3 cycles (range <1 to 9 cycles). No evidence of emergent or cumulative toxicity and/or declining tolerability was observed with up to 9 cycles of SRA737 administered.

TABLE 10
Summary of Treatment-emergent adverse events (TEAEs).
TEAEs regardless of the investigator's assessment
of causality; data cutoff Mar. 23, 2019

| | Occurring in | |
|---|---|---|
| | ≥20% subjects | ≥ Grade 3 n% |
| TEAE | N = 107, n (%) | N = 107, n (%) |
| Subjects with any TEAE | 106 (99.1%) | 73 (68.2%) |
| Diarrhea | 73 (68.2%) | 2 (1.9%) |
| Nausea | 71 (66.4%) | 3 (2.8%) |
| Vomiting | 55 (51.4%) | 1 (0.9%) |
| Fatigue | 50 (46.7%) | 3 (2.8%) |
| decreased appetite | 25 (23.4%) | 0 |
| anemia | 23 (21.5%) | 2 (1.9%) |

### Response

107 subjects have received treatment with SRA737 across both escalation and expansion cohorts. In the Cohort Expansion phase, the largest number of subjects were enrolled in the HGSOC cohort (n=38) with the next largest number in the CRC cohort (n=27).

The mean number of prior treatment regimens across all tumor types was 4.2, consistent with a heavily pretreated study population; the HGSOC cohort had received ˜5 prior lines of therapy.

The median treatment delay from consent to Cycle 1 Day 1 to allow for genetic profiling was ˜2 months (61 days), highlighting the challenge of prospective genetic screening using tumor tissue.

Of the subjects treated, 64 (60%) were considered evaluable for RECIST target tumor response and had an available genetic profile.

A best overall response of SD was seen in 34 (32%) subjects. At the time of the data cut off (3 May 2019), durable stable disease (SD) lasting ≥4 months was recorded in 22 (21%) subjects and was observed in all Cohort Expansion tumor types (except SCCHN; 4 subjects).

Although no subject had a confirmed RECIST partial or complete response, a subset of subjects demonstrated notable target tumor decreases. Anti-tumor activity was observed in subjects with HGSOC, CRC, mCRPC and NSCLC tumors.

HGSOC appeared to be most sensitive tumor to SRA737 monotherapy in this study (maximal target tumor decreases of 27% and 29%; Disease Control Rate (DCR)=54%).

In keeping with the signal seeking objective of this study, tumor responses were further examined with respect to the genetic profiles determined for the tumor types enrolled and treated.

RS-driver genes encompassing functional categories (G1/S, Oncogenes, DNA repair genes) were surveyed to identify gene networks and/or individual genes that enriched for sensitivity to treatment with SRA737.

The number of subjects with tumor alterations in the selected gene networks varied based on i) the occurrence of particular genetic alterations within the indications explored and ii) enrollment metrics.

Although a cohort of subjects was specifically enrolled to putatively enrich for CCNE family mutations, genetic analysis revealed that only 4/24 HGSOC subjects had tumors harboring unambiguously positive CCNE1 amplification, resulting in 3 SD and 1 Progressive Disease (PD). Albeit a very limited dataset, no clear correlation with sensitivity was observed in association with CCNE1 amplification.

Among gene networks with >10 subjects, activating mutations in the RAS network trended with poor DCR (25%) with 15/20 PDs and an overall average tumor % change of +20%, as well as short duration on study (DOS) of 2 cycles. Given the strong negative correlation with activity, subsequent pathway/signal searching analyses was performed with exclusion of RAS mutated tumors.

In contrast to RAS mutations, alterations in the PI3K gene network (PIK3CA, AKT, PTEN) were associated with a 77% DCR.

Similarly, genetic alterations in multiple components of the FA/BRCA gene network were associated with a 71% DCR and DOS of 3.8 cycles.

The majority of subjects with notable responses (tumor reduction and/or >4 months SD) harbored alterations in either or both the PI3K and FA/BRCA gene networks. CCNE gene network alterations (DCR=67%) partially overlapped with FA/BRCA subjects but was mutually exclusive with PI3K network alterations. Many of the subjects demonstrating tumor decrease harbored two genetic alterations in the FA/BRCA gene network, frequently including a secondary mutation in a DDR kinase gene (ATR, PRKDC).

### Summary

In this first-in-human trial of SRA737 monotherapy, the maximum tolerated dose (MTD) was 1000 mg/day, and based on overall tolerability and PK, the recommended monotherapy dose is 800 mg/day. These results highlight the safety and tolerability of SRA737.

The median treatment delay from consent to C1D1 (day 1 of treatment cycle 1) to allow for genetic profiling exceeded 2 months, highlighting the challenge of prospective genetic screening using tumor tissue. In a population of subjects with advanced disease (4+ prior lines), this delay in treatment initiation arguably exacerbates underlying disease progression.

The signal-seeking study surveyed broadly across tumor indications and tumor RS-driver genetics to identify potential SRA737-sensitive settings. Preliminary evidence suggests several intrinsic sources of RS may contribute to or enhance Chk1 dependency across several solid tumor types.

The heavily pre-treated HGSOC cohort (˜5 prior lines) demonstrated directionally favorable disease control (DCR=54%) with notable maximal tumor reductions of 29% and 27%. No clear trend toward enhanced sensitivity was noted with CCNE1 gene amplification; the small subset of subjects enrolled with unambiguous CCNE1 amplifications renders definitive conclusions challenging.

An evaluation of i) notable tumor volume reductions and ii) longest DOS, revealed that subjects whose tumors harbored FA/BRCA network mutations displayed the most favorable outcomes (DCR=71%; DOS=3.8 cycles). Genes in this network encode factors that directly or indirectly manage replication fork metabolism in response to RS. Importantly, these sensitivity trends were observed across multiple indications, suggesting a potential histology-independent sensitization.

The same underlying tumor genetics associated with enhanced sensitivity in this study were also observed in subjects treated in a clinical study of SRA737+low dose gemcitabine (LDG) (NCT02797977).

Within the FA/BRCA pathway, several notable tumor decreases occurred in subjects harboring two genetic alterations, frequently including a secondary mutation in a DDR checkpoint kinase gene (ATR, PRKDC). This phenomenon, which was also observed in the SRA737+LDG clinical study, suggests that overlapping, compound mutations may lead to elevated intrinsic RS and genomic instability, and/or be a consequence thereof.

The identification of both positive- and negative-selection genetic markers as determined in this clinical study may provide a focused ctDNA enrichment strategy for deployment in future clinical studies, potentially expediting prospective enrollment.

Overall, these data provide promising evidence of SRA737 anti-tumor activity and identify several gene networks associated with enhanced SRA737 sensitivity.

These findings suggest that additional RS, such as via extrinsic sources, may be desirable to generate durable objective responses with a highly selective Chk1i, as evidenced by anti-tumor activity demonstrated in the SRA737-02 clinical study, where potentiating LDG was combined with SRA737.

### Example 7: Phase 1/2 Clinical Study to Confirm Efficacy of SRA737 Combination Therapy in Select Tumors with Genetic Alterations that Confer Chk1 Sensitivity

An in-human clinical trial is conducted to confirm efficacy of SRA737 combination therapy methods of treatment and patient selection strategies disclosed herein for prospectively-selected genetically-defined subjects with tumor types known to have a high prevalence of genomic alterations expected to sensitize the tumor to Chk1 inhibition (“Cohort Expansion Phase”). In the Cohort Expansion Phase, approximately 20 prospectively-selected genetically-defined subjects are enrolled in each of 4 indication-specific cohorts: high-grade serous ovarian cancer (HGSOC), small cell lung cancer (SCLC), soft tissue sarcoma (STS), and cervical/anogenital cancer. Based on the PK data that established dosing resulting in an efficacious concentration of SRA737 (see Example 5), a starting dose level of 500 mg SRA737 and 100 mg/m² gemcitabine was used. The dosing regimen in some cases changes during the course of the trial. SRA737 capsules are taken on an empty stomach (subjects fast for at least 2 hours pre- and 1 hour post-administration), unless otherwise instructed.

Subjects have:
a. Histologically or cytologically proven advanced malignancy of the following types, for which no other conventional therapy is considered appropriate:
   i. High-grade serous ovarian cancer (HGSOC)
      1. Histologically confirmed high-grade serous ovarian, fallopian tube, or primary peritoneal cancer
      2. Platinum-resistant or refractory disease, or if the subject is intolerant to platinum therapy
   ii. Small cell lung cancer
      1. In general, have received at least 1 but no more than 3 prior regimens for advanced disease, unless otherwise approved by sponsor
   iii. Soft tissue sarcoma
      1. Including undifferentiated pleiomorphic sarcoma/malignant fibrous histiocytoma (MFH) (including high-grade spindle cell sarcoma/pleomorphic liposarcomas), leiomyosarcoma, and dedifferentiated liposarcomas. Other types of STS in some cases are eligible with sponsor's approval
      2. In general, have received at least 1 but no more than 3 prior regimens for advanced disease, unless otherwise approved by sponsor
   iv. Cervical/anogenital cancer
      1. Including all cervical carcinoma and advanced/metastatic squamous cell carcinoma of the anus, penis, vagina, and vulva
      2. In general, have received at least 1 but no more than 3 prior regimens for advanced disease, unless otherwise approved by sponsor
   v. Urothelial Carcinoma
      1. Histologically confirmed locally advanced and unresectable or metastatic urothelial carcinoma of the bladder, upper urinary tract or urethra
      2. In general, have received at least 1 but no more than 3 prior regimens for advanced disease.
b. Measurable disease per RECIST v1.1 (see below)
c. Subjects have predicted sensitivity to Chk1 inhibition based on factors including: genetic profiling of tumor tissue or ctDNA, HPV status, and germline BRCA1 and BRCA2 gene status. All subjects have genetic profiling from tumor tissue or ctDNA; profiling is performed prospectively if required to evaluate Chk1 sensitivity or otherwise performed retrospectively
   i. For subjects with HGSOC, documented somatic or germline BRCA1 and BRCA2 wild-type status confer eligibility without requirement for prospective genetic profiling. If documented BRCA status is not available, genetic profiling in some cases are performed prospectively to determine eligibility.
   ii. Subjects with SCLC are eligible without requirement for prospective genetic profiling on the basis of very high prevalence of cancer related alterations in the tumor suppressor genes (eg, TP53 and RB1) in this population.
   iii. For subjects with STS, and any others for whom genetic profiling is performed prospectively, eligibility is determined by the sponsor's review of genetic abnormalities detected in genes in the following categories:
      Key tumor suppressor genes regulating G1 cell cycle progression/arrest such as RB1, TP53, etc. For relevant cancers, positive human papilloma virus (HPV) status is also considered for eligibility.
         a. The DNA damage response pathway including ATM, CDK12, BRCA1, BRCA2, mismatch repair genetic alterations and/or high microsatellite instability.
         b. Genetic indicators of replicative stress such as gain of function/amplification of Chk1 or ATR or other related gene.
         c. Oncogenic drivers such as MYC, CCNE1, etc.
   iv. For subjects with anogenital cancer, known HPV positive status in some cases confers eligibility without requirement for prospective genetic profiling. If HPV status is not known or not positive, genetic profiling (or HPV testing where appropriate) in some cases is performed prospectively to determine eligibility. Subjects with cervical cancer or squamous cell carcinoma of the anus are eligible without requirement for prospective genetic profiling based on the very high prevalence of HPV positivity in these populations.

Alternatively, subjects have one of the histologically or cytologically proven advanced malignancies described above and tumor tissue or ctDNA evidence that their tumor harbors one or more mutations that are expected to confer sensitivity to Chk1 inhibition. Eligibility will be determined by the sponsor's review of genetic abnormalities detected in genes in the following categories:
a. Key tumor suppressor genes regulating G1 cell cycle progression/arrest such as RB1, TP53, etc. For relevant cancers, positive human papilloma virus (HPV) status is also considered for eligibility.
b. The DNA damage response pathway including ATM, CDK12, BRCA1, BRCA2, mismatch repair genetic alterations and/or high microsatellite instability.
c. Genetic indicators of replicative stress such as gain of function/amplification of Chk1 or ATR or other related gene.
d. Oncogenic drivers such as MYC, KRAS, etc.

Subjects are excluded based on the following criteria:
a. Received the following prior or current anticancer therapy in the timeframes noted prior to receiving SRA737 and have recovered from toxicity:
   i. Radiotherapy, chemotherapy, PARP inhibitors, other targeted therapies, or other IMPs within 2 weeks
   ii. Nitrosoureas or Mitomycin C within 6 weeks
   iii. Any prior treatment with a Chk1 inhibitor at any point or prior treatment with an ATR inhibitor within 6 months
b. No more than 3 previous treatment regimens for advanced disease (not applicable to HGSOC expansion cohort)
c. Other malignancy within the past 2 years, except for adequately treated tumors
d. If, in the opinion of the Investigator, the subject is highly likely to experience clinically significant myelosuppression
e. Ongoing toxic manifestations of previous treatments greater than NCI-CTCAE Grade 1
f. History of allergy to gemcitabine
g. New or progressing brain metastases. Subjects with brain metastases that have been asymptomatic and radiologically stable over an 8-week period and have not been treated with steroids during that time in some cases are included with approval from the sponsor.
h. High medical risk because of nonmalignant systemic disease
i. Serologically positive for hepatitis B, hepatitis C or HIV
j. Serious cardiac condition, left ventricular ejection fraction <45% at baseline, history of cardiac ischemia within the past 6 months, or prior history of cardiac arrhythmia requiring treatment, unless approved by the sponsor.
k. Prior bone marrow transplant or extensive radiotherapy to greater than 25% of bone marrow within the previous 8 weeks
l. Peanut allergy
m. QTcF>450 msec in adult makes and >470 msec in adult females
n. Impairment of gastrointestinal (GI) function or GI disease that in some cases significantly alter the absorption of SRA737
o. Inability to swallow capsules without chewing or crushing
p. Is a participant or plans to participate in another interventional clinical trial
q. Any other condition which in the Investigator's opinion would not make the subject a good candidate

All enrolled subjects who have measurable disease, receive at least 75% (Stage 1) or 83% (Stage 2) of SRA737 in 1 cycle (or the equivalent if the sponsor elects to evaluate an alternative dosing schedule), and have a baseline assessment of disease plus at least 1 postbaseline disease assessment are evaluated for response. All subjects who enroll into the Cohort Expansion Phase are evaluated for response if they have measurable disease, receive at least 1 cycle of study medication as defined above, have a baseline assessment of disease plus at least 1 postbaseline disease assessment and are confirmed as having met the genetic selection requirements.

In addition, subjects who have measurable disease and received at least 83% of SRA737 (if the sponsor elects to evaluate an alternative dosing schedule) in 1 cycle but developed PD, intolerable toxicity, or death prior to the postbaseline assessment are also evaluable and are classified as non-responders.

The analysis of all efficacy endpoints is based on the Response Evaluable Population and will be evaluated using RECIST v1.1 criteria, as described below.

Other endpoints include: Duration of response (DOR), Disease control rate (DCR), Time to response (TTR), PFS, Time to Progression (TTP), OS. Other exploratory objectives are described in Table 11.

TABLE 11
EXPLORATORY OBJECTIVES AND ENDP.INTS

| Exploratory Objectives | Endpoints |
|---|---|
| To assess the relationship between response and | Objective response rate as measured by RECIST |
| the presence of selected genetic alternations | v1.1 and gene alterations in tumor tissue or ctDNA |
| detected in tumor tissue or circulating tumor | at baseline as measured by next generation |
| deoxyribonucleic acid (ctDNA). | sequencing (NGS). |
| To explore possible clinical predictors of | Characteristics such as performance status, prior |
| outcomes, | therapy, indication and other known or potential |
| | prognostic or predictive factors. |
| To investigate the PDn of SRA737 in combination | Proof of target engagement and changes in |
| with gemcitabine in tumor tissue. | mechanism of action biomarkers between baseline |
| | and on treatment with SRA737, including, but not |
| | limited to: pSer296 Chk1, pS317 Chk1, and total |
| | Chk1. |
| To investigate the PDn of SRA737 in combination | Proof of target engagement and changes in |
| with gemcitabine in surrogate tissues such as blood | mechanism of action biomarkers between baseline |
| or peripheral blood mononuclear cell (PBMCs). | and on treatment with SRA737, including but not |
| | limited to: Comet assay, pS296 Chkl, pS317 |
| | Chk1, pS345 Chk1, total Chk1, gammaH2AX and |
| | RAD51. |

Additional trials are conducted with SRA737 in combination with other therapies, including administering a chemotherapeutic agent, administering an antibody or antibody fragment, administering a radiation treatment, administering an external inducer of replication stress, or administering a combination thereof. Other trials are conducted with SRA737 in combination with other therapies, including administering olaparib, niraparib, rucaparib, talazoparib, cisplatin, a ribonucleotide reductase inhibitor, etoposide, SN-38/CPT-11, mitomycin C, or combinations thereof.

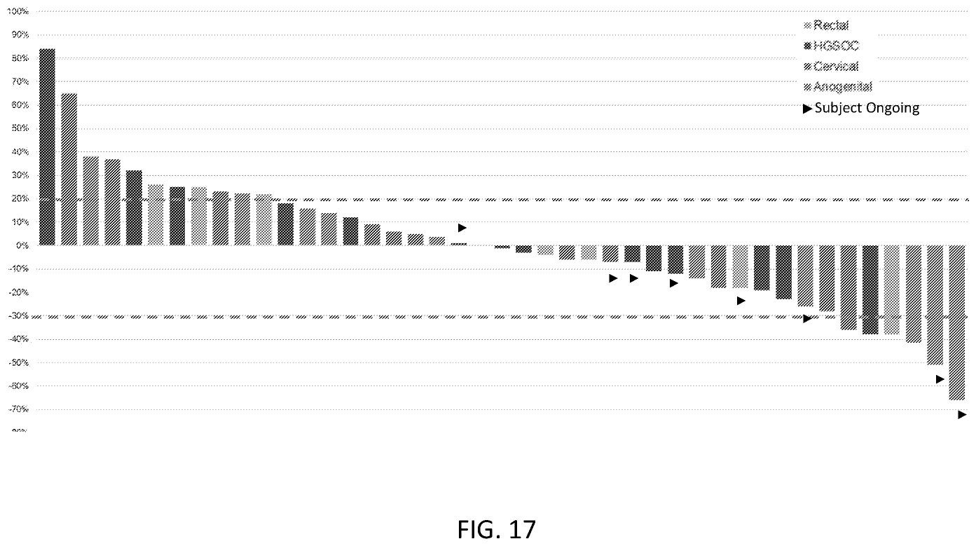

The results of this study confirm the efficacy of SRA737 combination therapy for the treatment of tumors with known genetic alterations expected to confer sensitivity to Chk1 inhibition (FIG. 17). Proof-of-concept clinical activity has been seen in tumor types such as anogenital (e.g., anal), cervical and rectal. The combination of LDG and SRA737 has been well tolerated. This first-in-human clinical study provides proof-of-concept that sub-therapeutic LDG effectively potentiates SRA737 anti-tumor activity.

### RECIST Criteria

Assessment of disease response in this study are performed according to the revised RECIST criteria v1.1. RECIST criteria are described in greater detail in Eisenhauer, et al. (New response evaluation criteria in solid tumours: Revised RECIST guideline (version 1.1). Eur J Cancer [Internet]. 2009), herein incorporated by reference for all it teaches.

At baseline, tumor lesions/lymph nodes are generally categorized measurable or non-measurable as follows:

Measurable

Tumor lesions: are generally accurately measured in at least one dimension (longest diameter in the plane of measurement is to be recorded) with a minimum size of:
10 mm by CT scan (CT scan slice thickness no greater than 5 mm; see Appendix II in Eisenhauer, et al., [Eisenhauer, 2009] on imaging guidance)
10 mm caliper measurement by clinical exam (lesions which cannot be accurately measured with calipers should be recorded as non-measurable)
20 mm by chest X-ray

Malignant lymph nodes: To be considered pathologically enlarged and measurable, a lymph node is generally 15 mm in the short axis when assessed by CT scan (CT scan slice thickness recommended to be no greater than 5 mm). At baseline and in follow-up, only the short axis is generally measured and followed.

Non-Measurable

All other lesions, including small lesions (longest diameter <10 mm or pathological lymph nodes with ≥10 to <15 mm short axis) as well as truly non-measurable lesions. Lesions considered truly non-measurable generally include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Bone lesions, cystic lesions, and lesions previously treated with local therapy require particular comment:

Bone Lesions:
Bone scan, PET scan or plain films are generally not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques such as CT or MRI are generally considered as measurable lesions if the soft tissue component meets the definition of measurability described above.
Blastic bone lesions are generally non-measurable.

Cystic Lesions:
Lesions that meet the criteria for radiographically defined simple cysts are generally not considered as malignant lesions (neither measurable nor non-measurable) since they are, by definition, simple cysts.
‘Cystic lesions’ thought to represent cystic metastases are generally considered as measurable lesions, if they meet the definition of measurability described above. However, if non-cystic lesions are present in the same subject, these are preferred for selection as target lesions.

Lesions with Prior Local Treatment:
Tumor lesions situated in a previously irradiated area, or in an area subjected to other loco-regional therapy, are usually not considered measurable unless there is demonstrated progression in the lesion. Study protocols generally detail the conditions under which such lesions are generally considered measurable.

Method of Assessment

All measurements are generally recorded in metric notation, using calipers if clinically assessed. All baseline evaluations are generally performed as close as possible to the treatment start and never more than 4 weeks before the beginning of the treatment.

The same method of assessment and the same technique are generally used to characterize each identified and reported lesion at baseline and during follow-up. Imaging based evaluation are generally always done rather than clinical examination unless the lesion(s) being followed cannot be imaged but are assessable by clinical exam.

Clinical lesions are generally considered measurable when they are superficial and ≥10 mm diameter as assessed using calipers (e.g. skin nodules). For the case of skin lesions, documentation by color photography including a ruler to estimate the size of the lesion is suggested. As noted above, when lesions can be evaluated by both clinical exam and imaging, imaging evaluation is generally undertaken since it is more objective and in some cases is also reviewed at the end of the study.

Chest CT is generally preferred over chest X-ray, particularly when progression is an important endpoint, since CT is more sensitive than X-ray, particularly in identifying new lesions. However, in some cases, lesions on chest X-ray are considered measurable if they are clearly defined and surrounded by aerated lung

CT is generally the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion is twice the slice thickness. MRI is also acceptable in certain situations (e.g. for body scans). More details concerning the use of both CT and MRI for assessment of objective tumor response evaluation are provided in the publication from Eisenhauer et al.

Ultrasound is generally not useful in assessment of lesion size and is generally not used as a method of measurement. Ultrasound examinations, in general, cannot be reproduced in their entirety for independent review at a later date and, because they are operator dependent, it generally cannot be guaranteed that the same technique and measurements will be taken from one assessment to the next (described in greater detail in Eisenhauer, et al. (2009). If new lesions are identified by ultrasound in the course of the study, confirmation by CT or MRI is generally advised. If there is concern about radiation exposure at CT, MRI in some cases is used instead of CT in selected instances.

The utilization of endoscopy and laparoscopy techniques for objective tumor evaluation is generally not advised. However, they are, in general, useful to confirm complete pathological response when biopsies are obtained or to determine relapse in trials where recurrence following complete response or surgical resection is an endpoint.

Tumor markers alone are generally not used to assess objective tumor response. If markers are initially above the upper normal limit, however, they are generally normalized for a subject to be considered in complete response.

Cytology and histology are generally used to differentiate between PR and CR in rare cases if required by protocol (for example, residual lesions in tumor types such as germ cell tumors, where known residual benign tumors can remain). When effusions are known to be a potential adverse effect of treatment (e.g. with certain taxane compounds or angiogenesis inhibitors), the cytological confirmation of the neoplastic origin of any effusion that appears or worsens during treatment are generally considered if the measurable tumor has met criteria for response or stable disease in order to differentiate between response (or stable disease) and progressive disease.

Tumor Response Evaluation

To assess objective response or future progression, the overall tumor burden at baseline is generally estimated and used as a comparator for subsequent measurements. Measurable disease is generally defined by the presence of at least one measurable lesion.

When more than one measurable lesion is present at baseline all lesions up to a maximum of five lesions total (and a maximum of two lesions per organ) representative of all involved organs are generally identified as target lesions and are recorded and measured at baseline (this means in instances where subjects have only one or two organ sites involved a maximum of two and four lesions respectively are recorded). Target lesions are generally selected on the basis of their size (lesions with the longest diameter) and are generally representative of all involved organs, but in addition are generally those that lend themselves to reproducible repeated measurements. In some cases, the largest lesion does not lend itself to reproducible measurement in which circumstance the next largest lesion which can be measured reproducibly is generally selected, as exemplified in FIG. 3 Eisenhauer, et al. (2009).

Lymph nodes merit special mention since they are normal anatomical structures which in some cases are visible by imaging even if not involved by tumor. Pathological nodes which are defined as measurable and in some cases are identified as target lesions, in general, meets the criterion of a short axis of ≥15 mm by CT scan. Only the short axis of these nodes generally contributes to the baseline sum. The short axis of the node is generally the diameter normally used by radiologists to judge if a node is involved by solid tumor. Nodal size is normally reported as two dimensions in the plane in which the image is obtained (for CT scan this is almost always the axial plane; for MRI the plane of acquisition in some cases are axial, sagital or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis ≥10 mm but <15 mm) are generally considered non-target lesions. Nodes that have a short axis <10 mm are generally considered non-pathological and are generally not recorded or followed.

A sum of the diameters (longest for non-nodal lesions, short axis for nodal lesions) for all target lesions is generally calculated and reported as the baseline sum diameters. If lymph nodes are to be included in the sum, then as noted above, only the short axis is added into the sum. The baseline sum diameters are generally used as reference to further characterize any objective tumor regression in the measurable dimension of the disease.

All other lesions (or sites of disease) including pathological lymph nodes are generally identified as non-target lesions and are generally recorded at baseline. Measurements are generally not required and these lesions are generally followed as ‘present’, ‘absent’, or in rare cases ‘unequivocal progression’ (more details to follow). In addition, it is possible to record multiple non-target lesions involving the same organ as a single item on the case record form (e.g. ‘multiple enlarged pelvic lymph nodes’ or ‘multiple liver metastases’).

Response Criteria

Complete Response (CR): Disappearance of all target lesions. Any pathological lymph nodes (whether target or non-target) are reduced in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum (this includes the baseline sum if that is the smallest on study). In addition to the relative increase of 20%, the sum generally demonstrates an absolute increase of at least 5 mm. (Note: the appearance of one or more new lesions is generally also considered progression).

Stable Disease (SD): Neither sufficient shrinkage to qualify for PR nor sufficient increase to qualify for PD, taking as reference the smallest sum diameters.

Lymph nodes identified as target lesions generally record the actual short axis measurement (measured in the same anatomical plane as the baseline examination), generally even if the nodes regress to below 10 mm. This means that when lymph nodes are included as target lesions, the ‘sum’ of lesions in some cases are not be zero even if complete response criteria are met, since a normal lymph node is generally defined as having a short axis of <10 mm. Case report forms or other data collection methods in some cases are therefore designed to have target nodal lesions recorded in a separate section where, in order to qualify for CR, each node generally achieves a short axis <10 mm. For PR, SD and PD, the actual short axis measurement of the nodes is preferably included in the sum of target lesions.

While on study, all lesions (nodal and non-nodal) recorded at baseline generally record their actual measurements at each subsequent evaluation, even when very small (e.g. 2 mm). However, sometimes lesions or lymph nodes which are recorded as target lesions at baseline become so faint on CT scan that the radiologist in some cases does not feel comfortable assigning an exact measure and in some cases report them as being ‘too small to measure’. When this occurs it is, in general, important that a value is recorded on the case report form. If it is the opinion of the radiologist that the lesion has likely disappeared, the measurement is generally recorded as 0 mm. If the lesion is believed to be present and is faintly seen but too small to measure, a default value of 5 mm is generally assigned. (Note: It is, in general, less likely that this rule is used for lymph nodes since they usually have a definable size when normal and are frequently surrounded by fat such as in the retroperitoneum; however, if a lymph node is believed to be present and is faintly seen but too small to measure, a default value of 5 mm is generally assigned in this circumstance as well). This default value is derived from the 5 mm CT slice thickness (but generally is not changed with varying CT slice thickness). The measurement of these lesions is potentially non-reproducible, therefore providing this default value generally prevents false responses or progressions based upon measurement error. To reiterate, however, if the radiologist is able to provide an actual measure, that is generally recorded, even if it is below 5 mm.

When non-nodal lesions ‘fragment’, the longest diameters of the fragmented portions are generally added together to calculate the target lesion sum. Similarly, as lesions coalesce, a plane between them are generally maintained that would aid in obtaining maximal diameter measurements of each individual lesion. If the lesions have truly coalesced such that they are no longer separable, the vector of the longest diameter in this instance generally is the maximal longest diameter for the ‘coalesced lesion’.

While some non-target lesions in some cases are actually measurable, they generally are not measured and instead are generally assessed only qualitatively at the time points specified in the protocol.

Complete Response (CR): Disappearance of all non-target lesions and normalization of tumor marker level. All lymph nodes are non-pathological in size (<10 mm short axis).

Non-CR/Non-PD: Persistence of one or more non-target lesion(s) and/or maintenance of tumor marker level above the normal limits.

Progressive Disease (PD): Unequivocal progression (see comments below) of existing non-target lesions. (Note: the appearance of one or more new lesions is also considered progression).

When the subject also has measurable disease, to achieve ‘unequivocal progression’ on the basis of the non-target disease, there generally is an overall level of substantial worsening in non-target disease such that, even in presence of SD or PR in target disease, the overall tumor burden has increased sufficiently to merit discontinuation of therapy. A modest ‘increase’ in the size of one or more non-target lesions is usually not sufficient to quality for unequivocal progression status. The designation of overall progression solely on the basis of change in non-target disease in the face of SD or PR of target disease is generally therefore extremely rare.

A subject having only non-measurable disease arises in some Phase III trials when it is not a criterion of study entry to have measurable disease. The same general concepts apply here as noted above, however, in this instance there is no measurable disease assessment to factor into the interpretation of an increase in non-measurable disease burden. Because worsening in non-target disease is generally not easily quantified (by definition: if all lesions are truly non-measurable) a useful test that can generally be applied when assessing subjects for unequivocal progression is to consider if the increase in overall disease burden based on the change in non-measurable disease is comparable in magnitude to the increase that would be required to declare PD for measurable disease: i.e. an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or in some cases are described in protocols as ‘sufficient to require a change in therapy’. If ‘unequivocal progression’ is seen, the subject is generally considered to have had overall PD at that point. While it would be ideal to have objective criteria to apply to non-measurable disease, the very nature of that disease makes it generally very difficult to do so; therefore the increase generally is substantial.

The appearance of new malignant lesions generally denotes disease progression; therefore, some comments on detection of new lesions are generally important. There are generally no specific criteria for the identification of new radiographic lesions; however, the finding of a new lesion generally is unequivocal: i.e., generally not attributable to differences in scanning technique, change in imaging modality or findings thought to represent something other than tumor (for example, some ‘new’ bone lesions in some cases are simply healing or flare of pre-existing lesions). This is particularly important when the subject's baseline lesions show partial or complete response. For example, necrosis of a liver lesion is frequently reported on a CT scan report as a ‘new’ cystic lesion, which it generally is not.

A lesion identified on a follow-up study in an anatomical location that was not scanned at baseline is generally considered a new lesion and generally indicates disease progression. An example of this is the subject who has visceral disease at baseline and while on study has a CT or MRI brain ordered which reveals metastases. The subject's brain metastases are generally considered to be evidence of PD even if he/she did not have brain imaging at baseline.

If a new lesion is equivocal, for example because of its small size, continued therapy and follow-up evaluation generally clarifies if it represents truly new disease. If repeat scans confirm there is definitely a new lesion, then progression is generally declared using the date of the initial scan.

While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging are generally identified according to the following algorithm:

a. Negative FDG-PET at baseline, with a positive* FDG-PET at follow-up is generally a sign of PD based on a new lesion. (* A ‘positive’ FDG-PET scan lesion generally means one which is FDG avid with an uptake greater than twice that of the surrounding tissue on the attenuation corrected image.)

b. No FDG-PET at baseline and a positive FDG-PET at follow-up:
If the positive FDG-PET at follow-up corresponds to a new site of disease confirmed by CT, this is generally PD.
If the positive FDG-PET at follow-up is not confirmed as a new site of disease on CT, additional follow-up CT scans are generally performed to determine if there is truly progression occurring at that site (if so, the date of PD will be the date of the initial abnormal FDG-PET scan). A ‘positive’ FDG-PET scan lesion generally means one which is FDG avid with an uptake greater than twice that of the surrounding tissue on the attenuation corrected image.
If the positive FDG-PET at follow-up corresponds to a pre-existing site of disease on CT that is not progressing on the basis of the anatomic images, this is generally not PD.

Evaluation of Best Overall Response

The best overall response is generally the best response recorded from the start of the study treatment until the end of treatment. Should a response not be documented until after the end of therapy in this trial, post-treatment assessments generally are considered in the determination of best overall response as long as no alternative anti-cancer therapy has been given. The subject's best overall response assignment generally depends on the findings of both target and non-target disease and generally also takes into consideration the appearance of new lesions.

It is generally assumed that at each protocol-specified time point, a response assessment occurs. Table 12 provides a summary of the overall response status calculation at each time point for subjects who have measurable disease at baseline.

When subjects have non-measurable (therefore non-target) disease only, Table 13 is generally used.

When no imaging/measurement is done at all at a particular time point, the subject is generally not evaluable (NE) at that time point. If only a subset of lesion measurements are made at an assessment, usually the case is generally also considered NE at that time point, unless a convincing argument is made that the contribution of the individual missing lesion(s) does not change the assigned time point response. This would be most likely to happen in the case of PD. For example, if a subject had a baseline sum of 50 mm with three measured lesions and at follow-up only two lesions were assessed, but those gave a sum of 80 mm, the subject has generally achieved PD status, regardless of the contribution of the missing lesion.

The best overall response is generally determined once all the data for the subject is known.

Best response determination in trials where confirmation of complete or partial response is generally not required: Best response in these trials is generally defined as the best response across all time points (for example, a subject who has SD at first assessment, PR at second assessment, and PD on last assessment has a best overall response of PR). When SD is believed to be best response, it, in general, also meets the protocol specified minimum time from baseline. If the minimum time is not met when SD is otherwise the best time point response, the subject's best response generally depends on the subsequent assessments. For example, a subject who has SD at first assessment, PD at second and does not meet minimum duration for SD, will have a best response of PD. The same subject lost to follow-up after the first SD assessment is generally considered inevaluable.

When nodal disease is included in the sum of target lesions and the nodes decrease to ‘normal’ size (<10 mm), in some cases they still have a measurement reported on scans. This measurement is generally recorded even though the nodes are normal in order not to overstate progression should it be based on increase in size of the nodes. As noted earlier, this means that subjects with CR in some cases do not have a total sum of ‘zero’ on the case report form (CRF).

Subjects with a global deterioration of health status requiring discontinuation of treatment without objective evidence of disease progression at that time generally are reported as ‘symptomatic deterioration’. Every effort is generally made to document objective progression even after discontinuation of treatment. Symptomatic deterioration is generally not a descriptor of an objective response: it is a reason for stopping study therapy. The objective response status of such subjects is generally determined by evaluation of target and non-target disease as shown in Tables 12 and 13.

Conditions that define ‘EP, early death and inevaluability’ are study specific and are generally clearly described in each protocol (depending on treatment duration, treatment periodicity).

In some circumstances it is difficult to distinguish residual disease from normal tissue. When the evaluation of complete response depends upon this determination, it is generally recommended that the residual lesion be investigated (fine needle aspirate/biopsy) before assigning a status of complete response. In some cases, FDG-PET is used to upgrade a response to a CR in a manner similar to a biopsy in cases where a residual radiographic abnormality is thought to represent fibrosis or scarring.

For equivocal findings of progression (e.g. very small and uncertain new lesions; cystic changes or necrosis in existing lesions), treatment in some cases continues until the next scheduled assessment. If at the next scheduled assessment, progression is confirmed, the date of progression generally is the earlier date when progression was suspected.

TABLE 12
Time point response: subjects with target (+/− non-target) disease

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or not | No | PR |
| | all evaluated | | |
| SD | Non-PD or not | No | SD |
| | all evaluated | | |
| Not all evaluated | Non-PD | No | NE |
| PD | Any | Yes or No | PD |
| Any | PD | Yes or No | PD |
| Any | Any | Yes | PD |

CR = complete response, PR = partial response, SD = stable disease PD = progressive disease, and NE = not evaluable.

TABLE 13
Time point response: subjects with non-target disease only

| Non-target lesions | New lesions | Overall response |
|---|---|---|
| CR | No | CR |
| Non-CR/non-PD | No | Non-CR/non-PD(a) NE |
| Not all evaluated | No | PD |
| Unequivocal PD | Yes or No | PD |
| Any | Yes | PD |

CR = complete response, PD = progressive disease and NE = not evaluable.
(a) ‘Non-CR/non-PD’ is preferred over ‘stable disease’ for non-target disease since SD is increasingly used as endpoint for assessment of efficacy in some trials so to assign this category when no lesions can be measured is not advised.

Duration of Response

The duration of overall response is generally measured from the time measurement criteria are first met for CR/PR (whichever is first recorded) until the first date that recurrent or progressive disease is recorded on study).

The duration of overall complete response is generally measured from the time measurement criteria are first met for CR until the first date that recurrent disease is objectively documented.

Stable disease is generally measured from the start of the treatment (in randomized trials, from date of randomization) until the criteria for progression are met, taking as reference the smallest sum on study (if the baseline sum is the smallest, this is the reference for calculation of PD).

### Results

This signal-seeking Phase 1/2 study (NCT02797977) was designed to investigate the safety and tolerability of SRA737 in combination with sub-therapeutic (low dose) gemcitabine (LDG), as well as to evaluate preliminary anti-tumor activity in tumors with genetic alterations that can confer increased intrinsic RS and Chk1i sensitivity. Prospective genetic screening was performed to identify and select subjects harboring one or more of these genetic alterations. The study was designed as a broad survey to explore the association between various sources of intrinsic replication stress and SRA737+LDG anti-tumor activity in the expansion phase, in order to delineate potential genetic signatures and/or tumor indications that might warrant additional therapeutic investigation.

### Inducers of Replication Stress Potentiate Chk1i

Replication stress (RS) is manifested by the slowing and stalling of replication forks which results in fragile exposed single-stranded DNA that is prone to damage. Chk1 plays an essential role in the preservation of replication fork stability and the cellular response to RS in order to maintain genomic stability. RS-driver genes can be divided into several functional categories including G1/S tumor suppressors, oncogenes and DNA repair genes. A comprehensive candidate “RS-driver” gene list was developed based on a compilation of available preclinical and clinical data, delineated into these functional categorizations and used for prospective subject selection to enable direct clinical exploration of these potential Chk1i-sensitizing genetics.

To determine whether additional extrinsic RS might augment the intrinsic RS afforded by tumor genetics, we treated subjects with the potent RS-inducer LDG to further increase tumor reliance on Chk1, with the intention of potentiating the anti-tumor activity of SRA737.

### Methods

In the Dose Escalation phase, subjects with solid tumors in cohorts of 3 to 6 subjects received escalating doses of SRA737 in combination with varying sub-therapeutic doses of gemcitabine. SRA737 was administered for 2 days after LDG administration on days 1, 8 and 15 of a 28 day cycle. A lead-in dose for pharmacokinetic (PK) analysis was performed 4-7 days prior to Cycle 1 (C1).

Expansion cohorts were contemporaneously initiated when circulating plasma concentrations of SRA737 exceeded the minimum effective concentration of SRA737 modelled from murine efficacy studies.

Thereafter, experience gained in the ongoing Dose Escalation phase informed dosing in the expansion cohorts. The Cohort Expansion phase enrolled subjects with genetically defined tumors that harbored genomic alterations hypothesized to confer sensitivity to Chk1 inhibition, which were prospectively selected by next-generation sequencing (FoundationOne). Subjects with the following tumors were eligible for enrollment: i) soft tissue sarcoma, ii) high grade serous ovarian (HGSOC), iii) small cell lung, and iv) anogenital/cervical cancers. Subjects with anogenital or cervical cancer were eligible for enrollment without prospective genetic profiling based on the near ubiquitous prevalence of HPV-positivity in this population.

This signal seeking Phase1/2 study was generally conducted in specialty Phase 1 cancer units. Subjects ≥18 years of age with an ECOG performance status of 0-1; measurable disease (per RECIST v1.1) and with archival tumor tissue (or willingness to consent to a biopsy) were eligible to participate in the study. Expansion phase subjects variously received 1 to 3 prior regimens for advanced disease; for HGSOC, there was no limit on prior regimens.

Dose limiting toxicities (DLTs) were assessed following a PK lead-in dose, and throughout Cycle 1 of treatment, and were defined as adverse events (AEs) highly probably or probably related to study drug (SRA737 and gemcitabine): grade 4 neutropenia or thrombocytopenia (lasting >7 days): febrile neutropenia; thrombocytopenia (grade ≥3) with bleeding (grade ≥3); non-hematologic toxicity (grade ≥3) or inability to receive 5 of the 6 (83%) planned doses of SRA737 or all doses of gemcitabine in cycle 1 due to study drug (SRA737 and gemcitabine)-related toxicity.

Preliminary anti-tumor activity was assessed by target tumor response and overall response in accordance with RECIST v1.1.

Genomics: Although genomic information was required to enter the clinical study, several subjects enrolled with failed FMI reports (Foundation Medicine), thus leaving their genomics unknown. Several patient reports have yet to be received and thus are currently unknown. A majority of sequenced patients were sequenced using FMI assays only; however several patients were sequenced using FMI and the Guardant 360 panel (one patient was only sequenced by G360). For the most part, the assays were highly concordant, however the Guardant360 panel has fewer genes than FMI and thus some genes could be “missed”

HPV Status: When possible, HPV status was determined using standard IHC based methods. Foundation Medicine provides data on the HPV status of sequenced tumors. Only HPV strains 6, 11, 16 and 18 are sequenced; 16 and 18 are the most common strains in cervical (75%) and anal cancer (79%) but several other subtypes are implicated as high-risk/cancer related (HPV-33 is about 5% of anal cancers).

### Subject Characteristics and Dose Evaluation

A total of 55 subjects received SRA737 in 13 escalation cohorts at doses of 40 to 600 mg SRA737 variously combined with LDG doses of 50 to 300 mg/m2. No protocol-defined dose limiting toxicities (DLTs) were observed, but intolerability was notably evident at the highest doses tested. The pharmacokinetic profile of SRA737 revealed AUC0-24 and Cmax of 3550 ng-h/mL and 548 ng/mL at 150 mg SRA737. At this dose, the Cmin (52 ng/mL) exceeded that determined in preclinical models to be effective.

Enrollment into the expansion cohorts was initiated at 500 mg SRA737+100 mg/m2 LDG.

Based on overall tolerability, the recommended dose to be employed in the expansion cohorts was determined to be 500 mg SRA737+250 mg/m2 LDG (RP2D).

Of 335 subjects prospectively identified, 204 were screened for genetic alterations associated with Chk1 sensitivity. Of these subjects 176 (86%) met genetic eligibility criteria, and 86 were enrolled into the four expansion cohorts.

### Safety

Treatment-emergent adverse events (TEAEs) were reported in 137 (99%) subjects and 131 subjects (94%) experienced at least one study drug (SRA737 and/or gemcitabine)-related event. The majority of TEAEs were mild to moderate in severity (91% Grade 1/Grade 2). The most common TEAEs were nausea (60%), vomiting (50%), diarrhea (45%), fatigue (43%), anemia (33%) and pyrexia (31%). The most common ≥Grade 3 TEAEs were neutropenia (9%), anemia and ALT increased (6% each), AST increased (5%), thrombocytopenia (4%), hyponatremia and lymphopenia (3% each). The most common ≥Grade 3 study drug (SRA737 and/or gemcitabine)-related TEAEs were neutropenia (9%), ALT increased (5%), thrombocytopenia and AST increased (4%) and anemia (3%). Five Grade 5 TEAEs were reported up to 30-days post last treatment; none were considered related to SRA737. Median duration of exposure: 2 cycles (Range <1 to 13 cycles). There was no evidence of emergent or cumulative toxicity and/or declining tolerability with up to 13 cycles.

TABLE 14
Summary of Treatment-emergent adverse events (TEAEs).
TEAEs regardless of the investigator's assessment
of causality; data cutoff Mar. 23, 2019

| | Occurring in ≥20% | |
|---|---|---|
| | subjects | ≥ Grade 3 |
| TEAE | N = 139, n (%) | N = 139, n (%) |
| Subjects with any TEAE | 137 (98.6%) | 88 (63.4%) |
| Nausea | 83 (59.7%) | 1 (0.7%) |
| Vomiting | 70 (50.4%) | 3 (2.2%) |
| Diarrhea | 63 (45.3%) | 3 (2.2%) |
| Fatigue | 60 (43.2%) | 3 (2.2%) |
| anemia | 46 (33.1%) | 8 (5.8%) |
| pyrexia | 43 (30.9%) | 1 (0.7%) |
| neutropenia | 36 (25.9%) | 13 (9.4%) |
| decreased appetite | 33 (23.7%) | 0 |
| thrombocytopenia | 33 (23.7%) | 5 (3.6%) |
| ALT increased | 31 (22.3%) | 8 (5.8%) |
| AST increased | 28 (20.1%) | 7 (5.0%) |
| constipation | 28 (20.1%) | 2 (1.4%) |

### SRA737-02 Results

141 subjects received treatment with SRA737 (+LDG) across both escalation and expansion cohorts. The largest number of subjects (n=35) were enrolled in the anogenital/cervical cancer cohort with the next largest numbers in the HGSOC (n=28) and SCLC (n=23) cohorts.

The mean number of prior treatment regimens across all tumor types was 2.8 with the HGSOC cohort subjects having received a mean of 4.2 prior regimens consistent with a heavy pretreated cohort.

The median duration on treatment for all subjects was 2 cycles with a maximal treatment duration of 13 cycles; 22 subjects remained on study treatment at the time of the data cut off (3 May 2019)

41 subjects had a best response of Stable Disease (SD); durable SD lasting ≥4 months was recorded in 32 subjects and was observed in all expansion cohorts.

Of the subjects treated, 81/141 (57%) were considered evaluable for RECIST target tumor response and of these, 54 had an available genetic profile.

Partial responses (PR) were observed in 6 subjects (FIG. 17). These included 3 subjects with anogenital cancer and one subject each with rectal, cervical, and ovarian cancer. In general, tumor responses were first recorded at the end of Cycle 2 (first on-study scan).

The heavily pre-treated HGSOC cohort (˜4 prior lines) demonstrated directionally favorable disease control (DCR=67%); maximal tumor reduction noted was 38% (PR).

The ORR for subjects with squamous anogenital/cervical cancer was 22% (4/18).

Anogenital cancer was identified as the indication most sensitive to SRA737+LDG in this clinical study. (ORR=30%; DCR=60%)

The magnitude of target tumor decrease in anogenital tumors was notable; as of the data cutoff two subjects achieved ongoing decreases of −66% and −51% respectively, and a third subject achieved a decrease of −41%.

In addition, several subjects with anogenital cancer had noteworthy durations of response: 5/10 (50%) subjects remained on study treatment for ≥4 months with a maximal duration of ˜11 months. As of the data cutoff, study treatment was ongoing in 5 subjects (5/10; 50%).

In keeping with the signal seeking objective of this study, tumor responses were further examined with respect to the genetic profiles determined for the tumor types enrolled and treated.

RS-driver genes encompassing functional categories (G1/S, Oncogenes, DNA repair genes) were surveyed across multiple indications to identify gene networks and/or individual genes that enriched for sensitivity to treatment with SRA737+LDG

The number of subjects with tumor alterations in the selected gene networks varied based on i) the occurrence of particular genetic alterations within the indications explored and ii) enrollment metrics

Mutations in the RAS gene network were associated with relatively poor response.

In contrast, alterations in the PI3K gene network (PIK3CA, AKT or PTEN mutations) resulted in a robust 75% DCR and PRs in two subjects.

Similarly, genetic alterations in multiple components of the FA/BRCA gene network resulted in an 81% DCR and a 25% response rate (RR).

Alterations in the CCNE network were associated with favorable DCR (67%) and a tumor response was observed in a subject with HGSOC, although defined CCNE network observations were based on limited data (n=6)

Several of the robust responses observed in this study were associated with genomic alterations in the FA/BRCA network, frequently involving a secondary alteration in one of two DDR checkpoint kinase genes (ATR, PRKDC).

The findings suggest that multiple replication fork-associated mutations may exacerbate intrinsic RS and genomic instability, and/or be a consequence thereof. Consistent with this hypothesis, subjects who had available genetics that achieved PR were generally determined to have alterations in multiple gene networks.

Notably, elevated tumor mutational burden (TMB) was associated with certain tumor responses, particularly in subjects with anogenital and rectal cancer. Specifically, 3 of 4 subjects with anogenital cancer presenting with elevated TMB had robust responses, encompassing some of the most profound tumor decreases observed in the SRA737-02 study.

### Summary

In this first-in-human trial of SRA737+LDG, the RP2D (recommended phase II dose) was determined to be 500 mg SRA737 plus 250 mg/m² gemcitabine. Consistent with the RS-inducing properties of LDG, this combination utilized a gemcitabine dose substantially below (10-25%) standard of care dose levels, e.g., a sub-therapeutic dose of gemcitabine. The combination of SRA737+LDG was safe and well tolerated.

In aggregate, the safety and efficacy data determined in this study support that SRA737+LDG is readily conducive to development as a standalone therapy and appears potentially combinable with other therapeutics.

This signal-seeking study surveyed broadly across tumor indications and tumor RS-driver genetics to identify potential SRA737-sensitive settings in the context of the potentiating effect of the extrinsic RS-inducer, LDG. Preliminary evidence suggests several intrinsic sources of RS combined with LDG significantly enhances Chk1 activity. For example, mutations in the PI3K gene network correlated with both tumor responses and robust DCR (75%).

FA BRCA network mutations were associated with the most favorable outcomes in this study (ORR=25%; DCR=81%). The FA/BRCA gene network encodes a series of Fanconi Anemia and other proteins involved directly or indirectly in replication fork metabolism and management of RS.

The sensitivity of SRA737+LDG associated with mutations in the PI3K and FA/BRCA gene networks observed in this study were consistent with similar findings from the SRA737 monotherapy clinical study (NCT02797964), reinforcing that these alterations act as Chk1i-sensitizing genetic contexts. Moreover, these network alterations occurred across several tumor indications, suggesting potential histology-independent sensitization.

Notably, subjects whose tumors harbored multiple gene network alterations tended to have more favorable tumor reductions and longer DOS (duration on study). This phenomenon, which was also observed in the SRA737 monotherapy clinical study, suggests that overlapping, compound mutations may lead to elevated intrinsic RS and genomic instability, and/or be a consequence thereof.

A preliminary correlation of noteworthy tumor responses with elevated tumor mutational burden (TMB) was also observed, particularly in the anogenital cohort (3 of 4 patients achieved notable tumor decreases). Elevated TMB (e.g., intermediate TMB levels or higher) is consistent with increased genomic instability and represents a possible enrichment strategy.

Overall, these data provide clear evidence of SRA737+LDG anti-tumor activity. Multiple partial responses were observed, generally first recorded at the first on-study scan (end of cycle 2).

Efficacy was determined across several tumor indications, with the strongest efficacy signal observed in the squamous anogenital/cervical cohort (ORR=22%; DCR=50%).

Specifically, striking and unambiguous anti-tumor activity was observed in subjects with advanced anogenital cancer (ORR=30%; DCR=60%), encompassing noteworthy tumor decreases (e.g. ˜66% tumor decrease; resolution of pleural effusion) and promising durations of treatment (e.g. ˜11 months).

Second line metastatic anogenital cancer represents a significant unmet medical need, with no approved therapies and a significantly abrogated life expectancy. These promising data indicate that SRA737+LDG could be an efficacious treatment option for these patients.

### Example 10: Assessment of Genetic Abnormalities and Tumor Mutational Burden

Genetic abnormalities and TMB were assessed in tumor samples from individual patients using the FoundationOne CDx™ assay.

FoundationOne CDx™ (F1CDx) is a next generation sequencing based in vitro diagnostic device for detection of substitutions, insertion and deletion alterations (indels), and copy number alterations (CNAs) in up to 324 genes or more and select gene rearrangements, as well as genomic signatures including microsatellite instability (MSI) and tumor mutational burden (TMB) using DNA isolated from formalin-fixed paraffin embedded (FFPE) tumor tissue specimens. The test can be used as a companion diagnostic to identify patients who may benefit from treatment with the targeted therapies. Additionally, the F1CDx assay can provide tumor mutation profiling to be used in oncology for patients with solid malignant neoplasms.

Genes with full coding exonic regions assessed in the F1CDx assay for the detection of substitutions, insertion-deletions (indels), and copy-number alterations (CNAs) are listed in Table 1. Further details of the FoundationOne CDx assay can be found at www.accessdata.fda.gov/cdrh_docs/pdf17/P170019B.pdf some details of which are set forth below.

FoundationOne CDx (F1CDx) is a single-site assay performed at Foundation Medicine, Inc. The assay includes reagents, software, instruments and procedures for testing DNA extracted from formalin-fixed, paraffin-embedded (FFPE) tumor samples. The assay employs a single DNA extraction method from routine FFPE biopsy or surgical resection specimens, 50-1000 ng of which undergoes whole-genome shotgun library construction and hybridization-based capture of all coding exons from over 300 cancer-related genes, 1 promoter region, 1 non-coding RNA (ncRNA), and select intronic regions from 34 commonly rearranged genes, 21 of which also include the coding exons (refer to FoundationOne CDx for complete list of genes included in F1CDx). In total, the assay therefore detects alterations in up to 324 or more genes. Using the Illumina® HiSeq 4000 platform, hybrid-capture-selected libraries are sequenced to high uniform depth (targeting >500× median coverage with >99% of exons at coverage >100×). Sequence data is processed using a customized analysis pipeline designed to detect all classes of genomic alterations, including base substitutions, indels, copy number alterations (amplifications and homozygous deletions), and selected genomic rearrangements (e.g., gene fusions). Additionally, genomic signatures including microsatellite instability (MSI) and tumor mutational burden (TMB) are reported.

Specimen Collection and Preparation. Formalin-fixed, paraffin-embedded (FFPE) tumor specimens are collected and prepared following standard pathology practices. FFPE specimens may be received either as unstained slides or as an FFPE block. Prior to starting the assay, a Hematoxylin and Eosin (H&E) stained slide is prepared, and then reviewed by a board-certified pathologist to confirm disease ontology and to ensure that adequate tissue (0.6 mm³), tumor content (≥20% tumor) and sufficient nucleated cells are present to proceed with the assay.

Tumor mutational burden (TMB). TMB is measured by counting all synonymous and nonsynonymous variants present at 5% allele frequency or greater, and filtering out potential germline variants according to published databases of known germline polymorphisms including Single Nucleotide Polymorphism database (dbSNP) and Exome Aggregation Consortium (ExAC). Additional germline alterations still present after database querying are assessed for potential germline status and filtered out using a somatic-germline/zygosity (SGZ) algorithm. Furthermore, known and likely driver mutations are filtered out to exclude bias of the data set. The resulting mutation number is then divided by the coding region corresponding to the number of total variants counted, or 793 kb. The resulting number is communicated as mutations per Mb unit (mut/Mb).

TMB-High (TBM-H) corresponds to about 20 or more somatic mutations per megabase (Muts/Mb); TMB-I corresponds to between about 6 and about 19 Muts/Mb; TMB-Low corresponds to about 5 or less Muts/Mb.

### Adjudicated VUS (Variations of Unknown Significance)

A VARsome evaluation algorithm was used to evaluate genetic variations within the Replication Fork genes that were identified using the FoundationOne CDx assay but were not characterized as pathogenic. Some genetic abnormalities identified as VUS were associated with responsiveness to Chk1i therapy.

### Results

An intermediate TMB level (TMB-I) can be a biomarker for SR737 therapy in combination with LDG for a variety of cancers, e.g., anogenital cancers, such as anal cancer. The classification of TMB and impact of TMB may be tumor specific and there is an emerging, yet strong clinical correlation to TMB and immunotherapy activity.

FIG. 27A-27B shows waterfall plots indicating response to treatment with SRA737 in combination with low dose gemcitabine of subjects having particular tumors: anogenital (e.g., anal), rectal, high grade serous ovarian cancer (HGSOC) and cervical. Subjects having at least an intermediate TMB are indicated.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating cancer in a subject, the method comprising:
administering a therapeutically effective amount of a Checkpoint kinase 1 (Chk1) inhibitor to a subject identified as having cancer cells with a genetic abnormality in one or more genes selected from cell cycle regulation genes, replication stress genes, DNA damage response and repair network genes, and oncogenic driver genes.

2. The method of claim 1, wherein the genetic abnormality is an alteration, amplification, overexpression, or underexpression of the one or more genes.

3. The method of claim 1 or 2, wherein the one or more genes is selected from cell cycle regulation genes associated with the G1/S checkpoint and/or the p53 pathway.

4. The method of claim 120, wherein the one or more genes is selected from MDM2, TP53, RB1, CDKN1A/B and CDKN2A/B/C.

5. The method of claim 1 or 2, wherein the one or more genes is selected from replication stress genes implicated in Chk1 pathway sensitivity.

6. The method of claim 5, wherein the one or more genes is selected from Chk1 and ATR.

7. The method of claim 1 or 2, wherein the one or more genes is selected from DNA damage response and repair genes associated with homologous recombination (HR), nonhomologous end joining (NHEJ), Fanconi anemia (FA), or mismatch repair, and a gene encoding chromatin or DNA polymerase.

8. The method of claim 7, wherein the one or more genes is selected from PALB2, ATM, BRCA1/A2, RAD51B, RAD51C, PRKDC, CDK12, FANCA, FANCD2, FANCE, FANCG, FANCI, FANCM, RAD52, RAD50, RAD51C, RAD54L, MLL2, ARID1A, ARID1B, MLH1, MSH2, MSH6, PMS2, POLD1, and POLE.

9. The method of claim 1 or 2, wherein the one or more genes are oncogenic driver genes of the following subclasses: CCNE, MYC and PI3K/AKT.

10. The method of claim 9, wherein the one or more genes are of the CCNE subclass and selected from CCNE1, FBXW7, and PARK2.

11. The method of claim 9, wherein the one or more genes are of the MYC subclass and selected from MYC, MYCN, and MYCL1.

12. The method of claim 9, wherein the one or more genes are of the PI3K/AKT subclass and selected from PIK3CA, PTEN, AKT1, AKT2, and AKT3.

13. The method of claim 7, wherein the one or more genes is selected from DNA damage response and repair genes of the FA/BRCA replication fork subclass.

14. The method of claim 13, wherein the one or more genes of the FA/BRCA replication fork subclass is selected from BRCA1/2, RAD51B, RAD51C, PRKDC, CDK12, FANCA, FANCD2, FANCE, FANCG, FANCI, FANCM, RAD52, RAD50, RAD51C, and RAD54L.

15. The method of any one of claims 1-14, wherein the subject is identified as having cancer lacking a RAS mutation (e.g., a KRAS, HRAS, or NRAS mutation).

16. The method of claim 15, wherein the subject is identified as having cancer with a genetic abnormality in two or more genes selected from oncogenic driver genes of the PI3K/AKT subclass and DNA damage response and repair genes of the FA/BRCA replication fork subclass.

17. The method of claim 16, wherein the two or more genes are selected from AKT, PIK3CA, PTEN, ATR, PRKDC, BRCA1, BRCA2, CDK12, FANCA, FANCD2, FANCE, FANCG, FANCI, FANCM, RAD52, RAD50, RAD51C, and RAD54L.

18. The method of any one of claims 1 to 17, wherein the subject is identified as having at least an intermediate tumor mutational burden (TMB).

19. The method of any one of claims 1 to 18, wherein the cancer is a squamous cell carcinoma.

20. The method of any one of claims 1 to 18, wherein the cancer is selected from advanced/metastatic squamous cell carcinoma of the anus, penis, vagina, and vulva.

21. The method of any one of claims 1 to 18, wherein the cancer is selected from anogenital, rectal, ovarian, and cervical.

22. The method of claim 21, wherein the cancer is anogenital cancer.

23. The method of claim 22, wherein the cancer is anal.

24. The method of claim 21, wherein the cancer is rectal.

25. The method of claim 21, wherein the cancer is cervical.

26. The method of claim 25, wherein the cancer is squamous cervical.

27. The method of claim 21, wherein the cancer is ovarian.

29. The method of claim 27, wherein the ovarian cancer is high-grade serous ovarian cancer (HGSOC).

30. The method of any one of claims 1 to 29, wherein the cancer is positive for human papillomavirus (HPV).

31. The method of any one of claims 1 to 30, wherein the Chk1 inhibitor is SRA737.

32. The method of claim 31, wherein the SRA737 compound is administered orally.

33. The method of any of claims 31 to 32, wherein the SRA737 compound is administered daily.

34. The method of any of claims 31 to 32, wherein the SRA737 compound is administered every other week.

35. The method of any one of claims 31 to 32 wherein the SRA737 compound is administered intermittently.

36. The method of claim 35, wherein the SRA737 compound is administered with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations.

37. The method of any one of claims 31 to 36, wherein the SRA737 compound is administered over one or more 28 day cycles.

38. The method of claim 37, wherein the SRA737 compound is administered on one or more days of the one or more 28 day cycles (e.g., 2, 3, 4, 5, 6 or more 28 day cycles).

39. The method of claim 38, wherein the SRA737 compound is administered every other week of the one or more 28 day cycles.

40. The method of any one of claims 37-39, further comprising administering an initial dose of the SRA737 compound prior to the first of the one or more 28 day cycles.

41. The method of claim 40, wherein the initial dose is administered 4 days, 5 days, 6 days, or 7 days prior to the first cycle of the one or more 28 day cycles.

42. The method of any one of claims 31 to 36, wherein the SRA737 compound is administered following a dosing schedule selected from: 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; and dosing on days 2 and 3 of a weekly cycle.

43. The method of any one of claims 31 to 42, wherein the effective amount is administered in a single dose once a day.

44. The method of any one of claims 31 to 42, wherein half of the effective amount is administered twice a day.

45. The method of any one of claims 31 to 44, wherein the effective amount is 1000 mg/day or less (e.g., 900 mg/day or less, 800 mg/day or less, 700 mg/day or less, 600 mg/day or less, 500 mg/day or less, 400 mg/day or less, 300 mg/day or less, or even less).

46. The method of any one of claims 31 to 45, wherein the effective amount is between 300 mg/day and 1000 mg/day (e.g., between 300 mg/day and 900 mg/day, or between 300 mg/day and 800 mg/day; or between 500 mg/day and 1000 mg/day, or between 500 mg/day and 800 mg/day).

47. The method of any one of claims 31 to 46, wherein the effective amount is selected from: 300 mg/day, 400 mg/day, 500 mg/day, 600 mg/day, 700 mg/day, 800 mg/day, 900 mg/day, and 1000 mg/day.

48. The method of any one of claims 1 to 47, further comprising administering gemcitabine to the subject in combination with the Chk1 inhibitor.

49. The method of claim 48, wherein an effective amount of the gemcitabine administered is sub-therapeutic.

50. The method of claim 48 or 49, wherein the gemcitabine is administered daily.

51. The method of any one of claims 48 to 50, wherein the gemcitabine is administered on day 1 and the Chk1 inhibitor is administered on days 2 and 3 of a weekly schedule.

52. The method of any one of claims 48 to 51, wherein the gemcitabine and the Chk1 inhibitor are administered over one or more 28 day cycles.

53. The method of claim 52, wherein the gemcitabine is administered on days 1, 8, and 15 of the one or more 28 day cycles, and the Chk1 inhibitor is administered on days 2, 3, 9, 10, 16, and 17 of the one or more 28 day cycles.

54. The method of any one of claims 48 to 53, wherein an effective amount of the gemcitabine administered is selected from: 50 mg/m²/day, 100 mg/m²/day, 150 mg/m²/day, 200 mg/m²/day, 250 mg/m²/day, and 300 mg/m²/day.

55. The method of any one of claims 48 to 53, wherein an effective amount of the gemcitabine administered is 600 mg/m²/day or less (e.g., between 50 and 600 mg/m²/day, or between 50 and 300 mg/m²/day).

56. The method of any one of claims 54 to 55, wherein the therapeutically effective amount of the SRA737 compound is 150 mg/day.

57. The method of any one of claims 54 to 55, wherein the therapeutically effective amount of the SRA737 compound is 300 mg/day.

58. The method of any one of claims 54 to 55, wherein the therapeutically effective amount of the SRA737 compound is 500 mg/day.

59. The method of any one of claims 54 to 55, wherein the effective amount of the SRA737 compound is 600 mg/day.

60. The method of any one of claims 54 to 55, wherein the effective amount of the SRA737 compound is 700 mg/day.

61. The method of any one of claims 54 to 55, wherein the effective amount of the SRA737 compound is 800 mg/day.

62. The method of any one of claims 54 to 55, wherein the effective amount of the SRA737 compound is 900 mg/day.

63. The method of any one of claims 54 to 55, wherein the effective amount of the SRA737 compound is 1000 mg/day.

64. The method of any one of claims 1 to 63, further comprising administering an immune checkpoint inhibitor to the subject.

65. The method of claim 64, wherein the immune checkpoint inhibitor is a monoclonal antibody.

66. The method of claim 65, wherein the monoclonal antibody is an anti-PD-1 or anti-PD-L1 antibody.

67. The method of claim 65, wherein the monoclonal antibody is an anti-CTLA-4 antibody.

68. A method for selecting a subject for Chk1 inhibitor therapy, the method comprising:
determining whether there is a mutation in RAS in cancer cells from a subject having cancer;
determining whether there is a genetic abnormality in one or more genes selected from cell cycle regulation genes, replication stress genes, DNA damage response and repair network genes, and oncogenic driver genes in cancer cells from the subject; and
categorizing the subject as one who will benefit from Chk1 inhibitor therapy when the cancer cells from the subject lack a mutation in RAS and comprise a genetic abnormality in the one or more genes.

69. The method of claim 68, wherein the one or more genes is two or more genes.

70. The method of claim 68 or 69, wherein the genetic abnormality is an alteration, amplification, overexpression, or underexpression of the one or more genes.

71. A method for selecting a subject for Chk1 inhibitor therapy, the method comprising:
determining whether there is a mutation in RAS in cancer cells from a subject having cancer;
determining whether there is a genetic abnormality in one or more genes selected from cell cycle regulation genes, replication stress genes, DNA damage response and repair network genes, and oncogenic driver genes in cancer cells from the subject; and
categorizing the subject as one who will benefit from Chk1 inhibitor therapy when the cancer cells from the subject lack a mutation in RAS and comprise a genetic abnormality in the one or more genes.

72. The method of claim 71, wherein the one or more genes is two or more genes.

73. The method of claim 70 or 71, wherein the genetic abnormality is an alteration, amplification, overexpression, or underexpression of the one or more genes.

74. A method of treating cancer in a subject, the method comprising:
administering a therapeutically effective amount of a Checkpoint kinase 1 (Chk1) inhibitor to a subject identified as having (i) cancer and (ii) at least an intermediate tumor mutational burden (TMB).

75. The method of claim 74, further comprising:
determining if a subject having cancer has a tumor with at least an intermediate tumor mutational burden (TMB); and then
administering to a subject determined to have a tumor with at least an intermediate TMB a therapeutically effective amount of a Chk1 inhibitor.

76. The method of any one of claims 74 to 75, wherein an intermediate TMB is determined by comparing a TMB value determined from a sample from the subject to a reference TMB value indicative of responsiveness to Chk1 inhibitor therapy.

77. The method of claim 76, wherein the TMB value represents a number of somatic alterations counted over a defined number of nucleic acid base pairs or genes.

78. The method of claim 77, wherein the defined number of nucleic acid base pairs are coding nucleic acid base pairs.

79. The method of claim 77 or 78, wherein the number of somatic alterations is determined by a method comprising sequencing a predetermined set of genes set forth in Table 1 (e.g., sequencing the coding regions of the predetermined set of genes set forth in Table 1, such as a predetermined set of genes comprising 50 or more, 100 or more, 150 or more, 200 or more, 250 or more, 300 or more, or all of the genes set forth in Table 1).

80. The method of claim 79, wherein a low TMB is indicative of the level of somatic alterations in a predetermined set of genes in a sample derived from a tumor from a non-responder to the therapy.

81. The method of any one of claims 76-80, wherein the reference TMB value is about 5 or more (e.g., about 5.5 or more, about 6 or more, about 6.5 or more, about 7 or more, about 8 or more, about 9 or more, about 10 or more, about 15 or more, about 20 or more, about 25 or more, about 30 or more, about 35 or more, about 40 or more, about 45 or more, or about 50 or more) somatic alterations per megabase (Mb) in the coding regions of the predetermined set of genes.

82. The method of any one of claims 76-81, wherein the reference TMB value is about 6 to about 10 (e.g., about 6, about 7, about 8, about 9 or about 10) somatic alterations per megabase (Mb) in a predetermined set of genes comprising 50 or more, 100 or more, 150 or more, 200 or more, 250 or more, 300 or more, or all of the genes set forth in Table 1.

83. The method of claim 82, wherein the reference TMB value is about 6 somatic alterations per megabase (Mb) of coding sequence.

84. The method of any one of claims 82 to 83, wherein the intermediate TMB is about 6 to about 19 somatic alterations per megabase (Mb) of coding sequence.

85. The method of any one of claims 82 to 84, wherein a high TMB is about 20 or more somatic alterations per megabase (Mb) of coding sequence.

86. The method of any one of claims 76 to 85, wherein the sample is selected from tissue sample, whole blood sample, plasma sample, and serum sample.

87. The method of any one of claims 76 to 86, wherein the sample comprises tissue obtained from the subject.

88. The method of any one of claims 76 to 87, wherein the sample comprises tumor cells.

89. The method of claim 88, wherein the sample obtained from the subject contains at least 20% tumor cells.

90. The method of any one of claims 74 to 89, wherein the Chk1 inhibitor is SRA737.

91. The method of claim 90, wherein the SRA737 compound is administered orally.

92. The method of any of claims 90 to 91, wherein the SRA737 compound is administered daily.

93. The method of any of claims 90 to 92, wherein the SRA737 compound is administered every other week.

94. The method of any one of claims 90 to 92, wherein the SRA737 compound is administered intermittently.

95. The method of claim 94, wherein the SRA737 compound is administered with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations.

96. The method of any one of claims 90 to 95, wherein the SRA737 compound is administered over one or more 28 day cycles.

97. The method of claim 96, wherein the SRA737 compound is administered on one or more days of the one or more 28 day cycles (e.g., 2, 3, 4, 5, 6 or more 28 day cycles).

98. The method of claim 97, wherein the SRA737 compound is administered every other week of the one or more 28 day cycles.

99. The method of any one of claims 96-98, further comprising administering an initial dose of the SRA737 compound prior to the first of the one or more 28 day cycles.

100. The method of claim 99, wherein the initial dose is administered 4 days, 5 days, 6 days, or 7 days prior to the first cycle of the one or more 28 day cycles.

101. The method of any one of claims 96 to 100, wherein the SRA737 compound is administered following a dosing schedule selected from: 5 days of dosing followed by 2 days of non-dosing each week; 1 week of daily dosing followed by 1, 2, or 3 weeks of non-dosing; 2 or 3 weeks of daily dosing followed by 1, or 2 weeks of non-dosing; and dosing on days 2 and 3 of a weekly cycle.

102. The method of any one of claims 90 to 101, wherein the effective amount is administered in a single dose once a day.

103. The method of any one of claims 90 to 101, wherein half of the effective amount is administered twice a day.

104. The method of any one of claims 90 to 103, wherein the effective amount is 1000 mg/day or less (e.g., 900 mg/day or less, 800 mg/day or less, 700 mg/day or less, 600 mg/day or less, 500 mg/day or less, 400 mg/day or less, 300 mg/day or less, or even less).

105. The method of any one of claims 90 to 104, wherein the effective amount is between 300 mg/day and 1000 mg/day (e.g., between 300 mg/day and 900 mg/day, or between 300 mg/day and 800 mg/day; or between 500 mg/day and 1000 mg/day, or between 500 mg/day and 800 mg/day).

106. The method of any one of claims 90 to 105, wherein the effective amount is selected from: 300 mg/day, 400 mg/day, 500 mg/day, 600 mg/day, 700 mg/day, 800 mg/day, 900 mg/day, and 1000 mg/day.

107. The method of any one of claims 74-106, further comprising administering gemcitabine to the subject in combination with the Chk1 inhibitor.

108. The method of claim 107, wherein an effective amount of the gemcitabine administered is sub-therapeutic.

109. The method of claim 107 or 108, wherein the gemcitabine is administered daily.

110. The method of any one of claims 107 to 109, wherein the gemcitabine is administered on day 1 and the Chk1 inhibitor is administered on days 2 and 3 of a weekly schedule.

111. The method of any one of claims 107 to 110, wherein the gemcitabine and the Chk1 inhibitor are administered over one or more 28 day cycles.

112. The method of claim 111, wherein the gemcitabine is administered on days 1, 8, and 15 of the one or more 28 day cycles, and the Chk1 inhibitor is administered on days 2, 3, 9, 10, 16, and 17 of the one or more 28 day cycles.

113. The method of any one of claims 107 to 112, wherein an effective amount of the gemcitabine administered is selected from: 50 mg/m²/day, 100 mg/m²/day, 150 mg/m²/day, 200 mg/m²/day, 250 mg/m²/day, and 300 mg/m²/day.

114. The method of any one of claims 107 to 112, wherein an effective amount of the gemcitabine administered is 600 mg/m²/day or less (e.g., between 50 and 600 mg/m²/day, or between 50 and 300 mg/m²/day).

115. The method of any one of claims 113 to 114, wherein the therapeutically effective amount of the SRA737 compound is 80 or 150 mg/day.

116. The method of any one of claims 113 to 114, wherein the therapeutically effective amount of the SRA737 compound is 300 mg/day.

117. The method of any one of claims 113 to 114, wherein the therapeutically effective amount of the SRA737 compound is 500 mg/day.

118. The method of any one of claims 113 to 114, wherein the effective amount of the SRA737 compound is 600 mg/day.

119. The method of any one of claims 113 to 114, wherein the effective amount of the SRA737 compound is 700 mg/day.

120. The method of any one of claims 113 to 114, wherein the effective amount of the SRA737 compound is 800 mg/day.

121. The method of any one of claims 113 to 114, wherein the effective amount of the SRA737 compound is 900 mg/day.

122. The method of any one of claims 113 to 114, wherein the effective amount of the SRA737 compound is 1000 mg/day.

123. The method of any one of claims 71-122, further comprising administering an immune checkpoint inhibitor to the subject.

124. The method of claim 123, wherein the immune checkpoint inhibitor is a monoclonal antibody.

125. The method of claim 124, wherein the monoclonal antibody is an anti-PD-1 or anti-PD-L1 antibody.

126. The method of claim 124, wherein the monoclonal antibody is an anti-CTLA-4 antibody.

127. The method of any one of claims 71 to 126, wherein the cancer is a squamous cell carcinoma.

128. The method of any one of claims 71 to 126, wherein the cancer is selected from advanced/metastatic squamous cell carcinoma of the anus, penis, vagina, and vulva.

129. The method of any one of claims 71 to 126, wherein the cancer is selected from anogenital, rectal, ovarian, and cervical.

130. The method of claim 129, wherein the cancer is anogenital cancer.

131. The method of claim 130, wherein the cancer is anal.

132. The method of claim 129, wherein the cancer is cervical.

133. The method of claim 132, wherein the cancer is squamous cervical.

134. The method of claim 129, wherein the cancer is ovarian.

135. The method of claim 134, wherein the ovarian cancer is high-grade serous ovarian cancer (HGSOC).

136. The method of any one of claims 71 to 135, wherein the cancer is positive for human papillomavirus (HPV).

137. The method of any one of claims 71 to 136, wherein a tumor associated with the cancer is identified as having a genetic abnormality (e.g., an alteration, amplification, overexpression, or underexpression) in one or more genes selected from cell cycle regulation genes, replication stress genes, DNA damage response and repair network genes, and oncogenic driver genes.

138. The method of claim 137, wherein the one or more genes is selected from DNA damage response and repair genes associated with homologous recombination (HR), nonhomologous end joining (NHEJ), Fanconi anemia (FA), or mismatch repair, and a gene encoding chromatin or DNA polymerase.

139. The method of claim 138, wherein the one or more genes is selected from PALB2, ATM, BRCA1/A2, RAD51B, RAD51C, PRKDC, CDK12, FANCA, FANCD2, FANCE, FANCG, FANCI, FANCM, RAD52, RAD50, RAD51C, RAD54L, MLL2, ARID1A, ARID1B, MLH1, MSH2, MSH6, PMS2, POLD1, and POLE.

140. The method of claim 137, wherein the one or more genes are of the PI3K/AKT subclass and selected from PIK3CA, PTEN, AKT1, AKT2, and AKT3.

141. The method of claim 137, wherein the one or more genes is selected from DNA damage response and repair genes of the FA/BRCA replication fork subclass.

142. The method of claim 141, wherein the one or more genes of the FA/BRCA replication fork subclass is selected from BRCA1/2, RAD51B, RAD51C, PRKDC, CDK12, FANCA, FANCD2, FANCE, FANCG, FANCI, FANCM, RAD52, RAD50, RAD51C, and RAD54L.

143. The method of any one of claims 137-142, wherein the subject is identified as having cancer cells with wild type RAS (e.g., wt KRAS, wt HRAS, wt NRAS).

144. The method of claim 143, wherein the subject is identified as having cancer cells with a genetic abnormality in two or more genes selected from oncogenic driver genes of the PI3K/AKT subclass and DNA damage response and repair genes of the FANC/BRCA replication fork subclass.

145. The method of claim 144, wherein the two or more genes are selected from AKT, PIK3CA, PTEN, ATR, PRKDC, BRCA1, BRCA2, CDK12, FANCA, FANCD2, FANCE, FANCG, FANCI, FANCM, RAD52, RAD50, RAD51C, and RAD54L.

146. A method for selecting a subject for Chk1 inhibitor therapy, the method comprising:
determining a tumor mutational burden (TMB) value from a sample from a subject having cancer;
comparing the determined TMB value from the sample to a reference TMB value indicative of responsiveness to Chk1 inhibitor therapy; and
categorizing the subject as one who will benefit from Chk1 inhibitor therapy when the determined TMB value is at or above the reference TMB value.

147. The method of claim 146, wherein the reference TMB value is less than or equal to a median TMB value determined from a plurality of tumor cell samples obtained from a plurality of subjects having cancer.

148. The method of any one of claims 146 to 147, wherein the reference TMB value is a pre-assigned TMB value.

149. The method of any one of claims 146 to 148, wherein the reference TMB value represents a number of somatic alterations counted over a defined number of nucleic acid base pairs.

150. The method of any one of claims 146 to 149, wherein the reference TMB value is determined from a plurality of samples obtained from a plurality of subjects having a cancer selected from anal, rectal, ovarian, and cervical.

151. The method of any one of claims 146 to 150, wherein a TMB value represents a number of somatic alterations counted over a defined number of nucleic acid base pairs or genes.

152. The method of claim 151, wherein the defined number of nucleic acid base pairs are coding nucleic acid base pairs.

153. The method of claim 151 or 152, wherein the defined number of coding nucleic acid base pairs is one (1) million (1 megabase (Mb)) or greater.

154. The method of claim 151 or 152, wherein the defined number of coding nucleic acid base pairs is a whole exome.

155. The method of any one of claims 151 to 154, wherein the somatic alterations are known somatic alterations in COSMIC.

156. The method of any one of claims 151 to 155 wherein truncations in tumor suppressor genes are not counted as somatic alterations.

157. The method of any one of claims 151 to 156, wherein alterations predicted to be germline by the somatic-germline-zygosity algorithm are not counted as somatic alterations.

158. The method of any one of claims 151 to 157, wherein the number of somatic alterations is determined by a method comprising sequencing a predetermined set of genes set forth in Table 1 (e.g., sequencing the coding regions of the predetermined set of genes set forth in Table 1, such as a predetermined set of genes comprising 50 or more, 100 or more, 150 or more, 200 or more, 250 or more, 300 or more, or all of the genes set forth in Table 1).

159. The method of any one of claims 151 to 158, wherein a low TMB is indicative of the level of somatic alterations in a predetermined set of genes set forth in Table 1 in a sample derived from a tumor from a non-responder to the therapy.

160. The method of any one of claims 151 to 159, wherein the reference TMB value is about 5 or more (e.g., about 5.5 or more, about 6 or more, about 6.5 or more, about 7 or more, about 8 or more, about 9 or more, about 10 or more, about 15 or more, about 20 or more, about 25 or more, about 30 or more, about 35 or more, about 40 or more, about 45 or more, or about 50 or more) somatic alterations per megabase in coding regions of a predetermined set of genes.

161. The method of any one of claims 151 to 160, wherein the reference TMB value is about 6 to about 10 (e.g., about 6, about 7, about 8, about 9 or about 10) somatic alterations per megabase (Mb) in a predetermined set of genes comprising 50 or more, 100 or more, 150 or more, 200 or more, 250 or more, 300 or more, or all of the genes set forth in Table 1.

162. The method of claim 161, wherein the reference TMB value is about 6 somatic alterations per megabase (Mb).

163. The method of claim 160, wherein an intermediate TMB is about 6 to about 19 somatic alterations per megabase (Mb).

164. The method of any one of claims 160 to 163, wherein a high TMB is about 20 or more somatic alterations per megabase (Mb).

165. The method of any one of claims 151 to 164, wherein the sample is selected from tissue sample, whole blood sample, plasma sample, and serum sample.

166. The method of any one of claims 151 to 165, wherein the sample comprises tissue obtained from the subject.

167. The method of any one of claims 151 to 66, wherein the sample comprises tumor cells.

168. The method of claim 167, wherein the sample obtained from the subject contains at least 20% tumor cells.

169. The method of any one of claims 165 to 168, wherein the sample is an archival sample (e.g., formalin-fixed), a fresh sample, or a frozen sample.

170. The method of any one of claims 151 to 169, further comprising administering a therapeutically effective amount of a Chk1 inhibitor to the subject determined to have at least an intermediate TMB.

171. The method of claim 170, wherein the Chk1 inhibitor is SRA737.

172. The method of any one of claims 170 to 171, further comprising administering gemcitabine to the subject in combination with the Chk1 inhibitor.

173. The method of any one of claims 170-172, further comprising administering an immune checkpoint inhibitor to the subject.

174. The method of claim 173, wherein the immune checkpoint inhibitor is a monoclonal antibody.

175. The method of claim 174, wherein the monoclonal antibody is an anti-PD-1 antibody, an anti-PD-L1 antibody, or an anti-CTLA-4 antibody.

176. The method of any one of claims 1-175, wherein the subject is human.
